(19) United States
(12) Patent Application Publication
(10) Pub. No.: US 2026/0216120 A1
(43) Pub. Date: Jul. 30, 2026

(54) PLATINUM COMPLEXES, RELATED COMPOSITIONS, AND USES THEREOF IN CYANIDE COUNTER-MEASURES

(71) Applicants: Matthew Brenner, Irvine, CA (US); Purdue Research Foundation, West Lafayette, IN (US); The Regents of University of California, Oakland, CA (US); The Brigham and Women's Hospital, Inc., Boston, MA (US); University of Utah Research Foundation, Salt Lake City, UT (US); Beth Israel Deaconess Medical Center, Inc., Boston, MA (US)

(72) Inventors: Vincent Jo Davisson, Lafayette, IN (US); Gregory Thomas Knipp, West Lafayette, IN (US); Naoaki Fujii, Germantown, TN (US); Matthew Mark Behymer, West Lafayette, IN (US); Huaping Mo, Carmel, IN (US); Gerard Boss, La Jolla, CA (US); Matthew Brenner, Irvine, CA (US); Calum A. Macrae, Wellesley Hills, MA (US); Anjali Nath, Boston, MA (US); Robert E. Gerszten, Brookline, MA (US); Randall Peterson, Salt Lake City, UT (US)

(21) Appl. No.: 19/099,746

(22) PCT Filed: Jul. 28, 2023
(86) PCT No.: PCT/US2023/029041

§ 371 Date: Jan. 29, 2025

Related U.S. Application Data
(60) Provisional application No. 63/393,647, filed on Jul. 29, 2022.

(51) Int. Cl. A61K 31/282; A61K 31/19; A61M 5/19; A61P 39/02; C07F 15/00

(52) U.S. Cl. CPC A61K 31/282; A61K 31/19; A61M 5/19; A61P 39/02; C07F 15/0093

(57) ABSTRACT

Platinum-thioester complexes, compositions comprising such platinum-thioester complexes, methods of treating cyanide poisoning and/or cyanide exposure with the same and related combinations therapies and kits therefore.

### PRIORITY

This application is related to and claims the priority benefit of U.S. Provisional Patent Application No. 63/393,647 filed Jul. 29, 2022. The content of the aforementioned application is hereby incorporated by reference in its entireties into this disclosure.

### GOVERNMENT FUNDING

This invention was made with government support under NS 112107 (CM and RP) awarded by the National Institutes of Health (NIH) and under P200A150136 awarded by the U.S. Department of Education Graduate Assistance in Areas of Nation Need. The government has certain rights in the invention.

### TECHNICAL FIELD

The present disclosure generally relates to platinum-containing complexes and methods for treating (e.g., providing an antidote to) cyanide poisoning by administering the platinum-complex to a subject exposed to cyanide (e.g., cyanide gas).

### BACKGROUND

Cyanide anions (CN⁻) are highly toxic due to their ability to disrupt electron transport in a cell, which can lead to the cell's inability to aerobically produce adenosine triphosphate (ATP) for energy. Cyanide is a persistent threat for accidental and malicious misuse due to its ease of generation as a toxic gas and widespread access across multiple industries. Exposure to cyanide can be fatal even when small amounts are inhaled, ingested, or directly contacted, and can kill organisms as diverse as insects, fish, and humans within seconds to hours.

A leading cause of smoke inhalation-related deaths is suspected to be a result of cyanide poisoning. Cyanide can be released into the smoke when carbon and nitrogen-rich materials in everyday household items are burned. Anseeuw et al., Cyanide poisoning by fire smoke inhalation: a European expert consensus, European J Emergency Med 20: 2-9 (2013). Household fires resulting in the combustion of plastics and plant-based materials, including wood, can also be sources of cyanide. For example, cyanide emitted from the burning of soundproofing plastics has been associated with high morbidity and mortality events in nightclubs, claiming the lives of over 4200 individuals in a single event in Brazil. Cyanide and cyanide derivatives are often used in many industries, such as metal polishing, mining, photographic development, and the chemical production of products like pesticides. Bhattacharya & Flora, Cyanide toxicity and its treatment, Handbook of Toxicology of Chem Warfare Agents, 301-314 (2015); Use in Mining, Int'l Cyanide Management Code (ICMI) for the Manufacture, Transport and Use of Cyanide in the Production of Gold. In total, around 1.1 million metric tons of cyanide are produced annually for industrial applications. Use in Mining, supra. Cyanide production is also easily achieved, as it uses readily available ingredients such as ammonia and methane. Grabow et al., Descriptor-based analysis applied to HCN synthesis from NH₃ and CH₄, Angewandte Chemi 50(20): 4601-4605 (2011). In the presence of acid, hydrogen cyanide is volatile and can fill the immediate air with deadly gas.

Based on its widespread availability and easy gas conversion, cyanide is a threat for malicious misuse to induce high mortality and morbidity (e.g., such as a chemical weapon). Examples of such attacks include a subway attack in Tokyo, where terrorists placed cyanide salt and acid into trash cans in an attempt to fill the station with lethal gas, and the incident that occurred in 1978 at Jonestown in Guyana. Kristof, How Tokyo barely escaped even deadlier subway attack, New York Times, May 18, 1995; Conroy, An apocalyptic cult, 900 dead: remembering the Jonestown massacre, 40 years on, The Guardian, Nov. 17, 2018.

Cyanide gas is difficult to detect and has a rapid toxic onset; within minutes, a dose as low as 2 mg/kg can often be lethal. Within the time required for emergency responders to arrive and administer treatment, toxic effects of cyanide can already be advanced. In the case of lethal acute cyanide exposure, mortality and morbidity can occur within the first 30-60 minutes, thus representing an unmet medical need for rapid-acting countermeasures.

Cyanide is a systemic poison that inhibits cellular respiration by reversible inhibition of cytochrome C oxidase in the mitochondria. Levels NRC (US) S on AEG, Hydrogen cyanide: acute exposure guideline levels, National Academies Press (2002); Leavesley et al., Interaction of cyanide and nitric oxide with cytochrome c oxidase: implications for acute cyanide toxicity, Toxicological Sciences 101(1): 101-111 (2008). This inhibition can manifest clinically as cardiac arrhythmias, seizures, behavioral disturbance or even as an imbalance of normal oxy-/deoxygenated hemoglobin homeostasis.

The fast action of cyanide on physiological functions requires rapid treatment options to reduce morbidity and mortality. Approximately 50% of mice exposed to constant atmospheric cyanide concentrations as low as 177 parts per million (ppm) died within 30 minutes of exposure. Alarie, Toxicity of Fire Smoke, Critical Reviews in Toxicology 32: 259-289 (2022).

FDA-approved treatments for cyanide exposure include hydroxocobalamin, sodium nitrite, and sodium thiosulfate. Dicobalt edetate is also used in Europe only in cases of severe cyanide intoxication due to the compound's adverse effects. However, administration of current cyanide countermeasures is by intravenous (IV) infusion or inhalation. For example, 5 grams of hydroxocobalamin is typically required to be administered by IV infusion over 15 minutes, occasionally with the need for repeat dosing. CYANOKIT® hydroxocobalamin for injection) for intravenous infusion, Food & Drug Admin, Reference ID: 4369589. In some cases, higher doses over a longer time period can be required, which can significantly increase the risk of co-morbidities including drug-induced renal impairment. Further, evidence strongly supports that a prompt delivery of cyanide scavengers following exposure can significantly improve survival rates. Thompson et al., Modest and variable efficacy of pre-exposure hydroxocobalamin and dicobalt edetate in a porcine model of acute cyanide salt poisoning, Clinical Toxicology (Philia) 58: 190-200 (2019). However, IV infusion of current scavengers requires trained medical staff and at least several minutes to deliver an entire dose. Meillier & Heller, Acute cyanide poisoning: hydroxocobalamin and sodium thiosulfate treatment with two outcomes following one exposure event, Case Reports in Medicine (2015). Accordingly, existing treatments can take a substantial amount of time to achieve complete administration and pharmacological action, making them less than optimal due to the rapid toxic onset observed with cyanide exposure. In the scenario of a potential mass casualty cyanide exposure, establishing large numbers of IV infusion lines by first responders is not feasible.

A further complication is that the conventional compounds used to treat cyanide exposure (e.g., hydroxocobalamin) have relatively low aqueous solubility (27.3 ng/mL), which can be a confounder with the often-required multiple dosing. Moreover, due to the numerous toxic side effects of conventional treatments, there is ambiguity regarding the best countermeasure options in emergencies. Further compounding matters, conventional active scavenger agents are stoichiometric reactants with cyanide, which increases the dosage requirement of the active pharmaceutical ingredient and can impose additional limitations on routes of administration. The mechanism of action for hydroxocobalamin involves the direct binding of cyanide in a 1:1 stoichiometric ratio at the metal center. Hamel, Review of acute cyanide poisoning with a treatment update, Critical Care Nurse 31: 72-82 (2011).

The discovery and development of rapid-acting cyanide countermeasures that can be delivered via single bolus intramuscular (IM) administration with increased solubility and reduced side effects remains a critical area for investigation.

### SUMMARY

Platinum (Pt)-based compounds comprising bidentate ligands are provided. In certain embodiments, the compound has a structure of Formula (I):

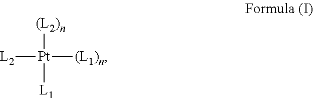

or a pharmaceutically acceptable salt, N-oxide, solvate, tautomer, or stereoisomer thereof, wherein:
Pt is platinum(II);
each L₁ and L₂ is a ligand, wherein each L₁ forms a bidentate ligand and each L₂ forms a bidentate ligand, each bidentate ligand comprising an N, an S, or both an N and an S coordinated to the platinum(II), wherein at least one of the ligands is a leaving group and at least two of the ligands that are directly bonded to the platinum(II) each independently comprise a thioether comprising an alkyl, a carboxyamide, an amine, an amino sulfide, a carboxylate, a carboxyester, a carbonyl, or a combination of any of the foregoing; and
each n is independently −1-5.

In certain embodiments, the compound has a structure of Formula (II):

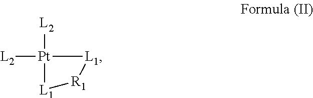

or isa pharmaceutically acceptable salt, N-oxide, solvate, tautomer, or stereoisomer thereof, wherein:
Pt is platinum(II);
each L₁ and L₂ is a ligand, wherein each L₁ forms a bidentate ligand and each L₂ forms a bidentate ligand, each a bidentate ligand comprising an N, an S, or both an N and an S coordinated to the platinum(II), wherein at least one of the ligands is a leaving group and at least two of the ligands each independently comprise a thioether comprising an alkyl, a carboxyamide, a carboxyester, an amine, an amino sulfide, a carboxylate, a carbonyl, or a combination of any of the foregoing;
R₁ is selected from a group consisting of C₁₋₃ alkyl, C₁₋₃ haloalkyl, C₂₋₄ alkenyl, C₂₋₄ alkynyl, and C₆₋₁₀ aryl, or absent; and
each n is independently −1-5.

In certain embodiments, the compound has a structure of Formula (III):

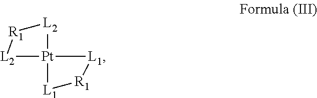

or a pharmaceutically acceptable salt, N-oxide, solvate, tautomer, or stereoisomer thereof, wherein:
Pt is platinum(II);
each L₁ and L₂ is a ligand, wherein each L₁ forms a bidentate ligand and each L₂ forms a bidentate ligand, each a bidentate ligand comprising an N, an S, or both an N and an S coordinated to the platinum(II), wherein at least one of the ligands is a leaving group and at least two of the ligands that are directly bonded to the platinum(II) comprise a thioether comprising an alkyl, a carboxyamide, an amine, an amino sulfide, a carboxylate, a carbonyl, or a combination of any of the foregoing;
each R₁ is selected from a group consisting of C₁₋₃ alkyl, C₁₋₃ haloalkyl, C₂₋₄ alkenyl, C₂₋₄ alkynyl, and C₆₋₁₀ aryl, or absent; and
each n is independently −1-5.

Each L₁ can be a leaving group. Each L₂ can be a leaving group. In certain embodiments, each L₁ and L₂ is a leaving group.

In certain embodiment, each R₁ of Formula (II) or (III) is absent. R₁ can be a C₁₋₃ alkyl.

The compound can comprise a cis configuration. The compound can comprise a trans configuration. A first ligand (e.g., of a bidentate ligand) can comprise a sulfide and a second ligand (e.g., of a bidentate ligand) comprises an amide.

At least one thioester ligand of the compound can comprise an amino sulfide. The thioester ligand(s) can each comprise an amino sulfide.

In certain embodiments, the bidentate ligands can independently comprise 5- or 6-membered bidentate ligands.

The compound can have the following structure:

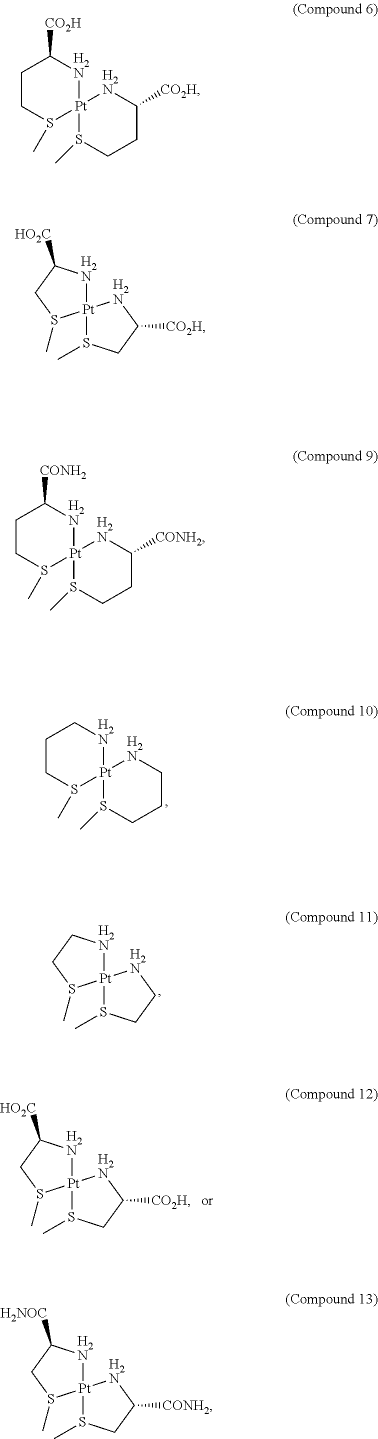

or can be a pharmaceutically acceptable salt, N-oxide, solvate, tautomer, or stereoisomer of any of the foregoing structures.

The compound can comprise the following structure or be a pharmaceutically acceptable salt, N-oxide, solvate, tautomer, or stereoisomer thereof:

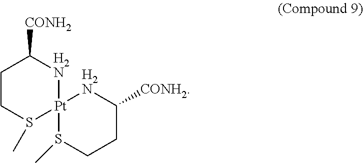

The compound can comprise the following structure or be a pharmaceutically acceptable salt, N-oxide, solvate, tautomer, or stereoisomer thereof:

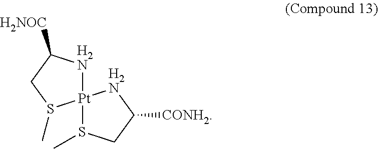

In certain embodiments, at least one of the ligands comprises a methyl thioether group. In certain embodiments, at least one of thioether ligands comprises methionine or S-methylcysteine optionally comprising one or more amidated carboxylates. The bidentate ligands can independently comprise 5- or 6-membered bidentate ligands, and at least one of such bidentate ligands can comprise a carboxylate or carboxamide substituent.

In certain embodiments, at least one of the ligands comprises HCP-AKN, cisplatin-AKN, (SallylCys)₂Pt, (SMePenicillamine)₂P, (cilastatin)₂Pt, or (bridged-Met₂)Pt, MetPt(taurine)₂.

Pharmaceutical compositions are also provided. A pharmaceutical composition hereof can comprise any of the compounds hereof, or a pharmaceutically acceptable salt, N-oxide, solvate, tautomer, or stereoisomer thereof, and a pharmaceutically acceptable carrier and/or diluent. The pharmaceutical composition can further comprise a pharmaceutically acceptable excipient. The composition can be suitable for intramuscular injection.

Uses of a pharmaceutical salt, N-oxide, solvate, tautomer, or stereoisomer of a compound hereof, a pharmaceutical salt, N-oxide, solvate, tautomer, or stereoisomer thereof, or a pharmaceutical composition hereof in the manufacturer of a medicament for the treatment of a disease or condition in a subject are also provided. In certain embodiments, the disease or condition is cyanide poisoning or cyanide exposure. The medicament can be formulated for intramuscular administration. The medicament can be formulated in a single-bolus dosage. The medicament can be formulated at about or above a pH of 5 (such as at about 5, 5, or >5). The medicament can be stored at about or below a pH of 5 (such as at about 5, 5, or >5).

Methods of treating cyanide poisoning or cyanide exposure in a subject are also provided. In certain embodiments, a method of treating cyanide poisoning or cyanide exposure in a subject comprises administering to the subject a first therapy comprising a therapeutically effective amount of: a compound hereof, a pharmaceutically acceptable salt, N-oxide, solvate, tautomer, or stereoisomer of a compound hereof, or a pharmaceutical composition hereof.

In certain embodiments, administering comprises intramuscular injection. In certain embodiments, administering the therapeutically effective amount of the first therapy comprises administering a single dose.

The method can further comprise administering to the subject a second therapy, the second therapy comprising administering to the subject: a therapeutically effective amount of one or more of glyoxylate, hydroxocobalamin, methemoglobin, riboflavin, methotrexate, 4-dimethylaminophenol (4-DMAP), dicobalt edetate, glucose, activated charcoal, and cobalt disodium ethylenediaminetetraacetic acid (EDTA); an isotonic fluid intravenously; and/or oxygen therapy. The first and second therapies can be administered sequentially. The first and second therapies can be administered concurrently. The second therapy can be glyoxylate and the therapeutically effective amount of the first therapy can be about 3.0-5.5 mg/kg (by weight of the subject). The therapeutically effective amount of the first therapy can be about 3.5 mg/kg (by weight of the subject).

The least one ligand of the compound or pharmaceutical composition can comprise an amino acid ligand substituted with one or more carboxamides.

In certain embodiments, administering comprises intramuscular injection of the compound, pharmaceutically acceptable salt, N-oxide, solvate, tautomer, or stereoisomer of the compound, or pharmaceutical composition into the subject, wherein the compound, pharmaceutically acceptable salt, N-oxide, solvate, tautomer, or stereoisomer of the compound, or pharmaceutical composition reaches maximum concentration in the subject at about or between 7-9 minutes post administration (such as at or between 7 minutes to about 9 minutes, at or between about 7 minutes to 9 minutes, or at or between 7 minutes to 9 minutes). In certain embodiments, after administered intramuscularly to the subject, the compound, pharmaceutically acceptable salt, N-oxide, solvate, tautomer, or stereoisomer of the compound, or pharmaceutical composition undergoes pH-induced isomerization resulting in a reduced cyanide scavenging rate as compared to a cyanide scavenging rate of the compound, pharmaceutically acceptable salt, N-oxide, solvate, tautomer, or stereoisomer of the compound, or pharmaceutical composition within 1 hour of administration to the subject.

Combination therapies for treating cyanide poisoning or cyanide exposure in a subject are also provided. In certain embodiments, a combination therapy for treating cyanide poisoning or cyanide exposure in a subject comprises administering to the subject: a therapeutically effective amount of cyanide chelating agent; and a therapeutically effective amount of an agent for ameliorating cyanide-induced oxidative stress in the subject. In certain embodiments, the cyanide chelating agent comprises a platinum(II) thioether comprising bidentate ligands.

The cyanide chelating agent can comprise a compound hereof, a pharmaceutically acceptable salt, N-oxide, solvate, tautomer, or stereoisomer of a compound hereof, a compound or pharmaceutically acceptable salt, N-oxide, solvate, tautomer, or stereoisomer having the following structure:

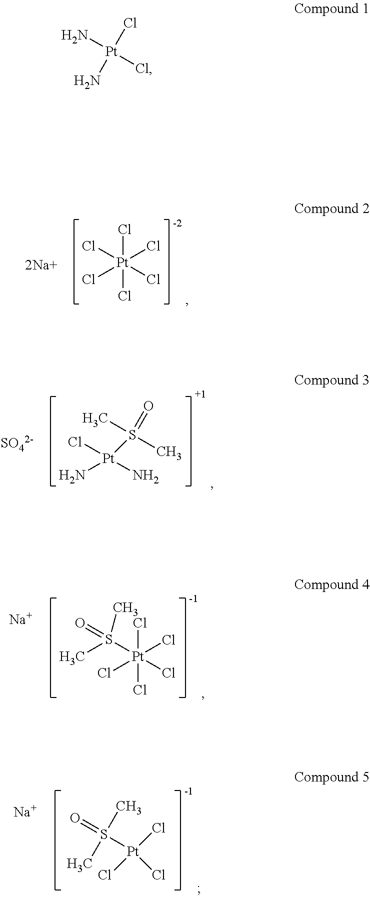

or
a pharmaceutical composition hereof.

The agent for ameliorating cyanide-induced oxidative stress in the subject can be glyoxylate or an analog or functional fragment thereof. The agent for ameliorating cyanide-induced oxidative stress in the subject can comprise a ketone or aldehyde compound selected from the group consisting of glyoxylate, glyceraldehyde, acetaldehyde, dihydroxyacetone, pyruvate, methylglyoxal, and alpha-ketoglutarate.

Kit for treating cyanide poisoning or cyanide exposure are also provided. In certain embodiments, a kit for treating cyanide poisoning or cyanide exposure comprise: a drug injection device comprising one or more fluid chambers prefilled with a first formulation comprising: a compound hereof, a pharmaceutically acceptable salt, N-oxide, solvate, tautomer, or stereoisomer of a compound hereof, a compound or pharmaceutically acceptable salt, N-oxide, solvate, tautomer, or stereoisomer having the following structure:

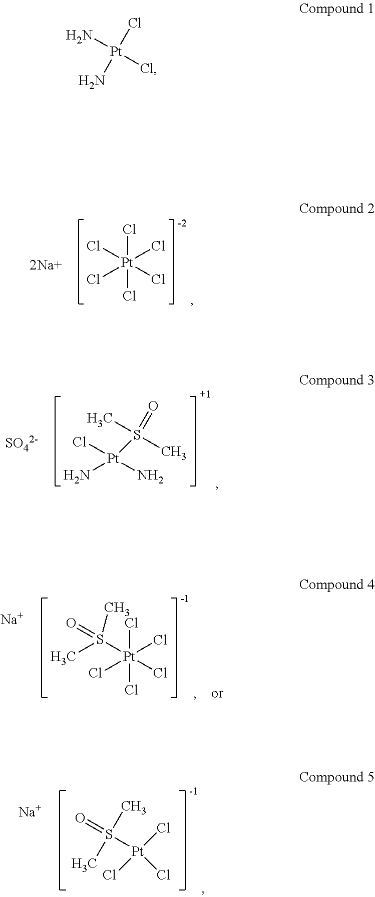

and
a pharmaceutically acceptable carrier and/or excipient.

The first formulation can comprise a targeted effective dose of the compound or pharmaceutically acceptable salt, N-oxide, solvate, tautomer, or stereoisomer for intramuscular injection. The prefilled fluid chamber can be a syringe or a cartridge. The formulation can have a pH value of 5 or less. The drug injection device can be an autoinjector or a hand-held injector.

The kit can further comprise one or more fluid chambers prefilled with a second formulation. The second formulation can comprise a therapeutically effective amount of an agent for ameliorating cyanide-induced oxidative stress in the subject and a pharmaceutically acceptable carrier and/or excipient. The agent for ameliorating cyanide-induced oxidative stress in the subject can be glyoxylate or an analog or functional fragment thereof.

### BRIEF DESCRIPTION OF THE FIGURES

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1 shows structures of the starting materials described herein (compounds 1-5) and dimethylsulfoxide (DMSO) complexes, as well as the structure of Pt(CN)₄²⁻ ion.

FIG. 2 depicts Scheme 1 and is a structural representation of Pt amine-sulfide containing complexes isolated and pharmacologically evaluated in the studies described herein, where the structures represent a major isomer assigned based upon the heteronuclear NMR spectra.

FIG. 3 shows x-ray fluorescence (XRF) spectra data of Pt(IV) and Pt(II) excitation core absorbances. The peaks associated with platinum were integrated against the manganese chloride internal standard at 6 keV.

FIG. 4 is a table showing percent weight (% w/w) composition of each platinum compound measured by XFR. Manganese chloride served as an internal standard for quantification.

FIG. 5 shows a table of countermeasures evaluated by monitoring reactions to cyanide in vitro, where the observed rate constants were acquired under pseudo-first-order conditions with 10 molar equivalence cyanide, product formation was monitored at 255 nm for Pt(CN)₄²⁻, cyanide was quantified using an ion-selective electrodes measuring the reduction of free cyanide after 10 minutes, the platinum content was adjusted by using the XRF values to correct concentrations, and high-performance liquid chromatography (HPLC) data were collected with 24 hours of preparation, equilibrium was presumed to be established at the time of collection (ID=compound number, and [symbol] =rate data for Pt(IV) compounds 2 and 4 were established from the disappearance of signal at 275 nm (a.u./min)).

FIGS. 6A-6D show reaction data between platinum and cyanide monitored by high-performance liquid chromatography (HPLC). FIG. 6A shows compound 6 and cyanide was quantified using a Resetek Ultra IBD (Restech Corporation, Houston, TX), FIG. 6B and FIG. 6C show photodiode arrays (200-300 nm) of compound 6 shown in FIG. 6A and Pt(CN)₄⁻² respectively, with FIG. 6C showing a strong charge transfer band at 260 nm consistent with the formation of Pt(CN)₄²⁻, and FIG. 6D showing data relating to compound 6 being titrated with 1-10 molar equivalences of cyanide.

FIGS. 6E and 6F show HPLC calibration data, with FIG. 6E showing a calibration curve of Pt(CN)4-2, where each point contains n=3 injections of standard material (error bars are small and not visible), and FIG. 6F showing a calibration curve of compound 6 where each point has n=3 injections of standard (error bars are small and not visible).

FIGS. 7A-7C show ¹⁹⁵Pt nuclear magnetic resonance (NMR) spectra of compound 6 hereof (51 mM in 200 mM NaPi 10% D₂O, pH 7.5 at 291.5 K titrated by cyanide, with FIG. 7A depicting before the addition of cyanide, FIG. 7B depicting after adding approximately 50 mM of cyanide, and FIG. 7C depicting after the addition of 200 mM (final concentration) of cyanide. The observed signals correspond to reactant (PtMet2) with Pt chemical shifts around −3650 ppm and final product [Pt(CN)₄²⁻] with Pt chemical shift at −4699 ppm.

FIG. 7D shows 1H NMR spectra data related to a competition reaction of compound 3 and compound 6 with KCN. Methyl signals at 2.08 ppm (*) and 2.67 (**) indicate the release of methionine and DMSO, respectively.

FIG. 8 shows data related to Pt amine-sulfide compound 11 reaction with potassium cyanide (KCN) monitored by ¹H NMR at 298K, with A showing 1 mM compound 11 alone in 50 mM NaPi, pH 7.5, 10% D₂O; B showing 1 mM KCN added; and C showing with 5 mM (final concentration) KCN added.

FIG. 9 shows ¹H NMR spectra of 1 mM compound 3 (Cisplatin-DMSO) without cyanide (bottom) and with 5 mM cyanide (top).

FIG. 10 shows ¹H NMR spectra of 1 mM compound 5 without cyanide (bottom) and with 5 mM cyanide (top).

FIG. 11 shows ¹H NMR spectra of 1 mM compound 7 without cyanide (bottom), with 1 mM cyanide (middle) and 5 mM cyanide (top).

FIG. 12 shows ¹H NMR spectra of 1 mM compound 8 without cyanide (bottom), with 1 mM cyanide (middle) and 5 mM cyanide (top).

FIG. 13 shows ¹H NMR spectra of 1 mM compound 9 without cyanide (bottom), with 1 mM cyanide (middle) and 5 mM cyanide (top).

FIG. 14 shows ¹H NMR spectra of 1 mM compound 10 without cyanide (bottom), with 1 mM cyanide (middle) and 5 mM cyanide (top).

FIG. 15 shows ¹H NMR spectra of 1 mM compound 11 without cyanide (bottom), with 1 mM cyanide (middle) and 5 mM cyanide (top).

FIG. 16 shows ¹H NMR spectra for a reaction of compound 9 (50 μM Pt(MetNH₂)₂Cl₂, pH~12) 50 minutes after preparation (bottom) and compound 9 with cyanide (50 μM Pt(MetNH₂)₂Cl₂, +500 μM KCN, pH~12) 60 minutes after preparation (top). The ¹H NMR spectra showed no observable change between the sample (50 μM compound 9) with cyanide and without cyanide, supporting lack of reaction within an hour of preparation.

FIG. 17 shows ¹³C NMR spectra of a reaction between compound 6 and cyanide in 95% rabbit serum/% D₂O, with A showing data from rabbit serum with 400 μM 13C-KCN, and B showing 100 μM of compound 6 added. The asterisk-labeled signal at 117 ppm was generated from KCN, as it exists mainly as HCN at neutral pH. The signal at 125 ppm was assigned to Pt(CN)₄²⁻ while satellites from ¹⁹⁵Pt coupling were not clearly observed presumably due to low signal-to-noise ratio and line-broadening. Semi-quantitation of Pt(CN)₄²⁻, based on the signal at 117 ppm (for 400 μM), was consistent with the expectation of full scavenging of cyanide by compound 6.

FIG. 18 shows graphical data related to cardiotoxicity testing of Pt compounds in zebrafish. (P<0.00005 for the dofetilide control)

FIG. 19 is a table describing a basic scheme for testing lethal exposure by inhalation in a mouse model.

FIG. 20A depicts the protocol of a non-lethal rabbit cyanide exposure study performed using compounds 3 and 6 hereof,

FIGS. 20B-20D show data resulting from the study described in FIG. 20A, wherein FIGS. 20B and 20C show data related to recovery of normal homeostatic levels of blood hemoglobin in rabbits treated intramuscularly (IM) with compound 3 and compound 6, respectively, after cyanide infusion, and FIG. 20D shows the results of a platinum blood plasma analysis determined by inductively coupled plasma mass spectrometry (ICP-MS) with the area under the curve (AUC) as the mean value of n=3 replicates calculated for the first 60 minutes after treatment (highest documented concentration (e.g., Cₘₐₓ) from a single injection paired with time observed (Tₘₐₓ)).

FIGS. 20E-20J are graphs depicting blood plasma concentrations of total platinum content collected by ICP-MS of rabbits dosed with either compound 3 or compound 6 in the non-lethal cyanide model described herein.

FIG. 21 shows graphs related to monitoring the stability of compound 4 by ¹⁹⁵Pt NMR and ¹H NMR.

FIG. 22 is mass spectrum data of compound 3 using the LTQ Orbitrap.

FIG. 23 is mass spectrum data of compound 4 using the LTQ Orbitrap.

FIG. 24 is mass spectrum data of compound 5 using the LTQ Orbitrap.

FIG. 25 is mass spectrum data of compound 6 using the LTQ Orbitrap.

FIG. 26 is mass spectrum data of compound 7 using the LTQ Orbitrap.

FIG. 27A is mass spectrum data of compound 8, and FIG. 27B is mass spectrum data of compound 8 using an Advion Expression spectrometer in electrospray ionization positive ion (top) and negative ion (bottom) modes.

FIG. 28 is mass spectrum data of compound 9 using the LTQ Orbitrap.

FIG. 29 is mass spectrum data of compound 10 using the LTQ Orbitrap.

FIG. 30 is mass spectrum data of compound 11 using the LTQ Orbitrap.

FIG. 31 is ¹H NMR spectra data (500 MHz) for a fresh solution of 15.6 mg compound 6 in 500 μL of 90 mM NaPi, pH 8.0 (final pH~5.7), 10% D₂0 at 292K.

FIG. 32 shows the structure of Pt(II) compounds 1′-4′ described herein.

FIGS. 33A and 33B show osmolality curves for compound 6′ (+2NaCl) (FIG. 33A) and compound 9 (+2NaCl) (FIG. 33B). The data was taken from a single stock solution and subsequently diluted with purified water.

FIGS. 34A and 34B show, as representative examples, the reaction of compound 13 (+2NaCl) (FIG. 34A) or compound 6 (+2NaCl) (FIG. 34B) with 1:40 Pt:CN⁻ in purified water. The samples were also incubated for a minimum of 24 hours and Pt(CN)₄²⁻ was quantified by UV-Vis once the signal was stable and consistent with Day 0 kinetics.

FIGS. 34C and 34D show, respectively, as representative examples, the reaction of compound 13 (+NaCl) (FIG. 34C (bars representing 48 hours)) and compound 6 (+NaCl) (FIG. 34D) with KCN in 1:5, 1:10, 1:80 Pt:CN in purified water.

FIG. 35 shows data related to the UV-Vis spectra of compound 13 after adding 100× molar equivalences of NaOH to monitor spectral changes over 710 minutes.

FIG. 36A is graphical data from combining compound 9 with 50 molar equivalences NaOH and waiting 240 minutes, with peak development at 245 nm.

FIG. 36B is graphical data from combining compound 9 with varying amounts of NaOH, with the signal at 245 nm increasing with the increasing NaOH concentration.

FIG. 36C is spectra analysis of compound 9, when incubated at room temperature in pH 6.8 phosphate buffer. Compound 9 showed a new form labeled as Peak II by HPLC after about 175 minutes (blue trace (bottom line)) is compound 9 in purified water with detection absorbance at 220 nm.

FIG. 36D is a graph of absorption changes for compound 9 in the presence of KCN, monitored at 245 nm. Compound 9 was prepared in a pH of 7.26 phosphate buffer at 18.8° C. Data shown is a result of the absorbance increase at 245 nm. Scans were taken every 0.1 minutes, 1 minute, and 30 minutes, respectively, throughout the course of the analysis. Data fits linearly with a semi log plot, suggesting the process follows first order kinetics. Half-life of appearance for these conditions is 2.6 hours with R²=0.9962.

FIGS. 37A and 37B show graphs of the time-dependent changes of compound 13 in phosphate buffer, pH 7.3 at 19° C. (FIG. 37A) and 37° C. (FIG. 37B) when allowed to react with KCN. Data were acquired by monitoring signal at 241 nm.

FIGS. 38A and 38B show ¹H NMR spectra data demonstrating the transformation of compound 9 at pH 7.15 at room temperature (day 0 labeled B, and day 3 labeled A). FIG. 38A shows a downfield region, for amino and amide NH's, supporting their subtle changes over time.

FIG. 38B shows upfield regions showing changes for the ligand sidechain.

FIG. 39 is ¹H NMR spectra data monitoring changes in free —SMe for compound 9 when incubated in pH 7 phosphate buffer for 3 weeks with the addition of HCl to adjust the solution pH to approximately 2.5.

FIG. 40 shows data from compound 9 being incubated in pH 6.8 phosphate buffer for 3 days at room temperature. Data shows predominately “Peak II” at T=0 slowing reacted with 2 mM of compound 9 and 8 mM KCN to product Pt(CN)₄⁻².

FIGS. 41A-41D are HPLC stability results for compounds 6, 9, 12, and 13, respectively, after each compound was reacted with 4 mole equivalences of KCN for 10 minutes prior to injection into the HPLC to quantify Pt(CN)₄²⁻ produced.

FIG. 42 is HPLC stability results for compound 6 reacted with 4 mole equivalences of KCN for 10 minutes prior to injection in the HPLC to quantify Pt(CN)₄²⁻ produced to monitor reactivity for 42 days. Data shown for time point is the average of 3 replicates.

FIG. 43 is a titration curve of ~5 μmoles of compound 6 with 5 mM NaOH after freshly prepared in water versus aged at room temperature for 7 days.

FIG. 44 is a titration curve of ~5 μmoles of compound 9 with 5 mM NaOH, which shows a lack of a clear equivalence point indicating a more complex chemical process than titration of an ionizable group.

FIG. 45 shows zebrafish data that represents the antidote activity (EC₁₀₀) of each compound formulation (compounds 6, 9, 12, and 13) with aqueous conditions. Each result is the concentration of platinum necessary for 100% (n=5) survival in the presence of 100 μM KCN, a concentration that results in mortality after 1 hour in the control groups. Reported survival times were measured 4 hours after cyanide exposure, and compounds were prepared 3-7 days prior to use.

FIGS. 46A and 46B is graphical data from a study were rats were dosed with compound 6a via intraperitoneal (IP) injection using various dosages. FIG. 46A shows mean blood urea nitrogen (BUN) concentrations for the male and female cohorts, both showing significant (p<0.0005) signs of acute kidney injury (AKI) after 5 days at 218 μmole/kg (42.5 mg Pt/kg). FIG. 46B shows mean creatine (CREA) concentrations for the male and female cohorts, both showing significant levels as compared to the vehicle after day 1 and day 5 (p<0.05 and p<0.0005, respectively). The analysis was carried out using an ordinary two-way ANOVA with a Ŝidák multiple comparisons comparing the cell means regardless of rows and columns.

FIGS. 47A and 47B are graphs showing BUN (FIG. 47A) and CREA (FIG. 47B) levels in a cohort of rats after treatment with compound 6 to assess the role, if any, of osmotic pressure. Two formulations of compound 6 were tested, as well as a vehicle for comparison purposes.

FIG. 48 is a graph of body weight change of rats dosed with increasing amounts of compound 6α.

FIGS. 49A-49C illustrate the changes observed in the levels of AKI markers BUN, CREA, and PHOS in response to the 5× dose of each compound 6, 9, 12, and 13, and show toxicity as indicated in observed at the highest dose of 218 μmole/kg (42.5 mg Pt/kg). Compound 9 was assessed at two pH values. The analysis was carried out using an ordinary two-way ANOVA with a Ŝidák multiple comparisons comparing the cell means regardless of rows and columns.

FIGS. 50A and 50B are graphs showing BUN and glucose (GLU) levels (FIG. 50A or 50B, respectively) as example markers of kidney dysfunction. The analysis was carried out using an ordinary two-way ANOVA with a Ŝidák multiple comparisons comparing the cell means regardless of rows and columns.

FIG. 51 is a profile of total platinum in plasma concentration versus time, as measured by ICP-MS in rats for compounds 6 and 9. The expanded earlier concentration versus time profile (inset) illustrates the apparent differences in distribution early on between the two compounds.

FIG. 52 shows the structures of various ligands for incorporation into the compounds hereof.

FIG. 53 shows a workflow and performance metrics that can be applicable to selecting and advancing new ligands and new models.

FIG. 54 shows a workflow and performance metrics that can be applicable to formulating Pt(II)-glyoxylate combinations.

FIG. 55 is a graph of data from a combination therapy comprising a Pt(II) cyanide scavenger and glyoxylate, wherein the combined therapy rescued lethal zebrafish model following challenge (glyoxylate EC₁₀₀=32 μM; PCP EC₁₀₀=62 μM; PCP @15 μM plus 8 μM glyoxylate (100% survival) PCP @32 μM plus 4 μM glyoxylate (100% survival).

FIG. 56 is example 13C NMR data that enables measurement of cyanohydrin formation (pyruvate in this case). The Kd (μM) for each metabolite is: glyoxylate 1.7, glyceraldehyde 6, alpha-ketoglutarate 80, pyruvate 90, dihyroxyacetone 200, and glucose>3000.

FIG. 57 is a graph of the survival rate in cyanide-poisoned swine following IM administration of glyoxylate (glyoxylate improves survival). Kaplan-Meier plot of survival in control (n=8; labeled A) versus glyoxylate (n=6; labeled B) treated animals (p<0.001).

FIGS. 58A and 58B are graphs of plasma biomarker data that reflect that intracellular and extracellular redox balance improved in glyoxylate-treated swine. In swine treated with cyanide (gray box) and subsequently, at t=0, 10 mg/kg glyoxylate was administered intramuscularly (labeled A; n=6) or 20 mg/kg hexachloroplatinate was administered intramuscularly (labeled B; n=3), serial plasma samples were taken to measure the 1) ratio of lactate:pyruvate, 2) a circulating marker that is in near equilibrium with cellular NADH:NAD+, and 2) a ratio of cysteine:cystine, a plasma biomarker of extra cellular oxidative stress.

FIG. 59 shows a workflow and performance metrics that can be applicable to an evaluation in the swine model described herein.

FIG. 60 shows preliminary data examples of Pt blood levels post IM injection of the stated dose in the swine model. The agent (Met₂Pt) was delivered in 6 mL of formulation. These data indicate the feasibility of meeting the stated performance criteria.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

Cyanide poses a risk to human health as an agent in at least chemical warfare, suicide, occupational exposure, and smoke inhalation. Cyanide potently inhibits cytochrome c oxidase and potentially other metabolic enzymes, thereby unleashing a cascade of metabolic perturbations. More specifically, cyanide inhibits complex IV of the respiratory chain by binding the ferric ion on cytochrome a3. Fixation of cyanide on cytochrome c oxidase prevents its reoxidation by oxygen and thereby causes a backup of electrons on the electron transport chain. Complex I is subsequently stuck in a reduced state and therefore cannot reduce nicotinamide adenine dinucleotide (NADH) to regenerate the NAD+ that is required for the tricarboxylic acid (TCA) cycle. As NADH/NAD+ ratio increases, negative feedback inhibition on the TCA cycle ensues. Electron transport and oxidative phosphorylation can be halted, with resultant shifts of cellular metabolism from aerobic to anaerobic.

If not reversed, the cessation of aerobic metabolism can cause a fatal deficit in oxygen consumption. As such, cyanide is most toxic to organs with high metabolic requirements, such as the brain and heart. Milligram quanitites of cyanide can cause convulsions, seizures, cardiovascular collapse, and death within minutes of exposure, while lower doses can cause a spectrum of debilitating, long-lasting pathologies including a Parkinson-like syndrome due to irreversible neuron death in select brain areas.

The body has a natural defense mechanism to remove cyanide. The pathway of cyanide detoxification is through the sulfur transferase rhodanese, resulting in the sulfuration of cyanide to form the less toxic compound thiocyanate, which is excreted in the urine.

Though there are some conventional antidotes available for cyanide poisoning, their formulation and mode of action require intravenous (IV) infusion in a hospital setting with a single dose taking at least 15 to 20 minutes to administer, which is not amendable to a mass casualty event or for field operations.

Additionally, like other metallodrugs, platinum-based drug products have known nephrotoxicity, ototoxicity, cardiotoxicity, and peripheral neuropathy risks. Oun et al., The side effects of platinum-based chemotherapy drugs: a review for chemists, Dalton Transactions 47: 6645-6653 (2018). Acute kidney injury (AKI) is commonly encountered in patients receiving cisplatin, which is highly dependent on dose, dose frequency, and cumulative dose. Miller et al., Mechanisms of cisplatin nephrotoxicity, Toxins 2: 2490-2518 (2010). Co-administration of methionine and other sulfur-containing agents has also been demonstrated to modulate cisplatin AKI. Jones et al., Thiol and thioether suppression of cis-platinum-induced nephrotoxicity in rats bear the Walker 256 carcinosarcoma, Anticancer Research 9: 1937-1941 (1989); Jones et al., Thioether suppression of cisplatin nephrotoxicity in the rat, Anticancer Research 11: 449-453 (1991). The modulation of platinum-induced AKI from cisplatin is hypothesized to be due to reduced reactions with thiol-containing amino acids, peptides, and proteins in the cytosol. Stankovic et al., Antioxidant supplementation in the treatment of neurotoxicity induced by platinum-based chemotherapeutics—a review, IJMS 21: 7753 (2020).

In previous work, in vivo screens of chemical agents that rescue zebrafish from toxic levels of cyanide exposure led to the discovery of platinum-based complexes. The proposed mechanism of action was based on the known interaction of platinum with cyanide to form stable complexes. A list of 36 organoplatinum complexes including drugs such as cisplatin and oxaplatin were tested. Each sample required heating in dimethylsulfoxide (DMSO) before dilution in phosphate-buffered saline to reveal meaningful rescue activity. Interestingly, the cyanide protective activities of these materials all share the common requirement that samples have to be pre-treated in DMSO before dilution and use for the rescue of cyanide.

DMSO reactions with platinum II (Pt(II))-based therapeutic agents are well known. The cisplatin formulated with DMSO, when administered via the intraperitoneal (TP) route has shown to rescue mice treated with lethal doses of cyanide. Using this formulation approach, hexachloroplatinate (HCP) was further tested in rabbit and pig models of cyanide toxicity by IP and by IM administration to demonstrate the efficacy of survival from lethal cyanide exposures. Evidence for formation of platinum cyanates in circulation and reversal of metabolic blockade of Krebs' cycle was observed. Both DMSO-treated cisplatin and HCP reversed symptoms of cyanide-induced oxyhemoglobin monitored in rabbit circulation to substantiate the reduction in toxic substance levels. These studies established the necessary proof of concept that cyanide-reactive Pt complexes can display antidote properties in these animal models.

The prior use of platinum in pharmaceuticals in clinical settings offers some guidance to the design of practical cyanide countermeasures. Cisplatin, oxaliplatin, and carboplatin are widely used anticancer agents administered only by IV infusion. The dose-limiting toxicity of cisplatin is 20-fold higher than HCP, while the solubility is 33-fold lower than HCP. Interestingly, the use of DMSO with cisplatin and related drugs is known to reduce the antitumor potency and toxic side effects. However, some conventional compounds are associated with dose-limiting toxicity.

Furthermore, FDA-approved countermeasures that can act by directly binding cyanide, i.e., hydroxycobalamin, do not ameliorate the biological and metabolic damage caused by cyanide. On the other hand, glyoxylate or other metabolic agents that mitigate the effects of cyanide do not eliminate cyanide from the body.

In view of the above, provided are compounds and pharmaceutically acceptable salts thereof that are soluble, exhibit reduced toxicity (e.g., are non-toxic) as compared to conventional cyanide antidotes, efficacious, and amendable for mass distribution and administration to affected individuals to prevent morbidity and mortality resulting from exposure to cyanide. In certain embodiments, the compounds hereof are therapeutically effective when administered intramuscularly (IM). In certain embodiments, the compounds exhibit a time and/or pH dependence on cyanide reactivity both in vitro and in vivo efficacy.

In addition, in certain embodiments, the platinum-based compound comprises amino sulfide-containing bidentate ligands to direct reactions of the Pt(II) complexes with cyanide. As used herein, a “bidentate ligand” means a molecule or ion that donates electron pairs to the core metal ion of the compound, forming coordinate covalent bonds. Such compounds have been found to increase the rate of addition to produce tetracyanoplatinate(II). Importantly, this reactivity directly translates to enhancements in the capacity of Pt(II) to rescue cyanide toxicity in at least zebrafish, mouse and rabbit models. The variation in ligand composition opens a new avenue for further lead candidate identification for next generation cyanide countermeasures.

Still further, combinations are provided that combine a chelating agent hereof (e.g., a Pt(II)-complex described herein) with the administration of a metabolic modulator to address not only cyanide capture, but also amelioration of cyanide-induced oxidative stress in the body.

### Compounds

The compounds hereof are cyanide scavengers and capable of activating and directly binding cyanide anions. The compounds comprise coordination complexes having at least the following components: (1) a core metallic atom or ion that is the coordination center; and (2) one or more bidentate ligands (comprising ligands L₁ or L₂ as described in connection with Formulae (I)-(III)) bonded to the core metallic atom or ion.

The core metallic atom or ion can be a platinum atom (Pt) or Pt ion. The core can be charged (e.g., the core metallic atom can be a positively charged metallic atom). The core can be charged Pt. The core can be a platinum(II) (Pt(II)) atom. The term “platinum(II)” or “Pt(II)” means a platinum ion having a +2 charge. The Pt(II) ion is a hexa-coordinate and bound to only neutral (uncharged) ligands. The term “ligand” means a molecule that engages in a coordination bond to the Pt(II) ion. The term “uncharged” indicates that the ligand is not charged (i.e., anionic or cationic) either inherently in the atoms making the core structure or as groups appended to the core structure. In certain embodiments, two of the six coordination sites of the Pt(II) are occupied by at least one bidentate, bicylic ligand. The ability of bidentate ligands to bridge and/or form two bonds with the core metal of the compound can increase the stability and coordination of the complex.

Unlike cobalt (the backbone of conventional hydroxocobalamin, cobinamide (a naturally occurring precursor to cobalamin (Vitamin B12), and Co(III) porphyrins and Schiff base complexes) which involves the direct binding of cyanide in a 1:1 stoichiometric ratio, Pt(II) can bind 4 more equivalences of cyanide, thus providing increased efficacy as compared to conventional compounds. Nath et al., Cisplatin analogs confer protection against cyanide poisoning, Cell Chemical Biology, 24: 565-575 (2017); Cronican et al., A comparison of the cyanide-scavenging capabilities of some cobalt-containing complexes in mice, Chemical Research Toxicology 31: 259-268 (2018); Chan et al., Cobinamide is superior to other treatments in a mouse model of cyanide poisoning, Clinical Toxicology (Philia) 48: 709-717 (2010). More specifically, cobalt scavengers bind only 1 or 2 molar equivalence of cyanide. Lopez-Manzano et al., Cyanide scavenging by a cobalt Schiff-Base macrocycle: a cost-effective alternative to Corrinoids, Chemical Research Toxicology (Philia) 29: 1011-1019 (2016).

When administered via IM injection, the Pt(II) complexes hereof have proven efficacious in a mouse cyanide inhalation model as low as 0.090 mmol/kg of platinum. Behymer et al., Identification of platinum(II) sulfide complexes suitable as intramuscular cyanide countermeasures, Chemical Research in Toxicology 35(11): 1983-1996 (2022). Accordingly, in certain embodiments, the Pt(II)-based complexes provide an IM-available agent with high efficacy per molar equivalence of metal, while concurrently reducing the overall molecular weight as compared to conventional cobalt-based scavengers. Id.

At least one or more of the ligands of the compound is a leaving group (e.g., groups that can be replaced/displaced by cyanide). In certain embodiments, at least three of the ligands are leaving groups to the extent the non-leaving ligands do not interfere with the cyanide reaction with the Pt core or make toxic complexes with Pt(II). In certain embodiments, four ligands are leaving groups. The concept of ligand release upon cyanide addition can be leveraged to provide additional beneficial effects without adding toxicity burdens.

One or more of the ligands can comprise a metabolic modulator (i.e., capable of modulating the reactivity of the platinum center of the compound to promote reaction with hydrogen cyanide). The selected ligand structure can be used to modulate or tune the reactivity of the platinum center of the compound for reaction with hydrogen cyanide. In certain embodiments, this can promote selectivity for cyanide while de-risking the potential for toxicity (e.g., rental toxicity). For example and without limitation, a ligand can comprise one or more sulfurs (e.g., sulfides) to promote sulfur-directing activation of Pt(II) towards cyanide addition and/or ligand displacement in vivo. In certain embodiments, at least two of the ligands comprise a sulfide such that the compound comprises a disulfide.

Table 1 lists several non-limiting examples of platinum compounds that are tunable (i.e., can be used to modulate and/or promote selectivity for cyanide) as described herein.

TABLE 1
Pt-compound protection from cyanide (EC₁₀₀ values
derived from in vivo data from ligands studied in the zebrafish models
described herein, with all indicating efficacy except Pt(taurine)₂(S₂O₃)₂)

| Pt-Compound | Diluent | EC100 (μM) |
|---|---|---|
| HCP-AKN | DMSO | 62 |
| cisplatin-AKN | DMSO | 15 |
| Pt(taurine)₂(S₂O₃)₂ | H₂O | >250 |
| (SAllylCys)₂Pt | H₂O | 7.8 |
| (SMePenicillamine)₂P | H₂O | 7.8* |
| (cilastatin)₂Pt | H₂O | 7.8* |
| (bridged-Met2)Pt | H₂O | 7.8 |
| MetPt(taurine)₂ | H₂O | 7.8 |

*50% survival @3.4 μM

In certain embodiments, a ligand is selected for inclusion in the bidentate ligand and/or compound if it has one or more of the following characteristics: the ligand is commonly well-tolerated in vivo as a free ligand, enhances affinity for Pt(II) and stability in the biological matrix, and imparts some renal protective effects in rat models of cisplatin toxicities or similar models (e.g., similar to cilastatin and taurine).

The bidentate ligands of the compound or pharmaceutically acceptable salt can be the same, different, or a mixture thereof. A ligand can comprise a molecule that complexes with the platinum core of the compound (e.g., to form the bidentate ligand). The functional groups in the ligands reside in a bridging relationships to enable bidentate orientation binding to the Pt(II).

The compound can comprise bicylic bidentate ligands bonded to the platinum core. The bidentate ligands of a compound can independently 5- or 6-membered bidentate ligands bonded to the platinum. At least one bidentate ligand can comprise a 5-membered bidentate ligand bonded to the platinum core. At least one bidentate ligand can comprise a 6-membered bidentate ligand bonded to the platinum core. In certain embodiments, the bidentate ligands of the compound independently comprise 5- or 6-membered bidentate ligands, and at least one of the bidentate ligands comprises a carboxylate or carboxamide substituent. In such embodiments, the compounds can be trans-directing sulfur ligands on Pt(II) that are efficacious in vivo when delivered intramuscularly (IM).

At least one or two of the ligands of a bidentate ligand can be a thioether (e.g., at least one ligand can comprise an amino sulfide). At least one thioester ligand can comprise an amino sulfide. In certain embodiments, the ligand(s) comprising the thioether can further comprise an alkyl, a carboxyamide, an amine, an amino sulfide, a carboxylate, a carboxyester, a carbonyl, or a combination of any of the foregoing to the extent the thioether comprises a carbon-bound sulfide and the ligand does not interfere with the cyanide-Pt core cyanide reaction or make toxic complexes with Pt(II).

The ligand can comprise an amino acid. The ligand can comprise methionine. The ligand can comprise methionine sulfoxide. The ligand can comprise N-acetyl-L-methionine (Ac-methionine). The ligand can comprise S-methyl-L-cysteine (SMeCys). The ligand can comprise reduced glutathione. The ligand can comprise SMeCys sulfoxide. The ligand can comprise S-methylated glutathione. The ligand can comprise any carbon-bound sulfide.

In certain embodiments, a first ligand of a bidentate ligand comprises a sulfide and a second ligand of the bidentate ligand comprises an amide.

At least one of the ligands can comprise a carboxyamide. At least one of the ligands can comprise an amine. The amine can be an amino sulfide. In certain embodiments, at least one of the ligands comprises a carboxylate or a carbonyl. A ligand can comprise a carboxyester.

In certain embodiments, one or more of the ligands can leverage the protective effects via the co-administration of ketone and aldehyde compounds such as glyoxylate, glyceraldehyde, acetaldehyde, dihydroxyacetone, pyruvate, methylglyoxal, and alpha-ketoglutarate. Nielson et al., Glyoxylate protects against cyanide toxicity through metabolic modulation, Scientific Reports 12: 4982 (2022). The cytochrome c oxidase levels were rapidly restored in rabbits treated with glyoxylate as compared to alpha-ketoglutarate. Additional mechanistic studies reveal that the glyoxylate rescue effect is dependent upon lactate dehydrogenase activity with evidence that glyoxylate is being rapidly oxidized to form oxalate. Overall, the cyanide chelation mechanism of action is a component of glyoxylate cyanide protection, but an additional metabolic contribution appears to play a role in its enhanced efficacy.

The observation for alpha-ketoglutarate was not surprising in light of the previous work in the field. This agent's mechanism of action has been attributed primarily to the chelation of cyanide in the form of a reversible covalent bond to form a cyanohydrin. While the glyoxylate dosing is high on a per mole of cyanide basis, the efficacy is >10-fold improved. More importantly, this boost in effectiveness translated to in vivo efficacy with striking results in both the mouse inhalation and rabbit models when administered glyoxylate via IM. Id. The cytochrome c oxidase levels were rapidly restored in rabbits.

One or more of the ligands can comprise a ligand such as methionine that can produce S,N-chelates with platinum and can exist in solution as multiple isomers. The ligand(s) of each bidentate ligand can be selected to influence the assay method through which the compound scavenges cyanide and, as such, influence antidotal efficacy of the compound. Behymer et al. (2022), supra.

Additionally, ligand selection can take into account that certain structures exhibit pH dependencies with respect to cyanide reactivity. The association rate to platinum by amines, for example, increases at a higher pH, with losing a proton to encourage Pt—N bond formation. Summa et al., Thermodynamic and kinetic studies on reactions of Pt(II) complexes with biologically relevant nucleophiles, Inorganic Chem 45: 2948-2959 (2006); Appleton et al., S,O- versus S,N-chelation in the reactions of the cis-diamminediaquaplatinum(II) cation with methionine and S-methylcysteine, Inorganic Chem 27: 130-137 (1988); Appleton et al., Nitrogen-15 and platinum-195 NMR spectra of platinum ammine complexes: trans- and cis-influence series based on platinum-195-nitrogen-15 coupling constants and nitrogen-15 chemical shifts, Inorganic Chemistry 24: 4685-4693 (1985). This can be leveraged to facilitate safety of the compound and prevent toxicity (e.g., dose-limiting toxicity).

The ligand can be substituted. The ligand can be unsubstituted. In certain embodiments, the ligand is substituted with a carboxyamide. In certain embodiments, the ligand is substituted with a carboxylate and/or a carboxyester. The ligand can be substituted with one or more amidated carboxylates.

A ligand can be optionally substituted. “Optionally substituted” means unsubstituted or substituted. As used herein, the term “substituted” means that a hydrogen atom is removed and replaced by a substituent. It is to be understood that the substitution at a given atom is limited by valency.

At least one of the ligands can comprise a methyl thioether group. At least one of the thioether ligands can comprise methionine or S-methylcysteine optionally comprising one or more amidated carboxylates.

At least one of the ligands can be substituted with a halogen (i.e., such that the halogen is not in direct coordination of halogen to the Pt core). A “halogen” by itself or as part of another substituent means, unless otherwise stated, a fluorine (Fl), a chlorine (Cl), a bromine (Br), or an iodine (I) atom. For example, a halogen can be bound to a carbon in the ligand bridge of the bidentate ligand.

The ligands (i.e., leaving groups and/or those that remain conjugated following binding) can be selected to enhance the reactivity of the metal center. For example, and without limitation, at least one of the ligands can be a methyl thioether group.

In certain embodiments, a ligand of the compound is substituted with carboxamide or an ester amide. The structure and/or size of certain non-ligating substituents on a ligand of the compound has the potential to affect the reactivity of the platinum core with respect to cyanide anions.

In certain embodiments, the compound comprises a complex comprising: (1) a core metallic atom; (2) at least one ligand (L₁ and/or L₂) coordinated to the core metallic atom that are leaving groups, and (3) one or two ligands (L₁ and/or L₂) coordinated to the core metallic atom that remain conjugated to the positively charged metallic atom following binding of a cyanide atom (e.g., groups that remain conjugated to the metal in the presence of cyanide).

In some embodiments, the compound has a structure of Formula (I):

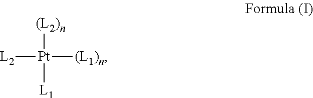

or a pharmaceutically acceptable salt, N-oxide, solvate, tautomer, or stereoisomer thereof, wherein:
Pt is platinum (e.g., resides in an oxidation state of Pt(II));
each L₁ and L₂ is a ligand, wherein each L₁ forms a bidentate ligand and each L₂ forms a bidentate ligand, each bidentate ligand comprising an N, an S, or both an N and an S coordinated to the Pt, wherein at least one of the ligands is a leaving group and at least two of the ligands that are directly bonded to the Pt(II) each independently comprise a thioether comprising an alkyl, a carboxyamide, an amine, an amino sulfide, a carboxylate, a carboxyester, a carbonyl, or a combination of any of the foregoing; and
each n is independently −1-5.

In certain embodiments of Formula (I), Pt is platinum(II).

The compound can have a structure of Formula (II):

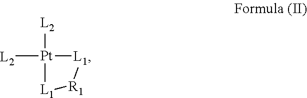

or is a pharmaceutically acceptable salt, N-oxide, solvate, tautomer, or stereoisomer thereof, wherein:
Pt is platinum (e.g., resides in an oxidation state of Pt(II));
each L₁ and L₂ is a ligand, wherein each L₁ forms a bidentate ligand and each L₂ forms a bidentate ligand, each a bidentate ligand comprising an N, an S, or both an N and an S coordinated to the platinum, wherein at least one of the ligands is a leaving group and at least two of the ligands that are directly bonded to the platinum each independently comprise a thioether comprising an alkyl, a carboxyamide, an amine, an amino sulfide, a carboxylate, a carboxyester, a carbonyl, or a combination of any of the foregoing;
R₁ is selected from a group consisting of C₁₋₃ alkyl, C₁₋₃ haloalkyl, C₂₋₄ alkenyl, C₂₋₄ alkynyl, and C₆₋₁₀ aryl, or absent; and
each n is independently −1-5.

In certain embodiments of Formula (II), Pt is platinum(II).

The compound can have a structure of Formula (III):

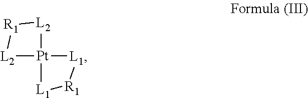

or is a pharmaceutically acceptable salt, N-oxide, solvate, tautomer, or stereoisomer thereof, wherein:
Pt is platinum (e.g., resides in an oxidation state of Pt(II));
each L₁ and L₂ is a ligand, wherein each L₁ forms a bidentate ligand and each L₂ forms a bidentate ligand, each a bidentate ligand comprising an N, an S, or both an N and an S coordinated to the platinum, wherein at least one of the ligands is a leaving group and at least two of the ligands that are directly bonded to the platinum(II) comprise a thioether comprising an alkyl, a carboxyamide, an amine, an amino sulfide, a carboxylate, a carboxyester, a carbonyl, or a combination of any of the foregoing;
each R₁ is independently absent or independently selected from a group consisting of C₁₋₃ alkyl, C₁₋₃ haloalkyl, C₂₋₄ alkenyl, C₂₋₄ alkynyl, and C₆₋₁₀ aryl, or absent; and
n is independently −1-5.

In certain embodiments of Formula (III), Pt is platinum(II).

Where R₁ is wholly absent in Formula (III), it will be appreciated that Formula (III) equates with Formula (I). Where one R₁ is absent in Formula (III), that Formula (III) can equate with Formula (II).

The ligands (L₁ and L₂) of Formula (I)-(III) can be any of the ligands described herein. One or more ligands of Formula (I)-(III) can be or comprise a sulfide. In certain embodiments, at least two ligands of Formula (I)-(III) are a sulfide (e.g., the compound comprises a disulfide).

The ligands L₁ and L₂ (and, thus, the bidentate ligands) of the compound or pharmaceutically acceptable salt, N-oxide, solvate, tautomer, or stereoisomer can be the same, different, or a mixture thereof.

At least one of the ligands (L₁ and/or L₂) of Formulae (I)-(III) can comprise a carboxyamide or a carboxyester. At least one of the ligands can comprise an amine. The amine can be an amino sulfide. In certain embodiments, at least one of the ligands comprises a carboxylate or a carbonyl.

In certain embodiments, a first L₁ comprises a sulfide ligand and a second L₁ comprises an amide ligand. Additionally or alternatively, a first L₂ can comprise a sulfide and a second L₂ can comprise an amide.

The compounds of Formulae (II) and (III) can comprise one or more spacers (R₁). A spacer (R₁) can be a C₁₋₃ alkyl. The spacer can be branched. The spacer can be non-branched or straight-chained (e.g., straight-chained alkyl groups). Some examples of straight-chained alkyl groups include methyl, ethyl, n-propyl, n-butyl, n-pentyl, and n-hexyl groups. The spacer can comprise an alkyl group, an alkenyl group, a haloalkyl group, an alkynyl group, or an aryl group. The spacer can comprise a peptide, a peptidoglycan, a polyethylene glycol (PEG) linker, a PEG derivative linker, or a combination of two or more of the foregoing. In some embodiments, the PEG linker can comprise (—CH₂CH₂—O—)ₙ, where n is an integer between and including 1 and 16. In certain embodiments, n of the PEG linker is an integer between and including 1 and 4. In certain embodiments, the spacer is an optionally substituted C₁₋₃ alkyl. In certain embodiments, the spacer is an optionally substituted C₁₋₃ haloalkyl. In certain embodiments, the spacer is an optionally substituted C₂₋₄ alkenyl. In certain embodiments, the spacer is an optionally substituted C₂₋₄ alkynyl. In certain embodiments, the spacer is an optionally substituted C₆₋₁₀ aryl.

The spacer can comprise a C₂-C₁₈ alkyl group, a peptide fragment, or a peptidoglycan fragment. As used herein, the term “fragment” means a molecule that has been modified to allow linking in the compound either as monovalent linking or bivalent linking, such as in the case of R₁ in Formulae (II) or (III). The use of the term “fragment” does not require that from a synthetic perspective; the molecule it refers to is made in the preparation of the compound. It is a description for moiety within the compound, regardless of how made.

In certain embodiments, the spacer (R₁) is absent.

The spacer can be designed to tune the reactivity of the compound to cyanide anions.

In certain embodiments, the compound has one of the following structures:

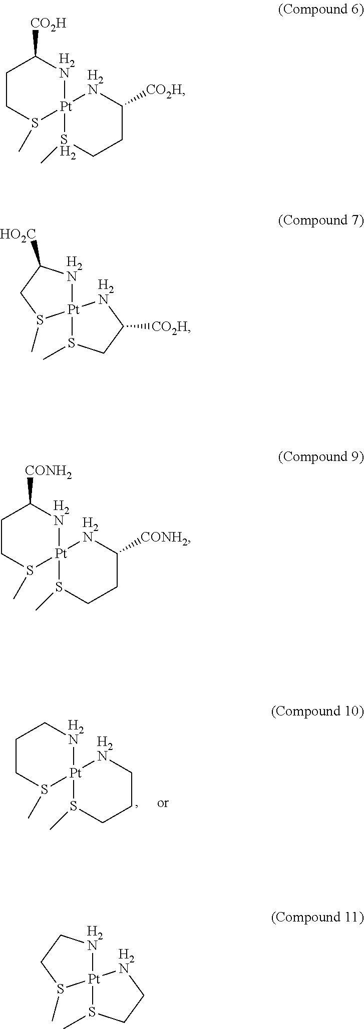

or is a pharmaceutically acceptable salt, N-oxide, solvate, tautomer, or stereoisomer thereof (e.g., a pharmaceutically acceptable salt, N-oxide, solvate, tautomer, or stereoisomer of any one of the preceding structures).

The compound can have one of the following structures:

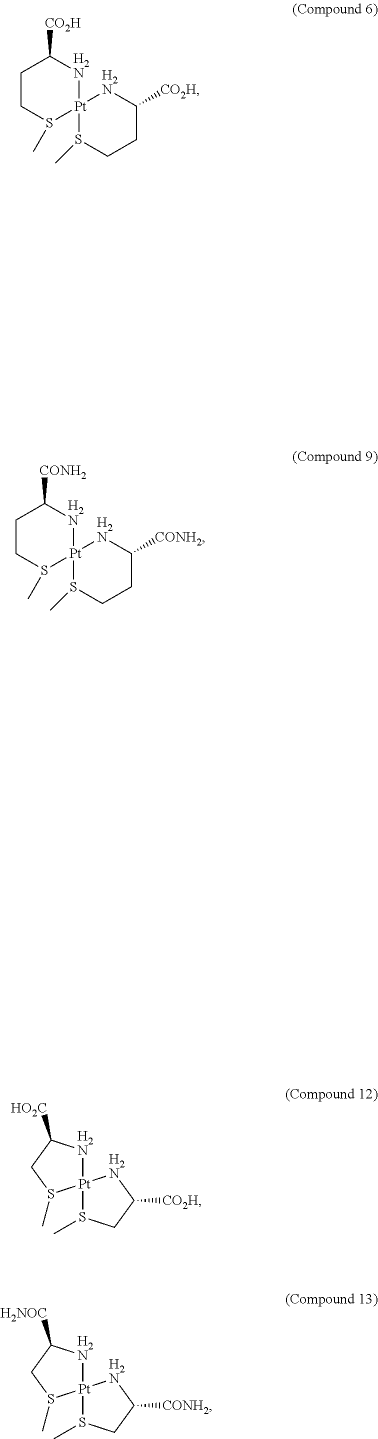

or can be a pharmaceutically acceptable salt, N-oxide, solvate, tautomer, or stereoisomer of any of the foregoing structures.

In certain embodiments, the compound comprises HCP-AKN, cisplatin-AKN, (SallylCys)₂Pt, (SMePenicillamine)₂P, (cilastatin)₂Pt, or (bridged-Met₂)Pt, MetPt(taurine)₂.

In certain embodiments, the compound comprises the following structure or is a pharmaceutically acceptable salt, N-oxide, solvate, tautomer, or stereoisomer thereof:

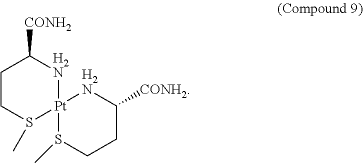

In certain embodiments, the compound comprises the following structure or is a pharmaceutically acceptable salt, N-oxide, solvate, tautomer, or stereoisomer thereof:

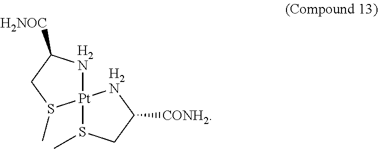

In certain embodiments, processes for making the compounds hereof are provided. For example, compounds 6-11 can be prepared as set forth in Example 1 and/or compounds 6, 9, 12 and 13 can be prepared as set forth in Example 10. Likewise, other compounds hereof can be prepared in accordance with the processes of Examples 1 and/or 10 and such processes otherwise known in the art.

In certain embodiments, the compound is formulated as a prodrug. The term “prodrug” means a derivative of a compound that can hydrolyze, oxidize, or otherwise react under biological conditions (in vitro or in vivo) to provide an active compound, particularly a Pt(II)-thioether complex disclosed herein. Examples of prodrugs include, but are not limited to, derivatives and metabolites of a compound hereof that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, at least one ligand of the compound is substituted with carboxamide or an asteramide.

Specific prodrugs of compounds with carboxyl functional groups are the lower alkyl esters of the carboxylic acid. Carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Formulation of the compound as a prodrug can incorporate hydrolyzable groups which can deactivate the compound in vivo. This can be beneficial in mitigating dose-limiting toxicity issues; a compound can be developed that is effective (i.e., can be dosed at a concentration sufficient to achieve a therapeutic effect) but does not present a significant risk of toxicity (e.g., are low risk for acute kidney injury (AKI)).

The compounds (or pharmaceutically acceptable salts thereof) can contain one or more chiral centers or may otherwise exist as multiple stereoisomers, such as enantiomers, diastereomers, topoisomers, isomers, and enantiomerically or diastereomerically enriched mixtures. Unless stated otherwise, it is intended that all stereoisomeric forms of the compounds are contemplated and structures shown in a particular configuration (e.g., cis or trans) are not limited thereto unless expressly so stated, but instead should be read to encompass all stereoisomers thereof. In certain embodiments, a compound hereof comprises a cis configuration. In certain embodiments, a compound hereof comprises a trans configuration.

“Isomer” refers to structural, geometric, and stereoisomers. The term “geometric isomer” refers to E or Z geometric isomers (e.g., cis or trans) of an alkene double bond. When the compounds contain alkene double bonds, and unless specified otherwise, it is intended that this includes both E and Z geometric isomers (e.g., cis or trans) and/or optical isomers. Likewise, all possible isomers, as well as their racemic and optically pure forms, and all tautomeric forms are also intended to be included to be included.

“Tautomer” means compounds which are capable of existing in a state of equilibrium between two isomeric forms. Such compounds can differ in the bond connecting two atoms or groups and the position of these atoms or groups in the compound.

One of ordinary skill in the art will further appreciate that the compounds can be “deuterated,” meaning one or more hydrogen atoms can be replaced with deuterium. As deuterium and hydrogen have nearly the same physical properties, deuterium substitution is the smallest structural change that can be made. Deuteration is well known to those of ordinary skill in the art.

The compounds (or pharmaceutically acceptable salts thereof) can exist in un-solvated forms as well as solvated forms, including hydrated forms. In general, the solvated forms are equivalent to un-solvated forms. The compounds can exist in multiple crystalline or amorphous forms. In general, all physical forms are equivalent for the uses contemplated. The formulae include pharmaceutically acceptable salts (e.g., acid addition and base salts), hydrates, and/or solvates.

Further, in each of the foregoing and following embodiments, it is to be understood that the formulae include and represent not only all pharmaceutically acceptable salts of the conjugates, but also include any and all hydrates and/or solvates of the conjugate formulae or salts thereof. Indeed, hydrates, solvates, and N-oxides of the conjugates are also contemplated. The term “solvate” means a conjugate, or a salt thereof, that further includes a stoichiometric or non-stoichiometric amount of solvent bound by non-covalent intermolecular forces. Where the solvent is water, the solvate is a hydrate.

It will be appreciated that certain functional groups, such as the hydroxy, amino, and like groups, form complexes and/or coordination compounds with water and/or various solvents, in the various physical forms of the conjugates. Accordingly, the above formulae are to be understood to include and represent those various hydrates and/or solvates.

In each of the foregoing and following embodiments, it is also to be understood that the formulae include and represent any and all crystalline forms, partially crystalline forms, and non-crystalline and/or amorphous forms of the conjugates.

### Pharmaceutically Acceptable Salts

“Pharmaceutically acceptable salts” of the compounds are contemplated. The term “pharmaceutically acceptable salt” refers to those salts whose counter ions can be used in pharmaceuticals. In various embodiments, such salts include, but are not limited to 1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or 2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like. Pharmaceutically acceptable salts are well-known to those skilled in the art, and any such pharmaceutically acceptable salt is contemplated in connection with the embodiments described herein.

In various embodiments, suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

In various embodiments, suitable base salts are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases also can be formed, for example, hemisulphate and hemicalcium salts.

### Pharmaceutical Compositions, Routes of Administration, and Dosing

Further provided is a pharmaceutical composition comprising a compound described herein, a pharmaceutically acceptable salt, N-oxide, solvate, tautomer, or stereoisomer of a compound described herein, and a pharmaceutically acceptable carrier or excipient. The term “pharmaceutically acceptable carrier” means one or more compatible solid or liquid fillers, diluents or encapsulating substances which are suitable for administration to a human or other vertebrate animal. The term “carrier” denotes an organic or inorganic ingredient, natural or synthetic, with which the active ingredient is combined to facilitate the application. The carrier can be an excipient. The choice of carrier can depend on factors such as the particular mode of administration, the effect of the carrier on solubility and stability, and the nature of the dosage form. Pharmaceutical compositions suitable for the delivery of compounds as described herein and methods for their preparation may be found, for example, in Remington: The Science & Practice of Pharmacy, 21st edition (Lippincott Williams & Wilkins, 2005).

The pharmaceutical composition can further comprise a pharmaceutically acceptable excipient.

The components of the compositions also can be commingled with the compound, and with each other, in a manner such that there is no interaction which would substantially impair the desired pharmaceutical efficiency.

The composition can comprise cremophor, polysorbate, nanoparticles, a polymer, or a hydrogel, for example. In certain embodiments, the pharmaceutical composition comprises a plurality of compounds and a pharmaceutically acceptable carrier. In certain embodiments, a pharmaceutical composition further comprises at least one additional pharmaceutically active agent. The at least one additional pharmaceutically active agent can be an agent useful in the treatment of cyanide poisoning.

Pharmaceutical compositions can be prepared by combining one or more compounds with a pharmaceutically acceptable carrier and, optionally, one or more additional ingredients (e.g., pharmaceutically active ingredients). The formulations can be administered in pharmaceutically acceptable solutions, which can routinely contain pharmaceutically acceptable concentrations of salt, buffering agents, preservatives, compatible carriers, adjuvants, and optionally other therapeutic ingredients.

A pharmaceutically acceptable carrier can include any and all solvents, dispersion media, coatings, antibacterial and antifungal agents, isotonic and absorption delaying agents, and the like, and combinations thereof, that are physiologically compatible. The carrier can be suitable for parenteral administration. Pharmaceutically acceptable carriers include sterile aqueous solutions or dispersions and sterile powders for the extemporaneous preparation of sterile injectable solutions or dispersions. Examples of such carriers (or excipients) include, but are not limited to, calcium carbonate, calcium phosphate, various sugars, starches, cellulose derivatives, gelatin, and polymers such as polyethylene glycols. One or more other active agents also can be incorporated into a pharmaceutical composition.

The pharmaceutical composition can be formulated as a liquid, e.g., a suspension or a solution. A liquid formulation can comprise water, ethanol, PEG, propylene glycol, methylcellulose, or a suitable oil, and one or more emulsifying agents and/or suspending agents. A liquid formulation can be prepared by the reconstitution of a solid. In certain embodiments, the composition is suitable (i.e., comprises a formulation suitable for) intramuscular injection.

Pharmaceutical formulations (e.g., for IM administration) include aqueous solutions of the active compounds in water-soluble form. Additionally, suspensions of the active compounds can be prepared as appropriate oily injection suspensions. An aqueous suspension can contain a compound, alone or in further combination with one or more other active agents, in admixture with an appropriate excipient. Excipients include suspending agents, such as sodium carboxymethylcellulose, methylcellulose, hydroxypropyl methylcellulose, sodium alginate, polyvinylpyrrolidone, gum tragacanth and gum acacia; dispersing or wetting agents, such as a naturally-occurring phosphatide, e.g., lecithin; a condensation product of an alkylene oxide with a fatty acid, e.g., polyoxyethylene stearate; a condensation product of ethylene oxide with a long-chain aliphatic alcohol, e.g., heptadecaethyleneoxcycetanol; a condensation product of ethylene oxide with a partial ester derived from fatty acids and a hexitol, such as polyoxyethylene sorbitol monooleate; or a condensation product of ethylene oxide with a partial ester derived from fatty acids and hexitol anhydrides, e.g., polyoxyethylene sorbitan monooleate. The aqueous suspension also can contain one or more preservatives, e.g., ascorbic acid or ethyl, n-propyl, or p-hydroxybenzoate, and one or more coloring agents. In certain embodiments, an aqueous suspension can further comprise suitable lipophilic solvents or vehicles including fatty oils such as sesame oil, or synthetic fatty acid esters, such as ethyl oleate or triglycerides, or liposomes. Optionally, the suspension can also contain suitable stabilizers or agents that increase the solubility of the compounds to allow for the preparation of highly concentrated solutions.

Alternatively, the pharmaceutical compositions can be in powder form for constitution with a suitable vehicle, e.g., sterile pyrogen-free water, before use. Dispersible powders and granules suitable for preparation of an aqueous suspension by the addition of water can provide the active ingredient in admixture with a suspending agent, a dispersing or wetting agent, and one or more preservatives. Additional excipients, for example, coloring agents, also can be present.

Suitable emulsifying agents include naturally occurring gums, e.g., gum acacia or gum tragacanth; naturally occurring phosphatides, e.g., soybean lecithin; and esters, including partial esters derived from fatty acids and hexitol anhydrides, e.g., sorbitan mono-oleate, and condensation products of partial esters with ethylene oxide, e.g., polyoxyethylene sorbitan monooleate. Isotonic agents, e.g., sugars, polyalcohols, such as mannitol, sorbitol, or sodium chloride, can be included in the composition. Prolonged absorption of injectable compositions can be achieved by including in the composition one or more agents to delay absorption, e.g., monostearate salts and gelatin.

For use in therapy or treatment, an effective amount of the compound or composition can be administered to a subject by any mode that delivers the compound as desired. Administering a composition can be accomplished by any means known to the skilled artisan. Routes of administration include, but are not limited to, intravenous, intramuscular, intraperitoneal, subcutaneous, direct injection, inhalation, and topical.

In certain embodiments, a compound can be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, intranasal, and subcutaneous. Suitable means for parenteral administration include needle (including microneedle) injectors, needle-free injectors, and infusion techniques. Where it is desirable to deliver the compound(s) and/or compositions systemically, the compound(s) and/or composition can be formulated for parenteral administration by injection, e.g., by bolus injection or continuous infusion. In certain embodiments, the compound and/or composition hereof can be formulated for intramuscular injection by a single bolus injection.

Formulations for injection can be presented in unit dosage form, e.g., in ampoules or in multi-dose containers, with an added preservative. “Dose” and “dosage” are used interchangeably herein. The compositions can take such forms as suspensions, solutions or emulsions in oily or aqueous vehicles, and can contain formulatory agents such as suspending, stabilizing and/or dispersing agents.

Parenteral formulations are typically aqueous solutions that can contain carriers or excipients, such as salts, carbohydrates, and buffering agents (preferably at a pH of 3-9), but, for some applications, they may be more suitably formulated as a sterile non-aqueous solution or as a dried form to be used in conjunction with a suitable vehicle, such as sterile, pyrogen-free water.

A liquid formulation can be adapted for parenteral administration of a compound. The preparation of parenteral formulations under sterile conditions, for example, by lyophilization under sterile conditions, can readily be accomplished using standard pharmaceutical techniques well-known to those skilled in the art. The solubility of a compound can be increased by the use of appropriate formulation techniques, such as the incorporation of solubility-enhancing agents.

Formulations for parenteral administration can be formulated for immediate and/or modified release. A compound can be administered in a time-release formulation, for example in a composition which includes a slow-release polymer. The compound can be prepared with a carrier that will protect it against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art.

Sterile injectable solutions can be prepared by incorporating the compound(s), alone or in further combination with one or more other active agents, in the required amount in an appropriate solvent with one or a combination of ingredients described above, as required, followed by filtered sterilization. Typically, dispersions are prepared by incorporating the compound into a sterile vehicle, which contains a dispersion medium and any additional ingredients of those described above. In the case of sterile powders for the preparation of sterile injectable solutions, the preferred methods of preparation are vacuum-drying and freeze-drying, which yield a powder of the active ingredients plus any additional desired ingredient from a previously sterile-filtered solution thereof, or the ingredients can be sterile-filtered together.

The pharmaceutical composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating, such as lecithin, by the maintenance of the required particle size in the case of dispersion, and by the use of surfactants.

A compound, or a pharmaceutical composition comprising a compound, can be continuously administered, where appropriate.

### Methods of Treatment, Combination Therapies, and Uses

A use of any of the compounds, pharmaceutical salt, N-oxide, solvate, tautomer, or stereoisomer thereof, and/or pharmaceutical compositions provided herein in the manufacture of a medicament for the treatment of a disease or condition in a subject is provided. The disease or condition can be cyanide poisoning or cyanide exposure.

The medicament can be formulated for intramuscular administration (e.g., intramuscular injection). In certain embodiments, the medicament can be formulated in a single-bolus dosage.

The medicament can be formulated at about or above a pH of 5 (such as at a pH of about 5, 5, 5.5, 6. 6.5, 7, 7.5, 8, 8.5, etc.). The medicament can be formulated at a pH of 7. The medicament can be formulated at a pH of 6.8. The medicament can be formulated at a pH of 5.8.

The medicament can be stored, following formulation for example, at about or below a pH of about 5 (such as at a pH of about 5, 5.0 5.1, 5, 4.9, 4.8, 4.7, 4.6, 4.5, 4.4, 4.3, 4.2, 4.1, 4.0, or at a pH lower than 4.0).

In some embodiments, compounds and pharmaceutically acceptable salts, N-oxides, solvates, tautomers, and stereoisomers described herein (whether part of a medicament, a composition, or the like) contain a positively charged platinum core and from 2 to 6 ligands, at least one of which is a leaving group. That is, the platinum core is amendable to a nucleophilic attack by a cyanide ion, whereby the cyanide ion binds to the platinum core and displaces the leaving group ligand. In some embodiments, each platinum complex is capable of binding from 1 to 6, from 1 to 5, from 1 to 4, or from 1 to 3 cyanide anions. That is, each compound hereof is capable of binding 1, 2, 3, 4, 5, or 6 cyanide anions. In certain embodiments, the compound hereof is capable of binding 4 cyanide anions.

Due to the ability of the platinum core to bind a cyanide anion, the compound, or pharmaceutically acceptable salt, N-oxide, solvate, tautomer, or stereoisomer thereof, can be used as an antidote to cyanide poisoning. In some embodiments, methods of treating (or preventing) cyanide poisoning or cyanide exposure in a subject (e.g., a subject experiencing or at risk of experiencing cyanide poisoning) are provided. In some embodiments, the method comprises administering to the subject a first therapy comprising a therapeutically effective amount of any compound described herein (e.g., Pt-based compound), a pharmaceutically acceptable salt, N-oxide, solvate, tautomer, or stereoisomer thereof, or any of the above-described pharmaceutical compositions. In certain embodiments, at least one ligand of the compound, pharmaceutical salt, N-oxide, solvate, tautomer, or stereoisomer, or pharmaceutical composition comprises an amino acid ligand substituted with one or more carboxamides. The therapeutically effective amount of the first therapy can comprise administering a single dose.

The therapeutically effective amount of the first therapy can be about 3.0-5.5 mg/kg (by weight of the subject). The therapeutically effective amount of the first therapy can be about 3.5 mg/kg (by weight of the subject).

Administering the first therapy can be performed through intraperitoneal (IP) injection of the compound or pharmaceutical composition into the subject. Administering the first therapy can comprise intramuscular injection of the compound, pharmaceutical salt, N-oxide, solvate, tautomer, or stereoisomer thereof, or pharmaceutical composition into the subject.

In certain embodiments, administering comprises intramuscular administration and the compound, pharmaceutically acceptable salt, N-oxide, solvate, tautomer, or stereoisomer thereof, or pharmaceutical composition reaches maximum concentration in the subject (Cₘₐₓ) at about or between 7-9 minutes (such as in 7 minutes to about 9 minutes, in about 7 minutes to 9 minutes, or in 7-9 minutes). As used herein, “maximum concentration” or “Cₘₐₓ” is a pharmacokinetic parameter known to those skilled in the art and means the maximum concentration of the compound/active agent (such as a platinum complex hereof) in serum of a subject after administration to the subject of the compound or pharmaceutical composition hereof by intramuscular, intranasal, subcutaneous, intravenous, or other parenteral route.

In certain embodiments, after the compound, pharmaceutically acceptable salt, N-oxide, solvate, tautomer, or stereoisomer thereof, or pharmaceutical composition is administered intramuscularly to the subject, the compound, a pharmaceutically acceptable salt, N-oxide, solvate, tautomer, or stereoisomer thereof, or pharmaceutical composition undergoes pH-induced isomerization resulting in a reduced cyanide scavenging rate as compared to a cyanide scavenging rate of the compound or pharmaceutical composition within 1 hour of administration to the subject.

The method can further comprise administering to the subject a second therapy. In certain embodiments, the second therapy can comprise administering to the subject a therapeutically effective amount of one or more of glyoxylate, hydroxocobalamin, methemoglobin, riboflavin, methotrexate, sulfanegen, 4-dimethylaminophenol (4-DMAP), dicobalt edetate, glucose, activated charcoal, and cobalt disodium ethylenediaminetetraacetic acid (EDTA); an isotonic fluid intravenously; and/or oxygen therapy (e.g., the provision of supplemental oxygen to the subject).

The second therapy can comprise an agent for ameliorating cyanide-induced oxidative stress within the subject. The agent for ameliorating cyanide-induced oxidative stress can be glyoxylate, for example. In certain embodiments, the second therapy is glyoxylate and the therapeutically effective amount of the first therapy is 3.5 mg/kg (by weight of the subject).

In certain embodiments, the first and second therapies are administered sequentially and/or alternatively relative to each other. In certain embodiments, the first and second therapies are administered concurrently.

Because cyanide inhibits mitochondrial enzyme cytochrome c oxidase, cells of the organism poisoned with cyanide are unable to use oxygen to create ATP, the condition also known as cellular hypoxia. In some embodiments, the methods include identifying a subject who has been exposed to cyanide. Subjects who are in need of treatment using a method or compound described herein can be identified by those of skill in the art, using known methods. Early symptoms of cyanide poisoning can include headache, dizziness, fast heart rate, shortness of breath, and vomiting. These initial symptoms can be followed by seizures, slow heart rate, low blood pressure, loss of consciousness, cardiac arrest, and death. In case of a survival, consequences of cyanide poisoning can be long-term and can include chronic respiratory illnesses (e.g., chronic obstructive pulmonary disease (COPD), asthma, or pulmonary hypertension), blindness, damage and loss of function of vital organs (e.g., heart, lungs, kidneys, and brain), cognitive deficit, and cardiac, neurological, and metabolic dysfunction. In some embodiments, administration of the Pt-based compounds described herein protects from or induces rapid reversal of cyanide-induced pathophysiologic changes.

In one example, cyanide-caused metabolic dysfunction includes disrupted metabolism (e.g., decreased production) of bile acids and purine nucleobases, nucleosides and nucleotides. Some examples of bile acids include glycochenodeoxycholic acid, taurocholic acid, and taurochenodeoxycholic acid. Examples of purine nucleosides and nucleotides include inosine, deoxyadenosine, deoxyguanosine, adenosine, guanosine. Examples of purine nucleobases include purine, adenine, guanine, hypoxanthine, xanthine, theobromine, and uric acid. In other examples, cyanide-caused metabolic dysfunction includes increased concentrations of tricarboxylic acid (TCA) cycle metabolites as the cyanide causes their consumption to slow down. Examples of TCA cycle metabolites include α-ketoglutaric acid, succinic acid, fumaric acid, and malic acid.

In some embodiments, the cyanide poisoning in a subject can be caused by breathing smoke from a fire, exposure to insecticides, administration of medication nitroprusside, an industrial accident, or exposure to chemical warfare. Cyanide is readily absorbed through dermal, bronchial, and digestive routes, rapidly distributes to tissues throughout the body, and can cause multi-organ toxicity, especially to organs having high demand for ATP such as brain and heart. Exposure to milligram amounts of the poison can induce symptoms that appear within minutes of exposure. In some embodiments, any of the methods of use or treatment, or combination therapies mentioned hereinto not include the use of cisplatin or carboplatin.

In some embodiments, the antidote activity (EC₁₀₀) of the compounds is from about 5 μM to about 150 μM, from about 7 to about 100 μM, or from about 10 to about 150 μM.

The methods hereof can reduce, even substantially reduce, systemic and off-target toxicity. “Off-target toxicity” means organ or tissue damage that is not desirable to the physician or other individual treating the subject, or any other effect on the subject that is a potential adverse indicator to the treating physician (e.g., AKI).

In some embodiments, the compounds hereof can be used in cyanide sensing (e.g., in a sensor for cyanide anions present within a subject). When a compound hereof binds cyanide, it turns into a different chemical compound. Thus, the difference is physical properties between the parent compound and the CN adduct (e.g., detectable in UV-vis or IR absorption) can be measured and used to determine the presence of cyanide in a subject.

Combination therapies for treating cyanide poisoning or exposure in a subject are also provided. The combination therapy can comprise administering to the subject: (a) a therapeutically effective amount of a cyanide chelating agent; and (b) a therapeutically effective amount of an agent for ameliorating cyanide-induced oxidative stress within the subject.

The cyanide chelating agent can comprise a platinum(II) thioether comprising bidentate ligands. The cyanide chelating agent can be any compound described herein, a pharmaceutically acceptable salt, N-oxide, solvate, tautomer, or stereoisomer thereof, any of the herein described pharmaceutical compositions, or a compound or pharmaceutically acceptable salt, N-oxide, solvate tautomer, or stereoisomer having the following structure:

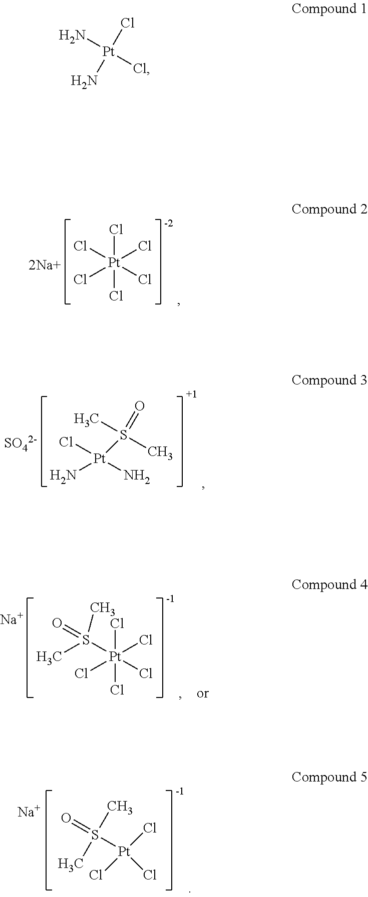

The agent for ameliorating cyanide-induced oxidative stress can be glyoxylate or an analog or functional fragment thereof, for example (and to the extent such functional fragment or analog is capable of ameliorating cyanide-induced oxidative stress in the subject when administered). An “analog” in this context means a compound that is structurally similar to another compound but has slight differences in its structure or properties (e.g., modifications or substitutions in certain parts of the molecule that are not present in the other compound). A “functional fragment” in this context means a chemical structural unit or component of a larger molecule that retains a distinct function or activity of the functions and/or reactivity of the larger molecule. A functional fragment can be linear, branched, or cyclic, an oligomer, or a low molecular weight organic molecule. The functional fragment is fully included within the composition of the original larger molecule.

In certain embodiments, the agent for ameliorating cyanide-induced oxidative stress in the subject comprises a ketone or aldehyde compound selected from the group consisting of glyoxylate, glyceraldehyde, acetaldehyde, dihydroxyacetone, pyruvate, methylglyoxal, and alpha-ketoglutarate.

In certain embodiments, the second therapy is glyoxylate. In certain embodiments, the second therapy is glyoxylate and the therapeutically effective amount of the first therapy is 3.5 mg/kg (by weight of the subject).

The terms “treat,” “treating,” “treated,” and “treatment” (with respect to a disease or condition, such as cyanide poisoning) are used to describe a method for obtaining beneficial or desired results, such as clinical results, which can include, but are not limited to, one or more of improving a condition associated with a disease or condition, curing a disease or condition, lessening severity of a disease or condition, increasing the quality of life of one suffering from a disease or condition, prolonging survival and/or a prophylactic treatment. In reference to cyanide poisoning, in particular, the terms “treat,” “treating,” “treated,” or “treatment” can additionally mean reducing an oxygen concentration within blood of the subject, reduction in cyanide concentration within blood of the subject, stabilizing the subject, preventing progression of the cyanide poisoning, or any other effect on the subject that would be considered by a physician to be a therapeutic or prophylactic treatment of the cyanide poisoning. More particularly, curative treatment refers to any of the alleviation, amelioration and/or elimination, reduction and/or stabilization (e.g., failure to progress to more advanced stages) of a sign/symptom, as well as delay in progression of a sign/symptom of a particular disorder or condition. Prophylactic treatment refers to any of the following: halting the onset, reducing the risk of development, reducing the incidence, delaying the onset, reducing the development, and increasing the time to onset of symptoms of a particular disorder or condition. Desirable effects of treatment can include, but are not limited to, preventing occurrence of the disease or condition, alleviation of symptoms, diminishment of any direct or indirect pathological consequences of the disease or condition, decreasing the rate of disease or condition progression, amelioration or palliation of the disease state or condition, and improved prognosis. In some embodiments, the compounds and compositions hereof can be used to delay development of cyanide poisoning, or to slow (or even halt) the progression of cyanide poisoning.

The term “patient” or “subject” includes human and non-human animals, such as companion animals (dogs and cats and the like) and livestock animals. Livestock animals are animals raised for food production. The subject to be treated is preferably a mammal, in particular a human being.

As used herein, the term “administering” includes all means of introducing the compounds and pharmaceutical compositions comprising same, to the patient. Examples include, but are not limited to, parenteral, systemic/intravenous (iv), intramuscular (im), subcutaneous (sc), transdermal, intrasternal, intraarterial, intraperitoneal, epidural, intraurethral, intranasal, buccal, ocular, sublingual, vaginal, rectal, and the like. In certain embodiments, the route of administration is intramuscular.

Illustrative means of parenteral administration include needle (including microneedle) injectors, needle-free injectors and infusion techniques, as well as any other means of parenteral administration recognized in the art. Parenteral formulations are typically aqueous solutions, which may contain excipients, such as salts, carbohydrates and buffering agents (preferably at a pH in the range from about 3 to about 9). The preparation of parenteral formulations under sterile conditions may readily be accomplished using standard pharmaceutical techniques well-known to those skilled in the art.

The compounds can be formulated as pharmaceutical compositions and administered to a mammalian host, such as a human patient, in a variety of forms adapted to the chosen route of administration. For example, the pharmaceutical composition can be formulated for and administered via parenteral, intravenous, intraarterial, intraperitoneal, intrathecal, epidural, intracerebroventricular, intraurethral, intrasternal, intracranial, intramuscular, topical, inhalation and/or subcutaneous routes. Indeed, the compound, or composition comprising the same, can be administered directly into the blood stream, into muscle, or into an internal organ.

The compounds/compositions can be administered via infusion or injection (e.g., using needle (including microneedle) injectors and/or needle-free injectors). Single-dose injection is often beneficial in treatment of cyanide poisoning and the present compounds and compositions provide significant efficacy when administered intramuscularly in a single dose injection. Solutions of the composition can be aqueous, optionally mixed with a nontoxic surfactant and/or can contain carriers or excipients such as salts, carbohydrates and buffering agents (preferably at a pH of from 3 to 9).

The percentage of the compounds and preparations in a pharmaceutical composition can vary and can be between about 1 to about 99% weight of the active ingredient(s) and a binder, excipients, a disintegrating agent, a lubricant, and/or a sweetening agent (as are known in the art). The amount of the compound(s) in such therapeutically useful compositions is such that an effective dosage level will be obtained.

In some embodiments, the compound(s) is/are administered as a composition comprising one or more pharmaceutically acceptable carriers, adjuvants, diluents, excipients, vehicles, or a combination of any of the foregoing.

The term “therapeutically effective amount” as used herein, refers to an amount of compound that elicits the biological or medicinal response in a tissue system, animal or human that is being sought by a researcher, veterinarian, medical doctor or other clinician (e.g., a desired therapeutic effect), which includes alleviation of the symptoms of the disease or condition being treated. In one aspect, the therapeutically effective amount is that which may treat or alleviate the disease or symptoms of the disease at a reasonable benefit/risk ratio applicable to any medical treatment. However, it is to be understood that the total daily usage of the compounds can be decided by the attending physician within the scope of sound medical judgment. In the treatment of cyanide poisoning, a desired therapeutic effect can range from prevention of experiencing the condition, inhibiting the progression of the condition, and ameliorating symptoms experienced during acute cyanide poisoning. Desirably, the administration of a therapeutically effective amount scavenges a substantial amount of cyanide from the subject's blood in a short period of time and desirably to the point of eradication.

The specific therapeutically effective dose level of the compounds hereof for any particular patient will depend upon a variety of factors, including the state/severity of the condition; the specific composition employed; the age, body weight, general health, gender and diet of the patient; the time and route of administration; the duration of the treatment; drugs used in combination or coincidentally with the compound and/or pharmaceutical composition; and like factors well-known to the researcher, veterinarian, medical doctor or other clinician of ordinary skill. By way of example, a dose of a compound hereof may range from about kg 3.0 to 5.5 mg of the patient's weight (such as about 3.0 mg-about 5.5 mg, about 3.0 mg to about 5.5 mg, or about 3.0 mg to about 5.5 mg). Thus, the absolute amount of compound included in a given unit dosage form can vary widely, and depends upon factors such as the age, weight and physical condition of the subject, as well as the method of administration.

Depending upon the route of administration, a wide range of permissible dosages are contemplated herein. The dosages may be single or divided and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

For clinical use, any compound can be administered in an amount equal or equivalent to 0.2-2,000 milligram (mg) of compound per kilogram (kg) of body weight of the subject per day. The compounds can be administered in a dose equal or equivalent to 2-2,000 mg of compound per kg body weight of the subject per day. The compounds can be administered in a dose equal or equivalent to 5-2,000 mg of compound per kg body weight of the subject per day. The compounds can be administered in a dose equal or equivalent to 20-2,000 mg of compound per kg body weight of the subject per day. The compounds can be administered in a dose equal or equivalent to 100-2,000 mg of compound per kg body weight of the subject per day. The compounds can be administered in a dose equal or equivalent to 200-2,000 mg of compound per kg body weight of the subject per day. Where a precursor or prodrug of a compound is to be administered, it is administered in an amount that is equivalent to, i.e., sufficient to deliver, the above-stated amounts of the compound.

The formulations of the compounds or pharmaceutically acceptable salts, N-oxide, solvate, tautomer, or stereoisomer thereof can be administered to human subjects in effective amounts. Typical dose ranges are from about 0.01 microgram/kg to about 5.5 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments can be used to optimize the dose and dosing frequency for any particular compound or pharmaceutically acceptable salt thereof.

The compounds or pharmaceutically acceptable salts, N-oxide, solvate, tautomer, or stereoisomer thereof can be administered at a concentration in the range from about 0.001 microgram/kg to greater than about 500 mg/kg. For example, the concentration can be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed.

The compounds or pharmaceutically acceptable salts, N-oxide, solvate, tautomer, or stereoisomer thereof can be administered at a dosage in the range from about 0.2 milligram/kg/day to greater than about 100 mg/kg/day. For example, the dosage can be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

The compounds can be administered at a dosage in the range from about 0.25 milligram/kg/day to about 25 mg/kg/day. For example, the dosage can be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

The compound, pharmaceutically acceptable salt, N-oxide, solvate, tautomer, or stereoisomer thereof, or precursor thereof can be administered in concentrations that range from 0.01 micromolar to greater than or equal to 500 micromolar. For example, the dose can be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed.

The compound, or pharmaceutically acceptable salt, N-oxide, solvate, tautomer, or stereoisomer thereof, or precursor thereof can be administered at concentrations that range from 0.10 microgram/mL to 500.0 microgram/mL. For example, the concentration can be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL. 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL, 250.0 micro gram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed.

Multiple infusions may be required in order to treat a subject effectively. For example, 2, 3, 4, 5, 6 or more separate infusions may be administered to a patient at intervals of from about 24 hours to about 48 hours, or every 3, 4, 5, 6, or 7 days. Infusions may be administered weekly, biweekly, or monthly. Monthly administrations can be repeated from 2-6 months or longer, such as 9 months to year.

Administered dosages for the compound for treating cyanide poisoning are in accordance with dosages and scheduling regimens practiced by those of skill in the art. Determining an effective amount or dose is well within the capability of those skilled in the art, especially in light of the detailed disclosure provided herein.

### Kits for Treating Cyanide Poisoning or Cyanide Exposure

Kits are also provided for treating cyanide exposure or cyanide poisoning in a subject. The kit can comprise a drug injection device comprising one or more fluid chambers that are each prefilled with a formulation. The formulation can comprise: (a) a cyanide chelating agent (e.g., such as any of the compounds described herein or a pharmaceutically acceptable salt, N-oxide, solvate, tautomer, or stereoisomer of any compound hereof), or a compound or pharmaceutically acceptable salt, N-oxide, solvate, tautomer, or stereoisomer having the following structure:

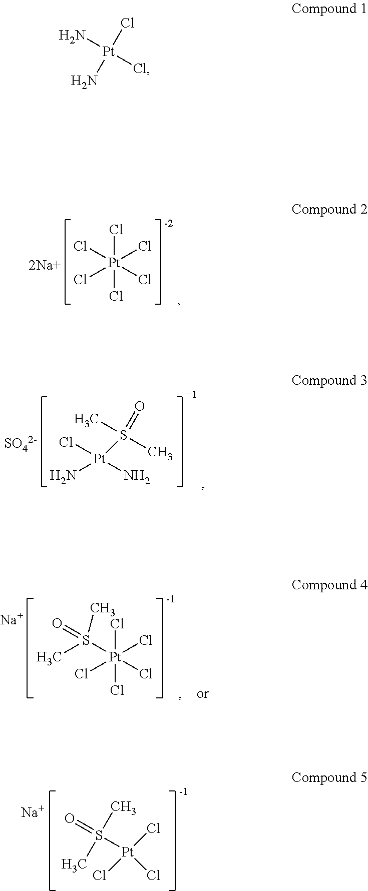

(each an “active agent”), and (b) a pharmaceutically acceptable carrier and/or excipient (“carrier”). The prefilled fluid chamber can be a syringe or a cartridge, for example. In certain embodiments, the active agent and the carrier of the formulation can be separated in the kit (e.g., to facilitate storage). For example, the active agent can be housed in a first fluid chamber and the carrier can be in a second fluid chamber such that the two are mixed as a step prior to administration to the subject. In certain embodiments, the formulation can be stored in a fluid chamber premixed.

The term “cyanide exposure” as used herein means conditions where a subject is either at risk for exposure to cyanide or a derivative thereof or has been exposed to cyanide or a derivative thereof (e.g., via skin contact, eye contact, inhalation, ingestion, or any other mechanism in which the subject comes into contact therewith) but is not yet experiencing detectable symptoms. “Cyanide poisoning” as used herein means and includes cyanide-caused conditions such as cellular hypoxia and related symptoms including headache, dizziness, fast heart rate, shortness of breath, and vomiting. The term “cyanide poisoning” also includes conditions in which a subject is experiencing acute or chronic symptoms of exposure to cyanide or a derivative thereof, irrespective of if those symptoms are readily detectable, such as, and without limitation, cyanide-caused slow heart rate, low blood pressure, loss of consciousness, cardiac arrest, and death. In the case of survival of a poisoned subject, the term also includes cyanide-caused long-term conditions such as chronic respiratory illness, blindness, cognitive deficit, and pathophysiologic changes such as cardiac, neurological, and metabolic dysfunction. The terms cyanide poisoning and cyanide exposure are used interchangeably herein unless expressly stated otherwise.

The drug injection device can be any injection device suitable for administration of the compounds hereof. In certain embodiments, the injection device is configured for injection via one or more needles. The drug injection device can be a hand-held injector. The drug injection device can be an autoinjector. In certain embodiments, the drug injection device is suitable for deployment in the field (e.g., in a mass casualty setting). The drug injection device can be an autoinjector or a hand-held injector.

The formulation can comprise a targeted effective dose of the compound or pharmaceutically acceptable salt, N-oxide, solvate, tautomer, or stereoisomer thereof. For example, each fluid chamber can comprise a single-bolus dose that is immediately ready for administration to a subject and comprises a therapeutically effective amount of the active agent (i.e., compound or pharmaceutically acceptable salt, N-oxide, solvate, tautomer, or stereoisomer thereof). The formulation can comprise a targeted effective dose of the compound or pharmaceutically acceptable salt, N-oxide, solvate, tautomer, or stereoisomer thereof for intramuscular injection.

The formulation can have a pH value of 5 or less. In certain embodiments, the formulation within each fluid chamber can comprise about 1-5 mL (such as about 1 mL to about 5 mL of formulation, about 1 mL to 5 mL of formulation, or 1 mL to about 5 mL of formulation). In certain embodiments, the formulation within each fluid chamber can be within about 3 mL.

Optionally, the kit can comprise instructions, either as inserts or as labels, indicating qualities of the components to be administered, guidelines for administration, and/or guidelines for mixing the components.

The kit can optionally include an additional therapeutic agent (e.g., a therapeutically effective amount thereof) in a second formulation, such as another cyanide antidote or an agent for ameliorating cyanide-induced oxidative stress in the subject, as may be desired or beneficial. In certain embodiments, the second formulation comprises a therapeutically effective amount of an agent for ameliorating cyanide-induced oxidative stress in the subject and a pharmaceutically acceptable carrier and/or excipient. The agent for ameliorating cyanide-induced oxidative stress in the subject can comprise, for example, a ketone or aldehyde compound selected from the group consisting of glyoxylate, glyceraldehyde, acetaldehyde, dihydroxyacetone, pyruvate, methylglyoxal, and alpha-ketoglutarate. The agent for ameliorating cyanide-induced oxidative stress in the subject can comprise glyoxylate or an analog or functional fragment thereof.

### General

All patents, patent application publications, journal articles, textbooks, and other publications mentioned in the specification are indicative of the level of skill of those in the art to which the disclosure pertains.

In the above description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein may be applied to compounds and/or composition components that have configurations other than as specifically described herein. Indeed, it is expressly contemplated that the components of the composition and compounds of the present disclosure may be tailored in furtherance of the desired application thereof.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

The term “about,” when referring to a number or a numerical value or range (including, for example, whole numbers, fractions, and percentages), means that the number or numerical range referred to is an approximation within experimental variability (or within statistical experimental error), and thus the numerical value or range can vary between 1% and 15% of the stated number or numerical range (e.g., +/−5% to 15% of the recited value), provided that one of ordinary skill in the art would consider equivalent to the recited value (e.g., having the same function or result).

When ranges are used herein for physical properties, such as molecular weight, or chemical properties, such as chemical formulae, all combinations and sub-combinations of ranges and specific embodiments therein are intended to be included.

The disclosure may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms. Likewise, the singular forms “a,” “an,” and “the” include plural references unless the context clearly dictates otherwise. Thus, for example, references to “the method” include one or more methods and/or steps of the type, which are described and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

Unless otherwise expressly stated, depicted structures include all stereochemical forms of the structure, i.e., the right-hand (R) and left-hand (S) configurations of each asymmetric center. Therefore, single stereochemical isomers, as well as enantiomeric and diastereomeric mixtures, are within the scope of the present disclosure.

As used herein, the term “composition” generally refers to any product comprising more than one ingredient, e.g., a compound hereof and a carrier.

“Alkyl” generally refers to a straight or branched hydrocarbon chain radical consisting solely of carbon and hydrogen atoms, such as having from one to fifteen carbon atoms (e.g., C₁-C₁₅ alkyl). Disclosures provided herein of an “alkyl” are intended to include independent recitations of a saturated “alkyl,” unless otherwise stated. An alkyl can comprise one to thirteen carbon atoms (e.g., C₁-C₁₃ alkyl). An alkyl can comprise one to eight carbon atoms (e.g., C₁-C₈ alkyl). An alkyl can comprise one to five carbon atoms (e.g., C₁-C₅ alkyl). An alkyl can comprise one to four carbon atoms (e.g., C₁-C₄ alkyl). An alkyl can comprise one to three carbon atoms (e.g., C₁-C₃ alkyl). An alkyl can comprise one to two carbon atoms (e.g., C₁-C₂ alkyl). An alkyl can comprise one carbon atom (e.g., C₁ alkyl). An alkyl can comprise five to fifteen carbon atoms (e.g., C₅-C₁₅ alkyl). An alkyl can comprise five to eight carbon atoms (e.g., C₅-C₈ alkyl). An alkyl can comprise two to five carbon atoms (e.g., C₂-C₅ alkyl). An alkyl can comprise three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Alkoxy” refers to a radical bonded through an oxygen atom of the formula —O-alkyl, where alkyl is an alkyl chain as defined above.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

“Arylalkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain.

The term “heteroalkyl” refers to an alkyl group as defined above in which one or more skeletal carbon atoms of the alkyl are substituted with a heteroatom (with the appropriate number of substituents or valences—for example, —CH₂— can be replaced with —NH— or —O—). For example, each substituted carbon atom is independently substituted with a heteroatom, such as wherein the carbon is substituted with a nitrogen, oxygen, selenium, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). A heteroalkyl is attached to the rest of the molecule at a carbon atom of the heteroalkyl. A heteroalkyl is attached to the rest of the molecule at a heteroatom of the heteroalkyl. A heteroalkyl is a C₁-C₁₈ heteroalkyl. A heteroalkyl is a C₁-C₁₂ heteroalkyl. A heteroalkyl is a C₁-C₆ heteroalkyl. A heteroalkyl is a C₁-C₄ heteroalkyl. Heteroalkyl can include alkoxy, alkoxyalkyl, alkylamino, alkylaminoalkyl, aminoalkyl, heterocycloalkyl, heterocycloalkyl, and heterocycloalkylalkyl, as defined herein.

The terms “protein,” “polypeptide” and “peptide” refer to compounds comprising amino acids joined via peptide bonds and are used interchangeably.

It is recognized that various modifications are possible within the scope of the disclosure. Thus, although the present disclosure has been specifically disclosed in the context of preferred embodiments and optional features, those skilled in the art may resort to modifications and variations of the concepts disclosed herein. Such modifications and variations are considered to be within the scope of the disclosure as claimed herein.

It is therefore intended that this description and the appended claims will encompass all modifications and changes apparent to those of ordinary skill in the art based on this disclosure. For example, where a method of treatment or therapy comprises administering more than one treatment, compound, or composition to a subject, it will be understood that the order, timing, number, concentration, and volume of the administration is limited only by the medical requirements and limitations of the treatment (i.e., two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

Further, the use of headings and subheadings is for ease of reference, given the length of the document. Description under one heading or subheading (such as a subheading in the Detailed Description) is not intended to be limited to only the subject matter set forth under that particular heading or subheading.

### EXAMPLES

The following examples serve to illustrate the present disclosure. The examples are not intended to limit the scope of the claimed invention in any way.

### MATERIALS and METHODS

General methodologies and materials used in the Examples described herein are provided in this section. Any study-specific modifications to these general materials and methods will be set forth in the particular Example.

Materials and Equipment. Na₂[PtCl₄]·nH₂O (n~3, Premion™, Pt 42.4% min) was purchased from Alfa Aesar (Tewksbury, MA). All other chemicals, solvents, TLC plate (silica-gel) and silica-gels were purchased from MilliporeSigma (Sigma-Aldrich, Burlington, MA) or Ambeed (Arlington Hts, IL) and used as received. Smart Evaporator, an equipment capable of removing DMSO at below 50° C. under atmospheric pressure, was acquired from BioChromato Inc. (Kanagawa, Japan).

X-Ray Fluorescence (XRF) of Platinum. An Epsilon 4 spectrometer (Malvern Panalytical, Malvern, UK) with a silver anode X-ray tube was used for these measurements. X-ray fluorescence spectra were collected from 1 mL aqueous solution samples, containing the platinum compounds of Example 1 above as analytes and manganese chloride as an internal standard, pipetted onto mylar foils (ChemPlex, Palm City, FL), embedded in 32 mm polyethylene sample cups (ChemPlex, Palm City, FL). Each sample was irradiated for 20 minutes. The Epsilon 4 software was used to calculate parts per million of each element from each spectrum. The parts per million (ppm) values for platinum were divided by the ppm value for manganese. This ratio was converted to a milligram per milliliter of platinum by comparison to a standard curve generated using sodium hexachloroplatinate hexahydrate.

A standard K₂PtCl₄ ran was measured at 53% which is within 6% of the target amount, consistent with variability observed in other compounds. The weight per volume platinum concentrations were divided by the weight per volume concentrations of the compounds to grams % platinum for each tested compound.

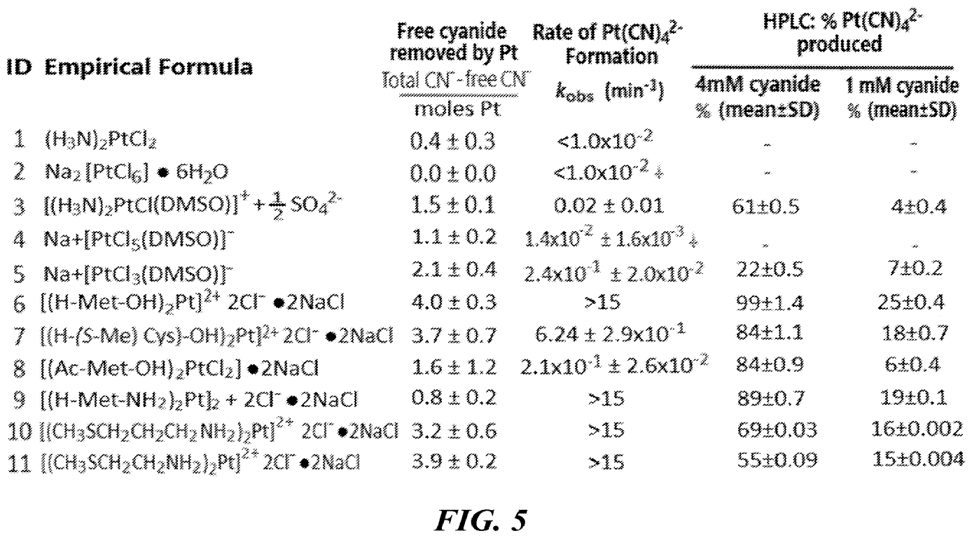

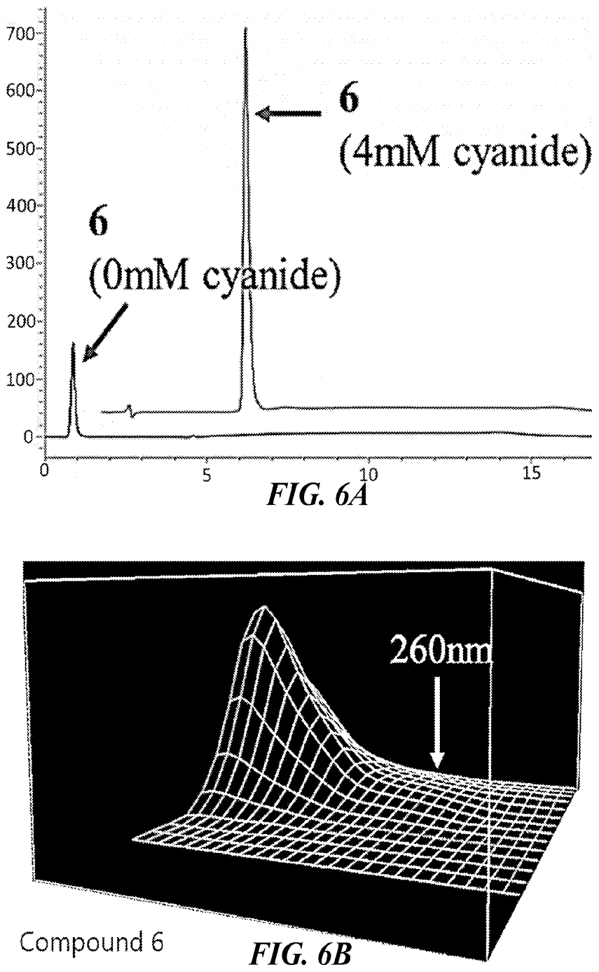

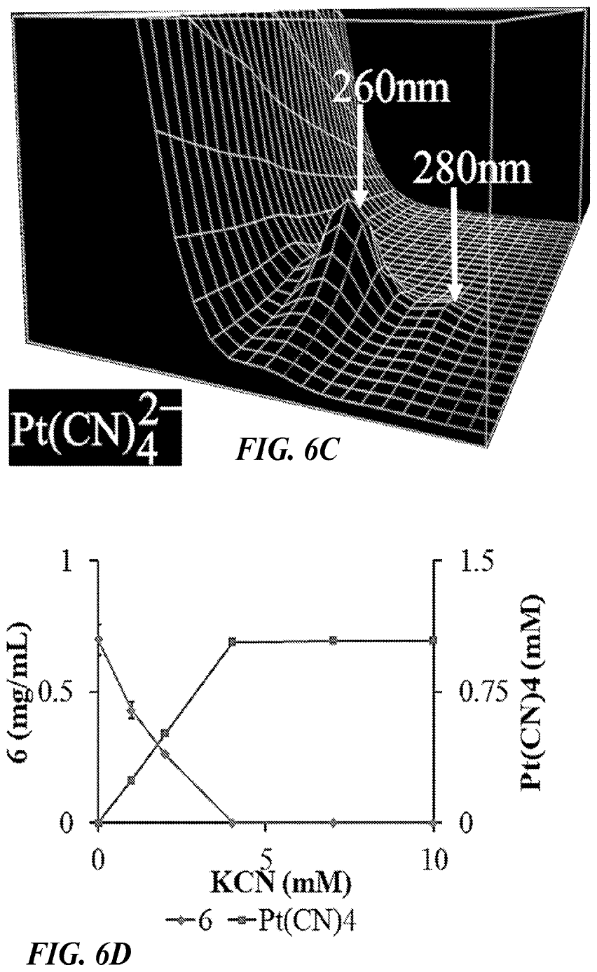

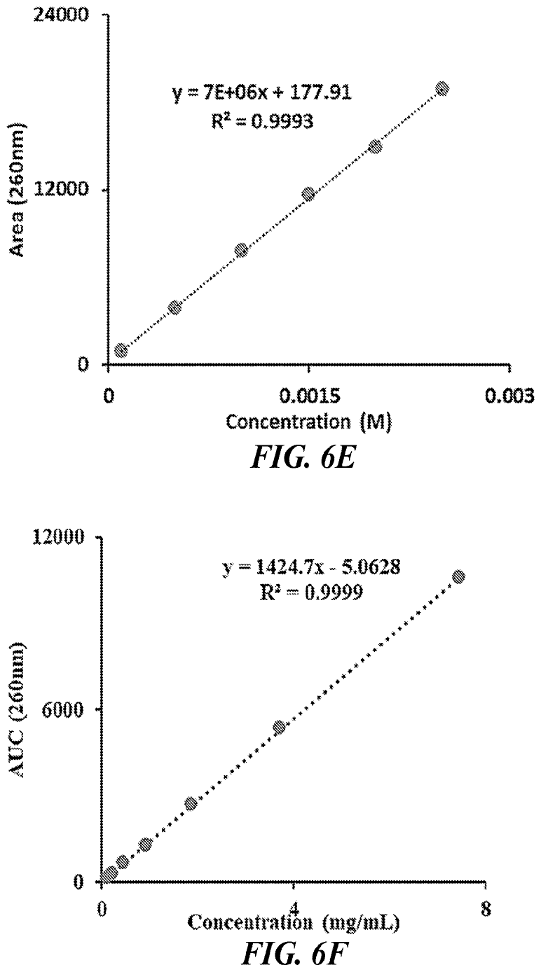

High-Performance Liquid Chromatography (HPLC). HPLC calibration of analytes were performed in aqueous diluent using an Agilent 1100 mode and reverse-phase modes using acetonitrile: water gradients (FIGS. 6E and 6F).

An Agilent 1100 equipped with a Raptor PolarX 50 mm×2.1 mm column was used to separate reaction mixtures of platinum. Stock solutions of each platinum complex of approximately 1 mM were prepared in 0.1M pH 7.5 phosphate buffer. Cyanide was added to an aliquot of the platinum sample at 1, 2, 4, 7, 10 millimolar. Three aliquots of the platinum stock were used for each concentration of cyanide. Analysis of starting material used purified water for mobile phase A, acetonitrile for mobile phase B with a gradient 90% mobile phase B to 50% mobile phase B over 3.5 minutes at 0.3 mL/min. Injection volumes of 5 μL and absorption at 260 nm were used. Analysis of cyanide reaction products used purified water for mobile phase A, acetonitrile for mobile phase B and 10% 200 mM ammonium formate with 0.5% v/v formic acid as mobile phase C with a 0-50% mobile phase B gradient over 5 minutes at 0.3 mL/min. Injection volumes of 5 μL and absorption at 260 nm was used for all analyses.

Normalized Pt(CN)₄²⁻ was calculated as the difference of moles cyanide added (x) and measured Pt(CN)₄²⁻ divided by the range of Pt(CN)₄²⁻ for the titration sample set (see Eq. 1 below).

$$Normalized {Pt ( CN )}_{4}^{2 -} = \frac{x - {Pt ( CN )}_{4}^{2 -}}{{Pt ( CN )}_{4}^{2 -} range} x 100 \qquad ( Eq . 1 )$$

Ion-Selective Electrode for the Detection of Cyanide (ISE). An Orion ion-selective electrode for cyanide was purchased and used from Thermo Fisher Scientific (Waltham, MA). The electrode was calibrated each day of use with freshly prepared cyanide standards 0.26 ppm-26.0 ppm as instructed by the product manual. Every 2 hours the electrode drift was verified to be ≤2.0% as specified by the acceptance criteria in the manual.

Titrations of the cyanide and platinum solution were performed as follows: volume of the platinum mixture containing 0.1 mM platinum and adjusted to pH>10 using NaOH to maintain cyanide in solution. Titrations were then performed by adding small volumes equal to 1% v/v of the initial solution.

Ultraviolet-Visible Spectrophotometry (UV-Vis). A Cary60 (Agilent Technologies, Inc., Santa Clara, CA) was used for all the UV-Vis experiments to monitor the reaction between platinum and cyanide. All cyanide reactions were pH 7.6 using 12.5 mM NaPi with 0.8 mM KCN and approximately 0.02 mM platinum. Data collection was at 24,000 nm/sec between 300-220 nm averaging time intervals of 0.65 seconds for the first 30 seconds and once every 5 minutes. Kinetic data were fit using a one-phase exponential curve to derive apparent rate constants.

UV-Vis kinetics and platinum content: The rates of change for the platinum spectra were modeled assuming first-order kinetics. In Eq. (2), A is the absorbance. The rate of change (k₁) can be obtained as the slope when ln(A_{∞}−Aₜ) plotted against time (t). Tseng et al., Characteristics and applications of the Lagergren's first-order equation for adsorption kinetics, J Taiwan Institute Chem Engineers 41: 661-669 (2010). Each assay was monitored for at least 10 minutes or until the sample appeared to reach equilibrium. By rearranging Eq. (2), the half-life could be assessed using Eq. (3). The observed rate constants and the respective observed half-lives were reported after following the reaction to a minimum of 1 half-life.

$$ln ( {A}_{∞} - {A}_{t} ) = ln ( {A}_{∞} - {A}_{t} ) - {k}_{1} t \qquad ( Eq . 2 )$$

$${t}_{\frac{1}{2}} = \frac{ln ( 2 )}{{k}_{obs}} \qquad ( Eq . 3 )$$

Characterization of Pt Compounds by NMR. Dried powder samples were dissolved an analyzed by ¹⁹⁵Pt NMR to monitor the deshielding effect on the platinum core and observed as shifts in the ≤−2800 ppm with hexachloroplatinate reference.

¹⁹⁵Pt spectra were acquired using a Bruker DRX 500 MHz spectrometer equipped with a BBFO probe operating at room temperature. ¹H and ¹³C spectra were acquired using the same spectrometer or a Bruker Avance 800 MHz spectrometer equipped with a cryo-TCI probe at 25° C. The water-soluble Pt(II) compounds in the range of 20-100 mM in an aqueous buffer containing 10% D₂0 were used for analyses. In the case of low water solubility complexes (MetPt(II)Cl₂) was dissolved at 50 mM in d7-DMF. ¹⁹⁵Pt spectra have a spectral width of 933 ppm with a center at −3600 ppm (or −2800 ppm for MetPt(II)Cl₂ in d7-DMF). Excitation angles were about 50-60 degrees with an acquisition time of 0.16 seconds and recycling delay of 0.7 seconds, and the total number of scans was 2048. Exponential window functions with 50 Hz line-broadening were applied to FIDs prior to Fourier transformation, manual phasing, and automatic baseline corrections.

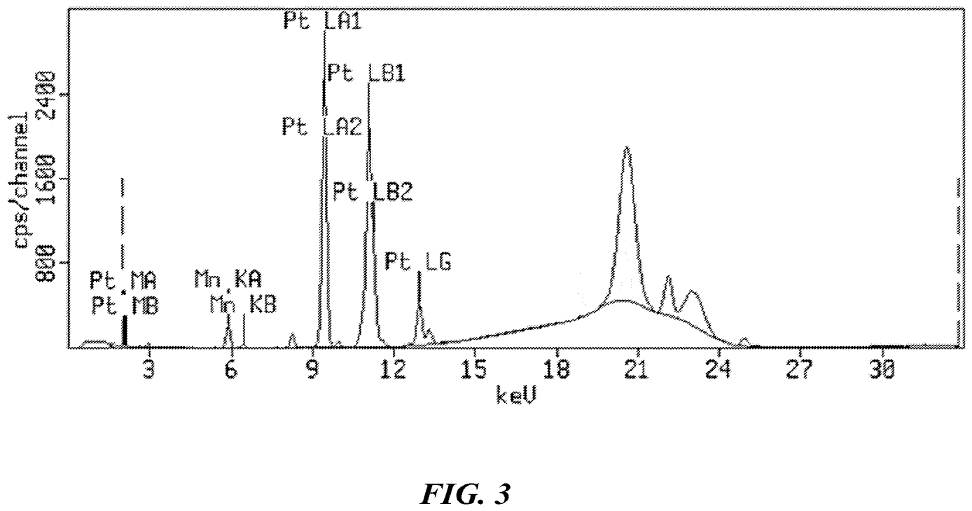

At low pH (pH<7.0), rapid conversion between bicyclic and hemicyclic isomers in the bidentate ligands was observed. Assignments for the ¹H spectra were not made for these compounds due to the complexity. For example, compound 0 shows a mixture of isomers (FIG. 3) in ¹⁹⁵Pt NMR with 4 signals from −3550 to −3700 ppm which was observed as pH dependent. Norman et al. (1992), supra proposed the dominate peaks under acidic conditions (e.g., −3625 ppm and −3675 ppm) as the bidentate ring closed structure. ¹⁹⁵Pt spectra were simplified between pH values of 7 and 8 and were consistent with Norman's observations suggesting a ring-closed dominate form.

Zebrafish Efficacy. Zebrafish embryos 6 days post fertilization were grown in baths containing lethal amounts of cyanide and varying amounts of platinum and ligand ratios (as described in more detail below). Efficacy reported is the minimum concentration of platinum required to achieve 100% survival of zebrafish embryos.

Inductively Coupled Plasma Mass Spectrometry (ICP-MS) Digestion. In 15 mL polypropylene centrifuge tubes, 90 μL of rabbit blood samples were mixed with 800 μL of 30% v/v hydrogen peroxide (Macron Fine Chemicals; Avantor, Radnor Township, PA). Subsequently, 800 μL of Aristar ultrapure hydrochloric acid, and 400 μL Aristar ultrapure nitric acid was added. The addition of reagents to blood samples resulted in vigorous bubbling. The solutions were loosely capped and incubated at room temperature until bubbling ceased. The solutions were then heated in a water bath at 60° C. for 16 hours. Each sample was diluted with MilliQ water to 10 mL, gently centrifuged at 1000×G, for 5 minutes to sediment particulates, and then transferred to new 15 mL polypropylene tubes. These samples were then used for ICP-MS analysis.

Quantification of Platinum by ICP-MS for Pharmacokinetics. A Thermo Fisher Scientific Element II (Waltham, MA) inductively coupled plasma mass spectrometer was used in all ICP-MS analyses. Mass offsets were calculated using a multi-element standard containing Au, Ir, Os, Pd, Pt, Re, Rh, Ru in 10% hydrochloric acid (VWR). Samples were introduced into the mass spectrometer using an Arridus peristaltic pump. Samples were analyzed with take up times of 90 seconds and sample collection times of 90 seconds each. Platinum-195 peak intensities were averaged over the sample collection time. These averaged values were converted to nanograms or micrograms per milliliter by comparison to standard curves generated with sodium hexachloroplatinate standard curve in rabbit serum.

Kinetics. Platinum was reacted with cyanide under first order conditions in platinum. Pt(CN)⁴²⁻ was a product for the compounds described herein. Signal at 255 nm increased over time and was transcribed as Pt(CN)⁴²⁻ formation. Results were plotted as production of Pt(CN)⁴²⁻ over time and the observed rate constants were obtained by fitting the curve to a first order process. Conditions for the reaction were performed in 12.5 mM phosphate buffer, pH 7.6 with 0.8 mM KCN and 0.02 mM platinum.

Mass Spectrometry (High Resolution). Platinum agents were dissolved in 50:50 acetonitrile and water solvent. The sample was infused into a LTQ Orbitrap mass spectrometer (Thermo Fisher Scientific, Waltham, MA) using electrospray ionization in either positive or negative mode.

Mass Spectrometry (Low Resolution). Platinum compound 8 was dissolved in 50:50 acetonitrile and water solvent. The sample was infused into an Advion Expression spectrometer (Advion, Inc., Ithaca, NY) using electrospray ionization in either positive or negative mode.

### Example 1

### Generation of Platinum Compounds

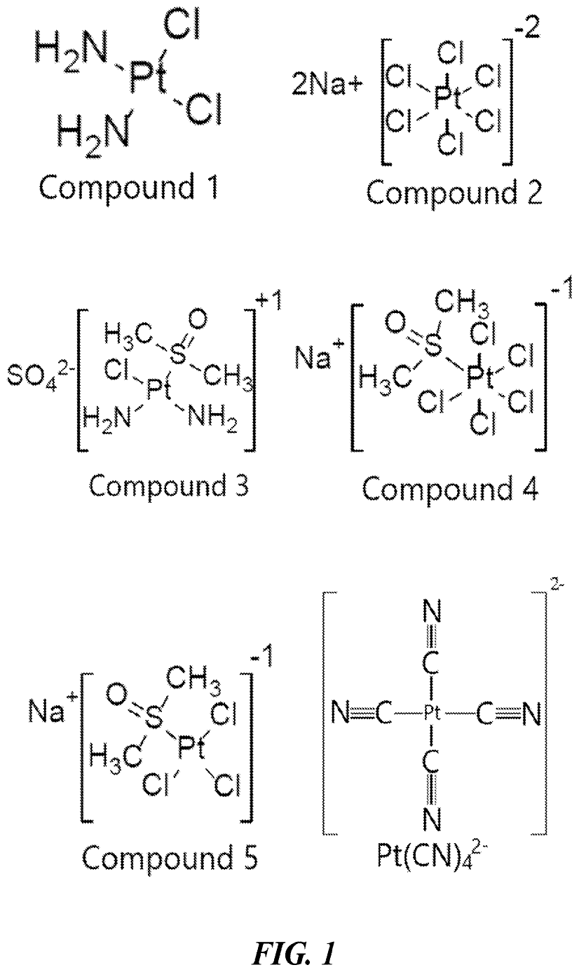

Previously, cisplatin and sodium hexachloroplatinate (HCP, Na₂[PtCl₆]) were found to be active as cyanide antidotes only when formulated with dimethylsulfoxide (DMSO). Based upon the known reactivity, it was contemplated that DMSO served as a ligand on platinum and facilitated rapid substitution for cyanide at the metal. The DMSO complex of cisplatin has reduced toxicity and efficacy as a deoxyribonucleic acid (DNA) damaging agent. A method for preparing a sulfate salt of cisplatin-DMSO (compound 3) from compound 1 was used that made use of silver nitrate to capture released chloride to promote full conversion (FIG. 1).

Compound 3: Briefly, to generate compound 3, a mixture of cisplatin (3120 mg, 10.4 mmol), silver sulfate (1621 mg, 5.20 mmol), deionized water (104 mL), and DMSO (3.74 mL, 52.7 mmol) was placed in a 250 mL round-bottom flask and stirred high-speed in dark at ambient temperature for 5 days. The heterogenous mixture was centrifuged in 50 mL falcon tubes to remove silver chloride and silver-black precipitates. The supernatant was collected and concentrated to 10 mL at 35° C. in vacuo, before transferring to a 15 mL capped plastic tube, and centrifuged at 7500 rpm for 2 minutes. The supernatant was centrifuged again at 14,000 rpm for 3 minutes to completely remove the silver precipitates. To recover any compound 3 from the precipitates, the solids were dissolved in water and clarified by centrifugation. The combined solutions were placed in a 50 mL glass bottle and dried in Smart Evaporator at 35° C. overnight. The resultant solid residue was triturated and washed with ethanol (3 mL×4 times) followed by diethyl ether (3 mL×3 times) and dried in vacuo to afford compound 3 (4.15 g) as a light gray powder.

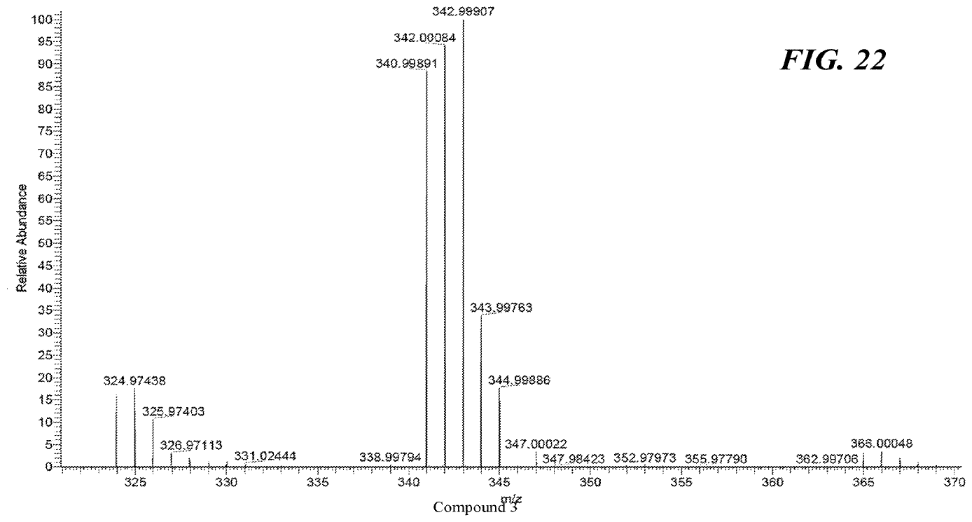

TLC: Rf 0.5 on silica-gel, 1,4-dioxane/water=4/1, UV254 and ninhydrin. ESIMS (positive ion, solvent: water and acetonitrile), calculated for [C₂H₁₂ClN2OPtS]+: 342.000673; found: 342.000084. Mass spectrum data of compound 3 shown in FIG. 22 (using the LTQ Orbitrap in electrospray ionization positive ion mode; solvent used was a 1:1 mixture of acetonitrile and water; m/z of 342.00084 was obtained with an error of −0.48 ppm).

Two additional DMSO compounds 4 and 5 were also prepared from compound 2 and sodium tetrachloroplatinate, respectively, for further chemical and pharmacological evaluation.

Compound 4: The preparation of Pt(IV) compound 4 was in analogy to the original formulation used to prepare the cyanide antidotes. Compound 4 could be isolated from simple column chromatography after exposure to excess DMSO.

Briefly, to generate compound 4, a mixture of HCP (Na²[PtCl⁶]·6H²O, 250 mg, 0.445 mmol), DMSO (252 μL, 3.55 mmol), and deionized water (252 μL) was warmed at 45-50° C. in a 20 mL glass vial for 1 hour. The mixture was diluted with deionized water (4.5 mL) and evaporated using Smart Evaporator at 30° C. for 2.0 hours and the process repeated. The resulting yellow-orange colored residue was purified by column chromatography (silica-gel, 230-400 mesh, 5 g; 2.0 cm×3.5 cm) using a gradient of dichloromethane/acetone=4/1 to 1/1. The recovered yellow-orange, viscous liquid was dried first by blowing argon gas flow to remove residual solvent. The semi-solid was finely divided before final drying at reduced pressure to afford compound 4 (170 mg) as yellow powder.

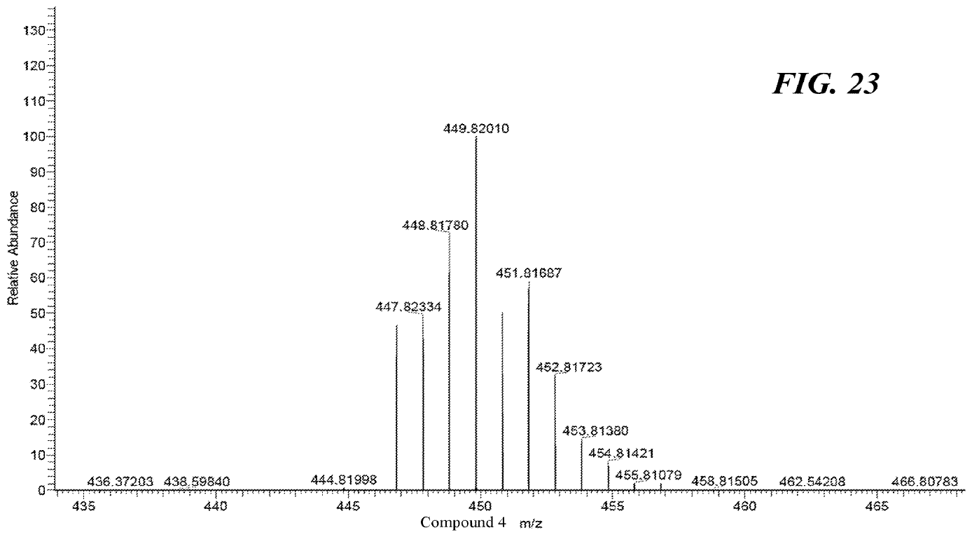

TLC: Rf ~0.4 on silica-gel, dichloromethane/acetone=1/1, UV254 and ninhydrin. High resolution ESI-MS (negative ion, mobile phase: acetone), calculated for [PtCl₅(DMSO)]: 446.8209; found: 446.8196. Mass spectrum data of compound 4 shown in FIG. 23 (using the LTQ Orbitrap in electrospray ionization negative ion mode; solvent used was a 1:1 mixture of acetonitrile and water; m/z of 447.82334 was obtained with an error of −0.79 ppm).

Compound 5: A protocol similar to the one used to generate compound 4 was used to prepare compound 5 from a reaction of DMSO with tetracholoplatinate. To generate compound 5, Na₂[PtCl₄]·nH₂O (n~3) (200 mg, 0.416 mmol) was dissolved in deionized water (910 μL) and treated with DMSO (33 μL, 0.46 mmol). The resulted brownish homogenous solution was kept in dark at ambient temperature overnight. The mixture became a yellowish solution containing needle-like solid over this time period. Solvent was removed using a Smart Evaporator at 40° C. until dry. The yellow solid residue was extracted with 3/1 acetone/methanol (1 mL×3 times) until the insoluble solid became almost white in color. The entire procedure was repeated at 6-times scale and the combined yellow extracts were dried in vacuo. The recovered solid was then treated with 2×2 mL of ethanol and dried in vacuo to remove residual water. Compound 5 (1.08 g) was recovered as a yellow powder.

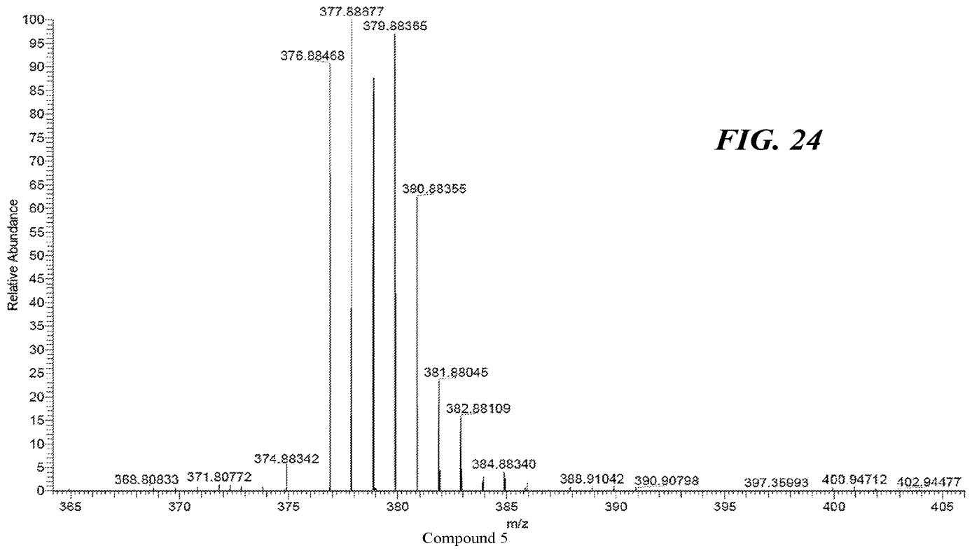

TLC: Rf 0.6 on silica-gel, dichloromethane/acetone=1/3, UV254 and ninhydrin. ESI-MS (negative ion, mobile phase: acetonitrile and water) calculated for [C₂H₆Cl₃OPtS]⁻; 377.885281; found: 377.88677. Mass spectrum data of compound 5 shown in FIG. 24 (using the LTQ Orbitrap in electrospray ionization negative ion mode; solvent used was a 1:1 mixture of acetonitrile and water; m/z of 377.88677 was obtained with an error of 3.94 ppm).

### Example 2

### Ligand Selection of Sulfur Containing Pt(II) Compounds

A preliminary study was designed to guide the criteria for ligand selection of sulfur containing Pt(II) compounds. Naturally occurring sulfhydryl, sulfide, and sulfoxide ligands/metabolites were tested as replacements for DMSO in combination with compound 2 using the zebrafish cyanide toxicity model as set forth in Table 1.

Briefly, zebrafish larvae were plated into 96-well plates at 6 days post fertilization in hydroxyeicosapentaenoic acids (HEPEs) buffered Tubingen E3 medium (n=5 per well). Larva was dosed with potassium cyanide (50 μM) and each compound (i.e., platinum complex) to be tested (1-250 μM) in each well. The cyanide concentration used represented the lethal dose (LD100) for all embryos in each well and survival was documented after 4 hours of exposure post-treatment.

The effectiveness for each of these samples was determined from 3-4 replicates and no rescue (NR) was observed using 1:10 Pt:ligand ratio of L-Cysteine, L-Glutathione, L-Methionine sulfoxide, and L-Alliin.

TABLE 1
Efficacy of hexachloroplatinate when mixed
with stoichiometric amounts of indicated ligand.
EC100 in Zebrafish (μM)

| | |
|---|---|
| S-Ligand containing | Molar Ratio (PtCl₆⁻²:Ligand) |

| ligand used | 10:1 | 1:1 | 1:10 |
|---|---|---|---|
| S-Methyl-L-Cysteine | 125 | 31 | 16 |
| L-Methionine | 125 | 31 | 16 |
| L-Cysteine | 125 | 62 | NR |
| L-Glutathione (reduced) | 250 | 62 | NR |
| L-Methionine sulfoxide | NR | NR | NR |
| (±)-L-Alliin | NR | NR | NR |

Controls
PtCl₆⁻² + DMSO EC100 = 62 μM
Hydroxocobalamin EC100 = 125 μM

A mixture of compound 2 with either L-methionine or S-methyl-L-cysteine in water at 1:1 or 1:10 (Pt to ligand) ratios provided cyanide rescue in zebrafish. The potencies observed were greater than 4-fold improved as compared to those observed in the original DMSO formulations.

In the case of compounds with free sulfhydryl (L-cysteine and L-glutathione reduced), a 1:1 mixture provided enhanced rescue potencies, but this activity was lost when the ligands were in molar excess.

Finally, compound 2 in mixtures with sulfoxide amino acids (L-methionine sulfoxide or L-alliin) did not provide any cyanide rescue activity. Although promising, the potential for compound 2 to oxidize naturally occurring sulfur containing amino acids while complexed is a known process. As a result, sulfide ligands for Pt(II) complexes offered an alternative focus for the design of additional candidate cyanide antidotes.

### Example 3

### Generation of Platinum Compounds with Modifications of the Carboxylate and Amino Groups

In previous efforts, the platinum (II) (Pt(II)) complex (comprising two dimethylsulfide ligands) provided rescue of cyanide toxicity in the zebrafish model described herein in the absence of DMSO. Phosphate buffered saline (PBS) and DMSO solvated compounds were equally as efficacious showing a 2-fold greater potency with respect to cisplatin prepared in DMSO. Nath et al. (2017), supra. Based upon the established chemical hypothesis that the sulfur ligands can direct the reactivity of nucleophile additions to Pt(II), additional compounds were prepared to serve as improved cyanide scavenging agents, including a series of Pt complexes with modifications of the carboxylate and amino groups, including N-acetyl-L-methionine (compound 8), L-methionine amide (compound 9), 3-(methylthio)propylamine (compound 10) and 2-(methylthio)ethylamine (compound 11) were prepared for comparing the structural variation impact on reactions with cyanide.

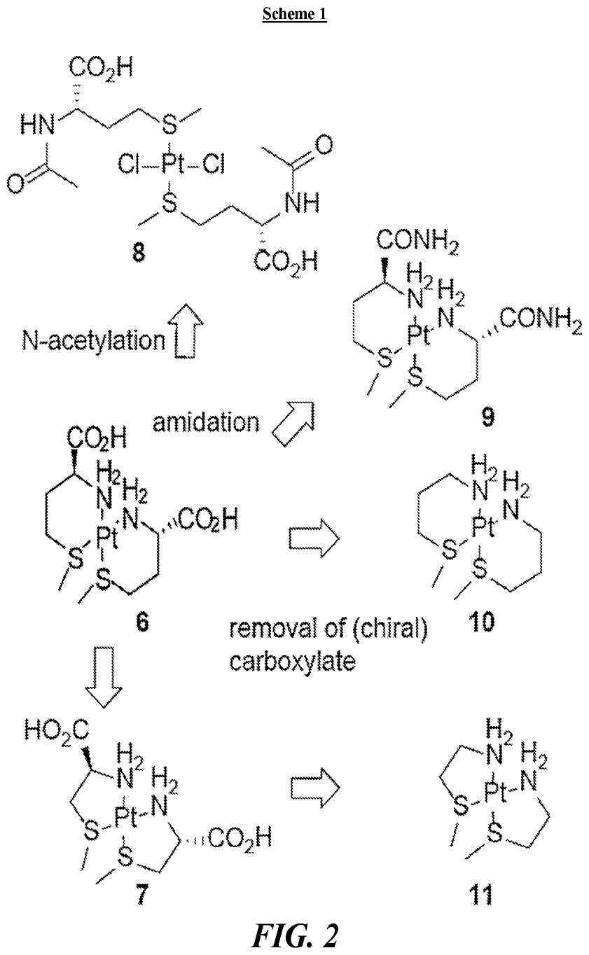

Sulfide ligands to platinum were expected to yield strong interactions. Generally, each of the sulfide complexes compounds 6-11 were prepared in water before isolation as dried powders. No attempts were made to remove the salt products. There are several isomeric species anticipated based upon the ligand structures. Except for compound 8, all the ligands have nitrogen centers capable of bidentate coordination with Pt in square planar configurations. Previous detailed assignments guided the assignments of the major isomeric form of Pt complexes. Using NMR characterization of aqueous solutions, the assignments of the major structures were made as represented in Scheme 1 shown in FIG. 2.

Compound 6: To generate compound 6, a mixture of Na₂[PtCl₄]·nH₂O (2224 mg, 5.09 mmol), L-methionine (1.45 g, 9.72 mmol) and deionized water (10.2 mL) was sonicated for 5 minutes until all solids were dissolved. The homogenous mixture was kept in dark at ambient temperature for 2 hours and then centrifuged to remove platinum black particles generated in situ. The light-yellow supernatant was shell frozen and lyophilized overnight. The incomplete lyophilized mixture was refrozen and lyophilized a second time overnight to isolate compound 6 (3543 mg) as light-yellow powder.

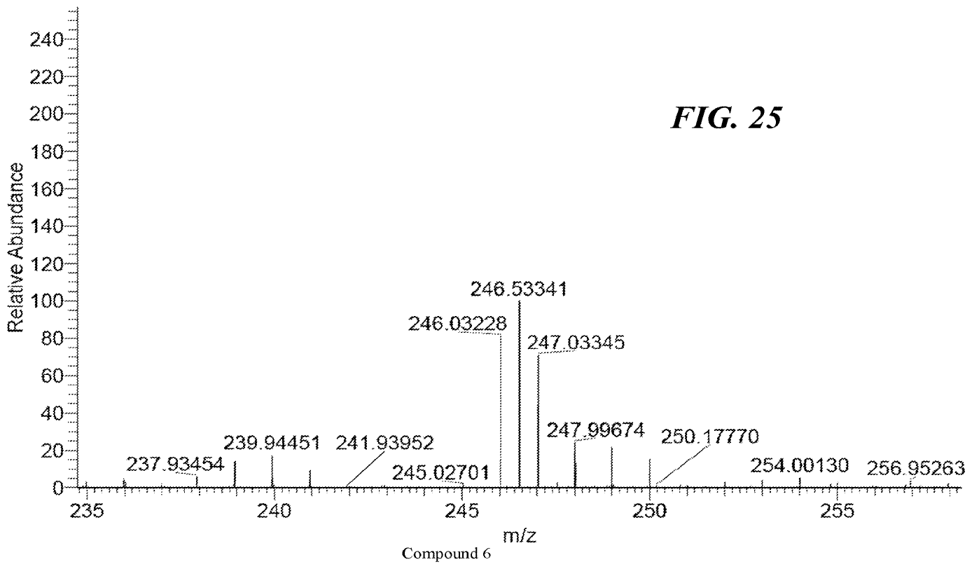

TLC: Rf 0.3 silica-gel, 1% NaCl in water, UV254 and ninhydrin. ESI-MS (positive ion, mobile phase: water and acetonitrile) calculated for [C₁₀H₂₂N₂O₄PtS₂]²⁺: 246.5334435; found: 246.53341. Mass spectrum data of compound 6 shown in FIG. 25 (using the LTQ Orbitrap in electrospray ionization positive ion mode; solvent used was a 1:1 mixture of acetonitrile and water; m/z of 246.53341 was obtained with an error of −0.13 ppm).

Compound 7: To generate compound 7, a mixture of Na₂[PtCl₄]·nH₂O (257 mg, 0.588 mmol), L-(S-methyl)cysteine (160 mg, 1.18 mmol) and deionized water (2.9 mL) was sonicated for 5 minutes or until all solids were dissolved. The homogenous mixture was kept in dark ambient temperature overnight and evaporated at 35° C. using Smart Evaporator. The pale-green sticky residue was extensively triturated using a spatula and sonication in ethanol (5 mL) until the mixture became a uniform suspension. The solid in this mixture was isolated by high-speed centrifuge, washed with diethyl ether (2 mL, ×3 times), and dried to afford compound 7 (295 mg) as a white powder.

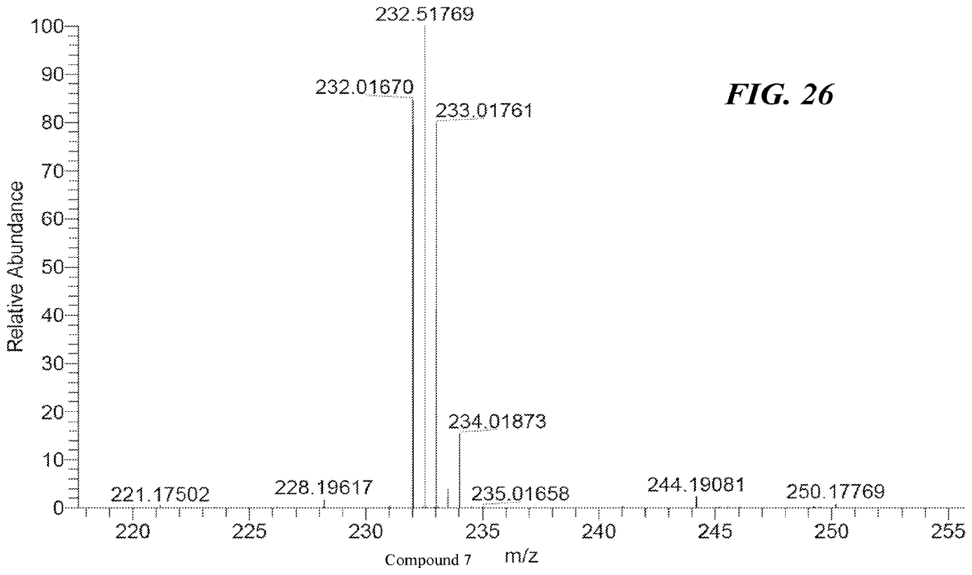

TLC: two spots, Rf 0.3 tailing and 0.6 tailing on silica-gel, 1% NaCl in water, UV254 and ninhydrin. ESI-MS (positive ion, mobile phase: acetonitrile and water), calculated for [C₈H₁₈N₂O₄PtS₂]²⁺: 232.5177935; found: 232.51769. Mass spectrum data of compound 7 shown in FIG. 26 (using the LTQ Orbitrap in electrospray ionization positive ion mode; solvent used was a 1:1 mixture of acetonitrile and water; m/z of 232.51769 was obtained with an error of −0.44 ppm).

Compound 8: To generate compound 8, a mixture of Na₂[PtCl₄]·nH₂O (654 mg, 1.50 mmol), L-(N-acetyl)methionine (580 mg, 3.03 mmol) and deionized water (4.50 mL) was sonicated for 5 minutes until all solids were dissolved. The homogenous mixture was kept in dark at ambient temperature overnight and evaporated at 40° C. to afford light-yellow pasty residue. The residue was dissolved with ethanol (2 mL) and dried in vacuo (repeated twice), affording an amorphous foam residue, which was crushed with a spatula and dried in vacuo to isolate compound 8 as pale yellow-greenish powder (985 mg).

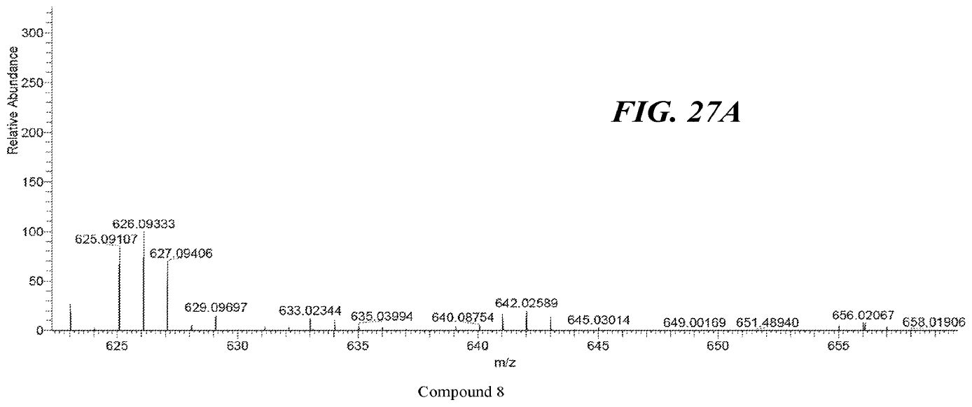

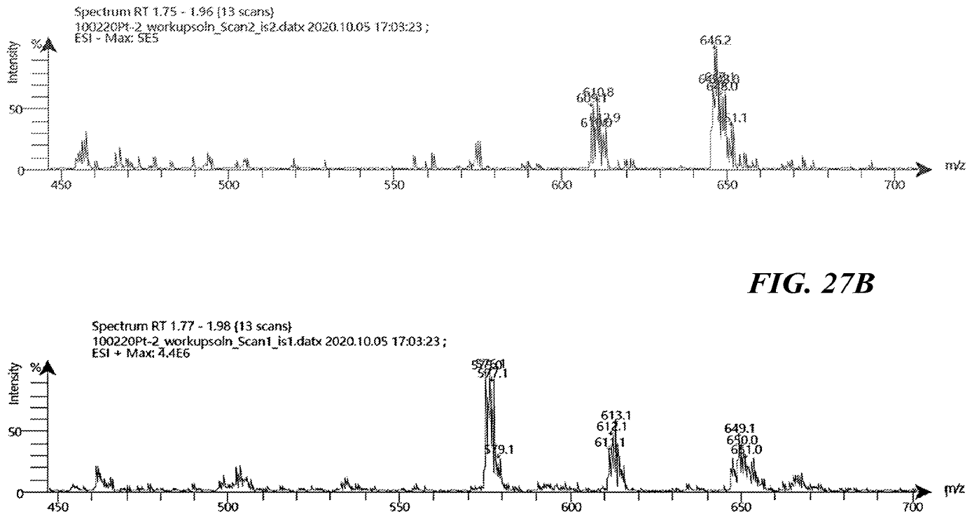

TLC: two spots, Rf ~0.5 and 0.7, 1,4-dioxane/water=9/1, UV254 and ninhydrin. ESI-MS (positive ion, mobile phase: acetonitrile and water), calculated for [C₁₄H₂₆Cl₂N₂O₆PtS₂]: 648.47; found: 646. Mass spectrum data of compound 8 shown in FIGS. 27A and 27B (using the LTQ Orbitrap in electrospray ionization positive ion mode (FIG. 27A; solvent used was a 1:1 mixture of acetonitrile and water; 626 m/z was identified as [Ac-Met₂Pt(HO)(H₂O) Na]+); using an Advion Expression spectrometer in electrospray ionization positive ion (top) and negative ion (bottom) modes (FIG. 27B; solvent used as a 1:1 mixture of acetonitrile and water; 646 m/z was identified as [Ac-Met₂PtCl₂ Na]⁻)).

Compound 9: To generate compound 9, as a preliminary trial, a mixture of Na₂[PtCl₄]·nH₂O (119 mg, 0.272 mmol), L-methionine amide hydrochloride (81 mg, 0.44 mmol) and deionized water (1.36 mL) was briefly sonicated to create a homogenous mixture. After 0.5, the reaction was checked with TLC, which indicated product formation. A second reaction mixture of Na₂[PtCl₄]·nH₂O (150 mg), L-methionine amide hydrochloride (102 mg) and deionized water (1.7 mL) was made in the same manner. Both mixtures were combined and dried using Smart evaporator at 35° C. overnight. The residue was triturated using a spatula and sonication in ethanol (3 mL), washed with diethyl ether (2 mL, ×2), and dried to afford compound 9 (428 mg) as beige-white powder.

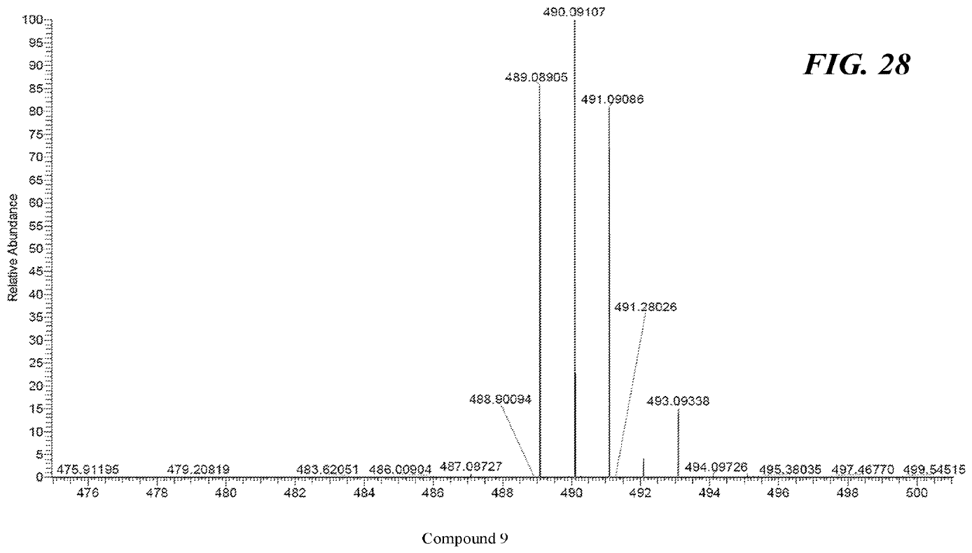

TLC: Rf 0.15 tailing on silica-gel, 1% NaCl in water, UV254 and ninhydrin. ESI-MS (positive ion, mobile phase: acetonitrile and water), calculated for [C₁₀H₂₄N₄O₂PtS₂]²⁺: 490.09103; found: 490.09107. Mass spectrum data of compound 9 is shown in FIG. 28 (using the LTQ Orbitrap in electrospray ionization positive ion mode; solvent used was a 1:1 mixture of acetonitrile and water; m/z of 490.09107 was obtained with an error of −0.08 ppm).

Compound 10: To generate compound 10, Na₂[PtCl₄]·nH₂O (485 mg, 1.11 mmol) was dissolved in deionized water (5.55 mL) and added 3-(methylthio)propylamine (255 μL, 2.28 mol). The heterogeneous mixture was stirred at ambient temperature for 5 hours. The resulting homogenous mixture was evaporated using Smart Evaporator at 35° C. overnight to dryness. The residue was triturated using a spatula and washed with IPA (3 mL×3 times) using a sonicator, followed by diethyl ether (3 mL×2), then dried to afford compound 10 (572 mg) as a beige-white powder.

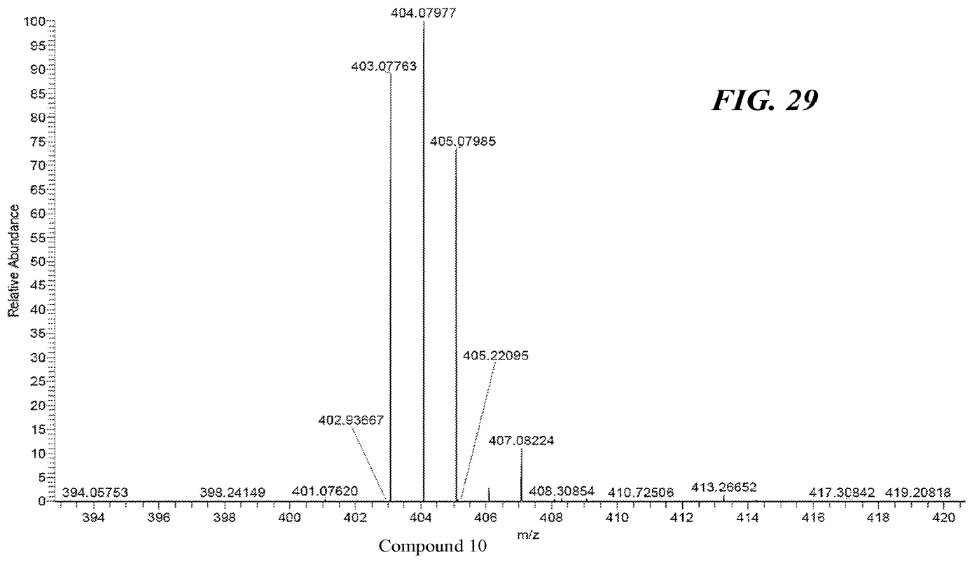

TLC: Rf 0.1 tailing on silica-gel, 1% NaCl in water, UV254 and ninhydrin. ESI-MS (positive ion, mobile phase: acetonitrile and water), calculated for [C₈H₂₂N₂PtS₂]²⁺: 404.079402; found: 404.07977. Mass spectrum data of compound 10 is shown in FIG. 29 (using the LTQ Orbitrap in electrospray ionization positive ion mode; solvent used was a 1:1 mixture of acetonitrile and water; m/z of 404.07977 was obtained with an error of 0.91 ppm).

Compound 11: To generate compound 11, Na₂[PtCl₄]·nH₂O (n~3) (134 mg, 0.307 mmol) was dissolved in deionized water (1.5 mL) before addition of 2-(methylthio)ethylamine (57 μL, 0.61 mmol). The resulted heterogenous mixture was stirred at ambient temperature overnight before an equal volume of 1,4-dioxane was added as a co-solvent and stirred at 35-40° C. A small number of precipitates formed including platinum black particles and were removed by centrifuge. The supernatant was evaporated to dry using Smart Evaporator at 40° C. The solid residue was washed with ethanol (1.5 mL, twice) followed by diethyl ether (1.5 mL, twice), and dried in vacuo to afford compound 11 (90 mg) as a light gray solid.

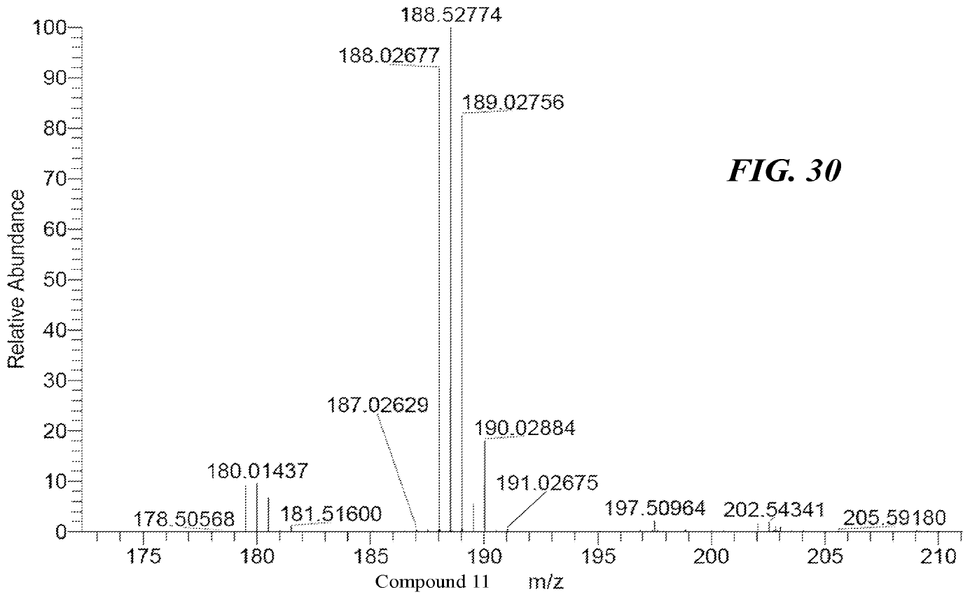

TLC: two spots, Rf ~0.1 tailing and 0.2 tailing, 1% NaCl in water, UV254 and ninhydrin. ESI-MS (positive ion, mobile phase: acetonitrile and water) calculated for [C₆H₁₈N₂PtS₂]²⁺: 188.5279635; found: 188.52774. Mass spectrum data of compound 11 is shown in FIG. 30 (using the LTQ Orbitrap in electrospray ionization positive ion mode; solvent used was a 1:1 mixture of acetonitrile and water; m/z of 188.52774 was obtained with an error of −0.08 ppm).

Table 2 provides a summary of the data related to the platinum compounds hereof with respective m/z determined by an LTQ Orbitrap in either negative or positive ion mode (solvent used was 1:1 acetonitrile and water).

TABLE 2
Summary of m/z determined by LTQ Orbitrap for Compounds 3-11

| | | Predicted | | |
|---|---|---|---|---|
| | | Monoisotopic | | |
| ID | Empirical Formula | MW (g/mol) | Predicted m/z | Measured m/z |
| 3 | [(H₃N)₂PtCl(DMSO)]⁺ | 342.0001 | 342.000673 | 342.00084 |
| 4 | Na⁺[PtCl₅(DMSO)]⁻ | 447.8235 | 447.822986 | 447.82334 |
| 5 | Na⁺[PtCl₃(DMSO)]⁻ | 377.8858 | 377.885281 | 377.88677 |
| 6 | ⁺[(H-Met-OH)₂Pt]²⁺ | 493.0669 | 246.5334435 | 246.53341 |
| 7 | [(H-(S-Me) Cys)-OH)₂Pt]⁺ 2Cl⁻ | 465.0356 | 232.5177935 | 232.51769 |
| 8 | [(Ac-Met-OH)₂PtCl₂] | 647.0257 | Not identified | Not identified |
| 9 | [(H-Met-NH₂)₂Pt]²⁺ 2Cl⁻ | 491.0988 | 490.09103 | 490.09107 |
| 10 | [(CH₃SCH₂CH₂CH₂NH₂)₂Pt]²⁺ 2Cl⁻ | 405.0872 | 404.079402 | 404.07977 |
| 11 | [(CH₃SCH₂CH₂NH₂)₂Pt]²⁺ 2Cl⁻ | 377.0559 | 188.527935 | 188.52774 |

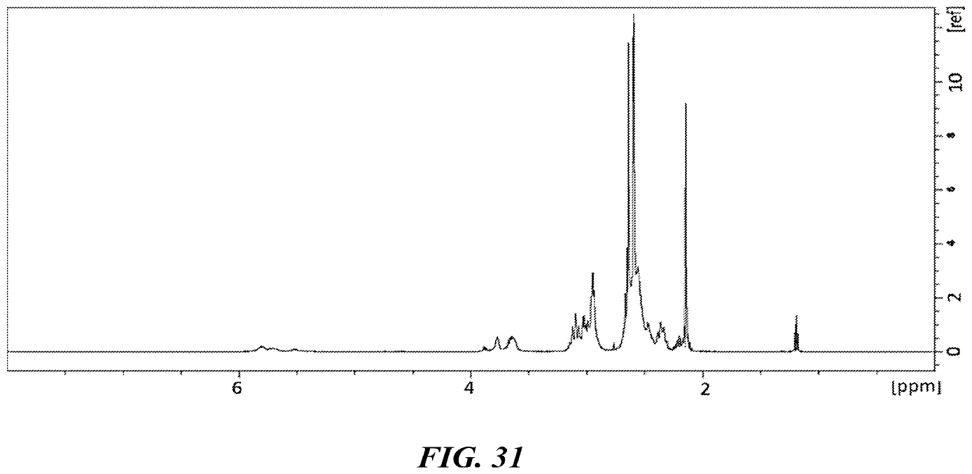

For freshly prepared samples of compound 6 with pH 5.7, ¹H NMR indicated the complex was almost exclusively cis, without detectable trans (FIG. 31; the absence of clear signals between 3.33 ppm and 3.5 ppm suggests that compound 6 is dominated by cis isomers, without any appreciable amount of trans). A 9:1 mixture of cis to trans forms was identified after 6 days of storage at room temperature by ¹H NMR. ¹⁹⁵Pt NMR resonances of the cis form are reported to be centered in the upfield region of the trans, but were difficult to clearly define at 11.7 T. The pH dependence of compound 6 ¹⁹⁵Pt NMR indicated open ring forms dominated at low pH. In contrast, the closed forms were dominant at pH>5.7. The ¹⁹⁵Pt NMR spectra for compound 8 in aqueous solutions indicated a significant degree of heterogeneity with at least 4 distinct species. Compound 8 had sulfide ligation but was anticipated to be an open form due to the acetylation of the amine site. Furthermore, the mass spectral analysis of the material was not indicative of a single component. This distinction served as basis for comparing the role of bidentate ligands.

### Example 4

### Compound Water Solubility Study

The water solubilities of the compounds were assessed as a second criterion for selecting candidates using methods commonly known in the art.

Neutral complexes resulted in substantially reduced solubilities in aqueous conditions and thus, limited utility. For instance, a 1:1 mol ratio mixture of L-methionine and Na₂[PtCl₄] in water generated an insoluble precipitate, which was identified as a mono-adduct (H-Met-OH)PtCl₂, a known neutral Pt(II) complex. Compounds 3-5 had solubilities of >50 mg/mL in water, making them all suitable for pharmacological evaluations. Compound 6 had solubilities of about 700 mg/mL (0.8M) in water at 25° C.

In contrast, the mixture with 2 mole equivalents of L-methionine in water was homogenous, indicating that the mono-adduct rapidly reacts with methionine to generate a bis-adduct compound 6 as per Scheme 1 shown in FIG. 2.

The solubility of compound 4 was also assessed in phosphate buffer. Briefly, 100 mM of compound 4 in 20 mM pH 6.0 phosphate buffer was monitored for up to 18 days. The sample was shieled from light during storage to prevent accelerated decomposition due to light exposure. An equilibrium was established after day 4 with approximately 70% of bound DMSO on platinum released.

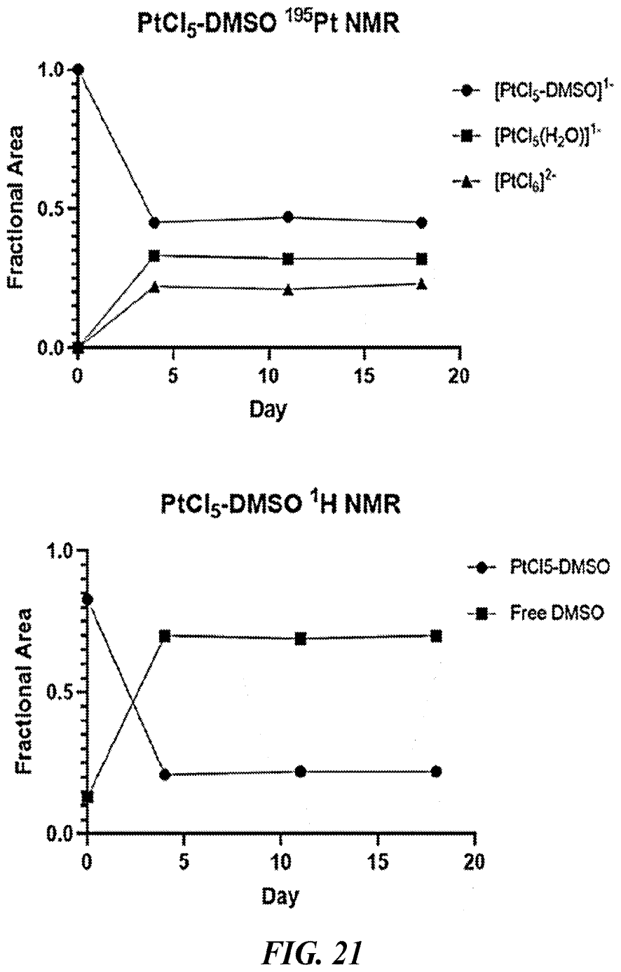

The DMSO-Pt interaction in compound 4 exhibited poor stability in aqueous solutions as monitored by NMR (FIG. 21). In contrast, the Pt compounds hereof comprising bidentate thioether ligands (e.g., compounds 6, 7, and 9-11) such as methionine were consistently stable in solutions and maintained reactivity towards cyanide. Those compounds with the capacity to form ring-closed species (e.g., compounds 6, 7, and 9-11) were also the most active cyanide scavenging agents. At higher pH, these compounds lacked a positive charge on the primary amine allowing electron donation to the platinum d-orbitals, which can contribute to resistance to hydrolysis. At lower pH values, the positively charged amine likely induces electronic repulsion to the platinum core, favoring ligation of hydroxyl or chloride.

### Example 5

### Platinum Content and Reactions with Cyanide

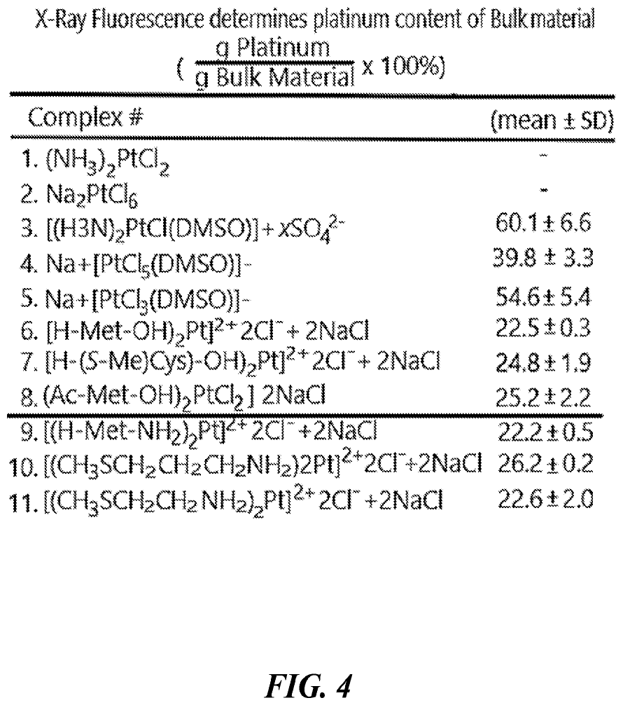

Quantification of platinum content (% w/w) in each of the sulfur-containing compounds were obtained by x-ray fluorescence spectrometry (XRF). Example XRF spectra are shown in FIG. 3, where the Pt(IV) and Pt(II) core excitations are distinct from the internal manganese standard. The XRF results revealed that the bulk materials for compounds 6-11 were 20-30% w/w Pt as summarized in FIG. 4. These values were used as the basis for available platinum mass in each animal dose, regardless of any anticipated speciation.

Each of the sulfur-containing Pt complexes' (compounds 1-11) capacities to act as cyanide scavengers were evaluated using in vitro reactions with potassium cyanide (KCN). Direct consumption of cyanide in solutions was detected using three different methods.

An ion-selective (cyanide) electrode (ISE) assay was used to evaluate the consumption of free cyanide in the presence of Pt(II) complexes. After the addition of the platinum-containing materials at 50 μM in 0.5 mM cyanide pH>10 with 0.1 M ionic strength, the time before reading was limited to 10 minutes to better mimic conditions for rapid scavenging. A summary of the amount of cyanide reduction per mole of platinum present is shown in FIG. 5. Time-dependent ultraviolet-visible spectroscopy (UV-Vis) observations of the same Pt samples in a Pt:cyanide ratio of 1:10 provided estimates of the relative rates for starting material disappearance. The product of these Pt(II) compounds reactions with excess cyanide was expected to be Pt(CN)₄²⁻. Formation of a highly stable Pt(CN)₄²⁻ was based on strong field ligand properties from cyanide, exhibiting strong d-orbital splitting of the platinum core. For each of compounds 1-11, the sulfur, amino, and chloride ligands were anticipated to be displaced by free cyanide.

As shown in FIG. 5, compounds 6, 7, 10, and 11 all appear to approach the expected consumption of 4 mole equivalents of cyanide. Product formation was also observed at 255 nm in the UV-Vis assay for compounds 3-11, which is consistent with formation of Pt(CN)₄²⁻.

ISE assay conditions were maintained at highly alkaline conditions at high ionic strength to maintain cyanide in solutions. At longer incubation periods (>10 minutes), compound 4 showed a larger fraction of cyanide consumption (Table 3), indicating incomplete scavenging in the ISE assay after 10 minutes.

TABLE 3
ISE data collected of compound 4.

| | | Free cyanide |
|---|---|---|
| | Assay | removed by platinum |
| | time | Total CN⁻-free CN⁻ |
| Complex | (min) | Moles Pt |
| #4. 2 [(H₃N)₂PtCl(DMSO)]⁺ SO₄²⁻ | 10 | 1.7 ± 0.2 |
| #4. 2 [(H₃N)₂PtCl(DMSO)]⁺ SO₄²⁻ | 90 | 2.9 ± 0.2 |

Compounds 8 and 9 were less reactive under the same alkaline conditions. The sulfur effect on the reactions with cyanide is highlighted by the reduced scavenging for compounds 1 and 2 (FIG. 5). ISE assay for 10 minutes also revealed that the sulfide complexes (compounds 6, 7, 10, and 11) were superior to the DMSO complexes (compounds 3-5) for scavenging cyanide. Similarly, UV-Vis's time-dependent changes support that the most active compounds in the ISE assays also had faster apparent rates of reaction. Compounds 6, 7, 9, 10, and 11 had 10-100 fold faster observed pseudo first order reaction rates over the DMSO complexes under like conditions some of which exceed the limit of detection.

A HPLC method was developed to monitor the direct production of Pt(CN)₄²⁻ from reactions of compound 3 and 5-11, and cyanide (Table 4) using the protocol described in Behymer et al. (2022), supra. Four biological replicates were performed. PtCl₆⁻²: DMSO exhibited an EC₁₀₀ of 62 μM.

TABLE 4

| Molar Ratio | X = Methionine | X = 3-(methylthio)propylamine |
|---|---|---|
| PtCl₆⁻²:X | EC₁₀₀ (μM) | EC₁₀₀ (μM) |
| 1:1 | 62 | 62 |
| 1:2 | 31 | 31 |
| 1:5 | 31 | 16 |
| 1:10 | 31 | 16 |
| 1:25 | 16 | 16 |
| 1:50 | 16 | 16 |

The decarboxylated form of methionine (3-(methylthio)propylamine) was effective at enhancing efficacy of hexachloroplatinate. Additionally, less appeared to be required to achieve the same antidote efficacy as compared to the carboxylated form L-Methionine.

The example data for the reaction of compound 6 with cyanide are shown in FIG. 6A with the blue trace starting material and red trace after addition of 4 equivalents of KCN. Compound 6 and Pt(CN)₄²⁻ have discrete retention times and the spectrum for compound 6 (FIG. 6B) had no appreciable absorbance at 260 nm where the product of a cyanide reaction showed a charge transfer band (FIG. 6C). The HPLC data demonstrate depletion of compound 6 as a direct correlation to Pt(CN)₄²⁻ production (FIG. 6D). These titrations were repeated for reach of the compounds and only product (Pt(CN)₄²⁻) was quantified. Results were normalized to maximum Pt(CN)₄²⁻ observed over the titration range and are summarized in FIG. 5.

Pt(IV) compounds were problematic due to poor compatibility with the column chemistry. Using the HPLC conditions, compounds 6-9 exhibited the most significant capacity for cyanide scavenging. Close inspection of the HPLC chromatograms for compounds 10 and 11 indicated evidence of low levels of intermediates, a possible explanation for reduced conversion to Pt(CN)₄⁻².

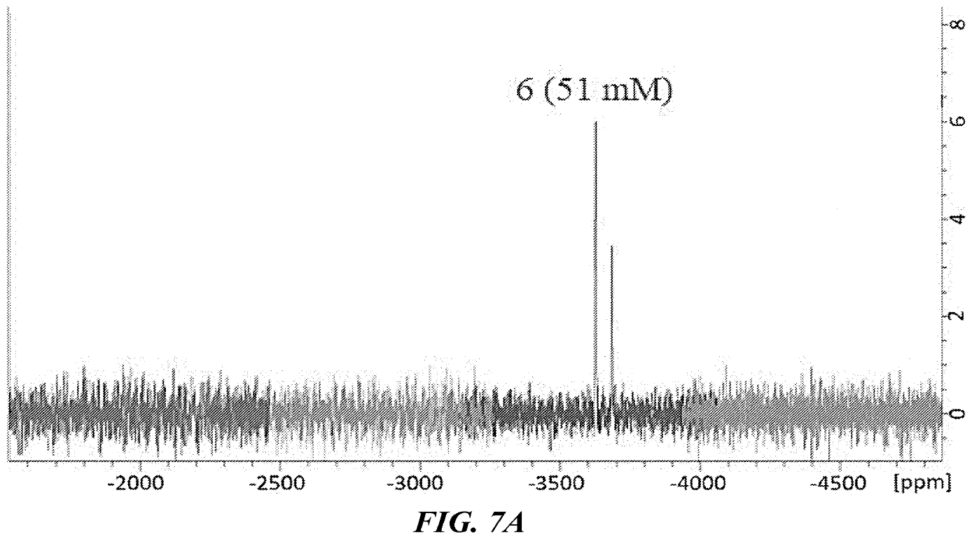

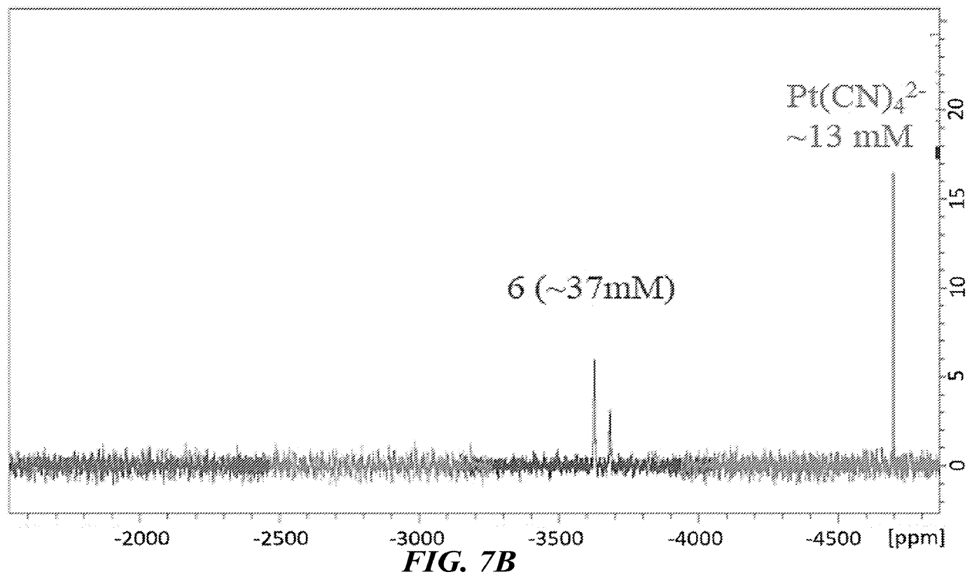

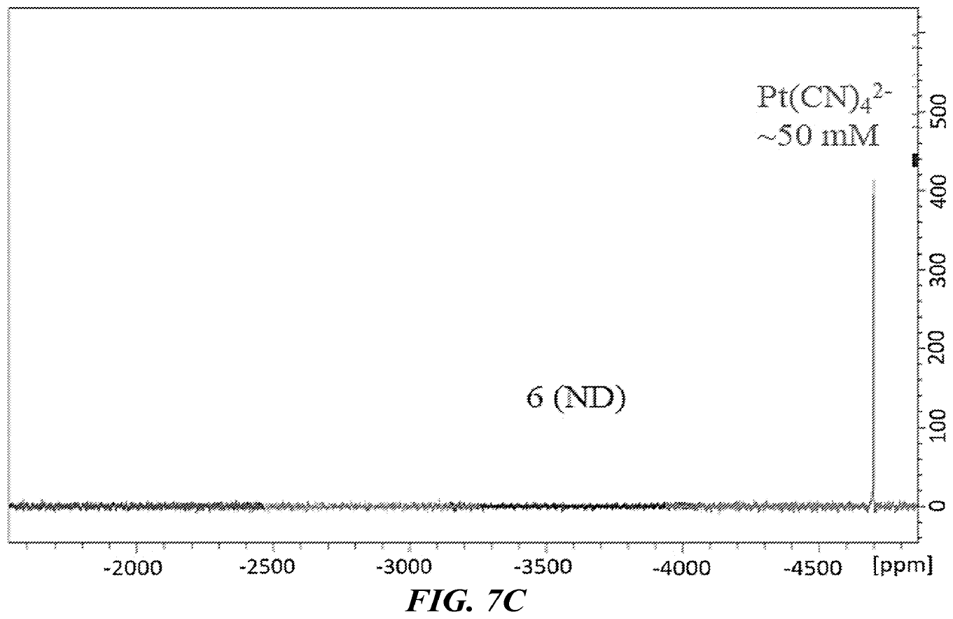

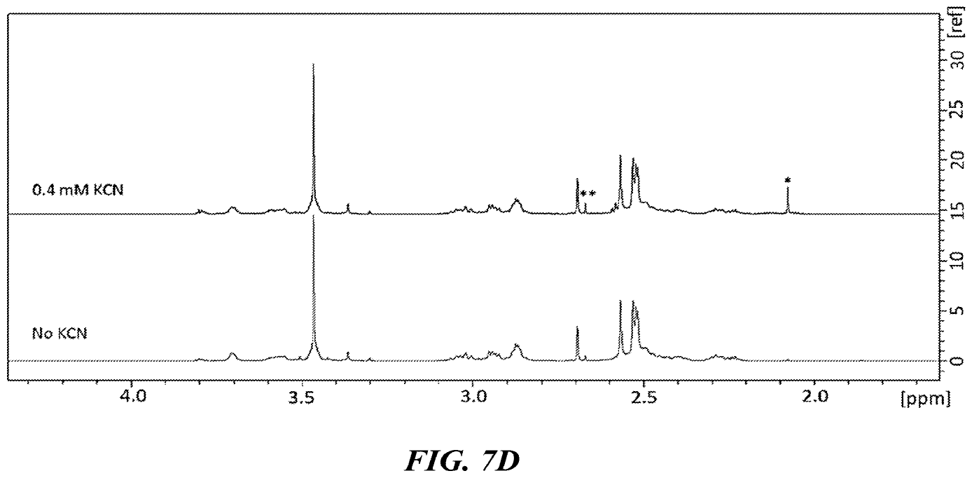

A third approach to analyzing the cyanide reactions with Pt compounds 3-11 incorporated proton nuclear magnetic resonance (¹H NMR). The results were consistent with published observations; sulfide compounds showed pH-dependent spectra resulting in ring-opening and closing in the bidentate forms. The ¹⁹⁵Pt NMR spectrum for compound 6 at pH 7.2 showed two broad peaks (FIG. 7A). The addition of 1 molar equivalent of KCN resulted in conversion to Pt(CN)₄²⁻ (FIG. 7B) in a ratio consistent with no mixed ligand intermediates. Finally, the addition of 4 equivalents of KCN resulted in quantitative conversion of compound 6 to Pt(CN)₄²⁻ (FIG. 7C).

Further, a competition reaction of 1 mM compound 3 and 1 mM compound 6 with 0.4 KCN in 180 mM NaPi, pH 7.6, 10% D₂0 buffer at 298° K, was monitored by ¹H NMR (FIG. 7D). The apparent rate of methionine release was estimated to be about ten times that of DMSO.

Interestingly, these data are consistent with the direct conversion of the amino-sulfide Pt(II) complexes to Pt(CN)₄²⁻ without the measurable existence of any mixed ligand intermediates. Instead Pt(CN)₄²⁻ was the only Pt(II) product under equal molar amounts of cyanide and Pt(II) compound. The implication is that the addition of the first cyanide ligand could be rate-limiting. This feature could be an essential attribute for enhancing the efficacy of Pt(II) cyanide scavenging agents.

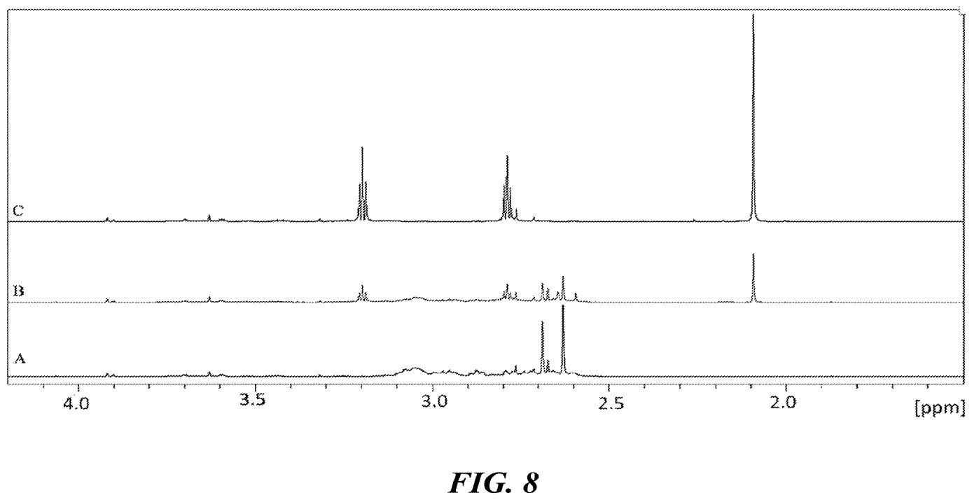

Each complex was also titrated with cyanide and monitored by ¹H NMR (with a representative example for compound 11 in FIG. 8), with A showing 1 mM compound 11 alone in 50 mM NaPi, pH 7.5, 10% D₂O; B showing 1 mM KCN added; and C showing with 5 mM (final concentration) KCN added. Ligand (2-methylthioethylamine (MTEA)) was easily identified by the characteristic sharp peaks at 2.69 ppm and 2.62 ppm for the bound form, and 2.1 ppm for the free form. In the absence of KCN, MTEA was found to be fully bound to Pt. The addition of KCN lead to quantitative release of MTEA, as the signal at 2.1 ppm became incrementally higher in B and C of FIG. 8. Complete disappearance of bound ligand is observed in C of FIG. 8.

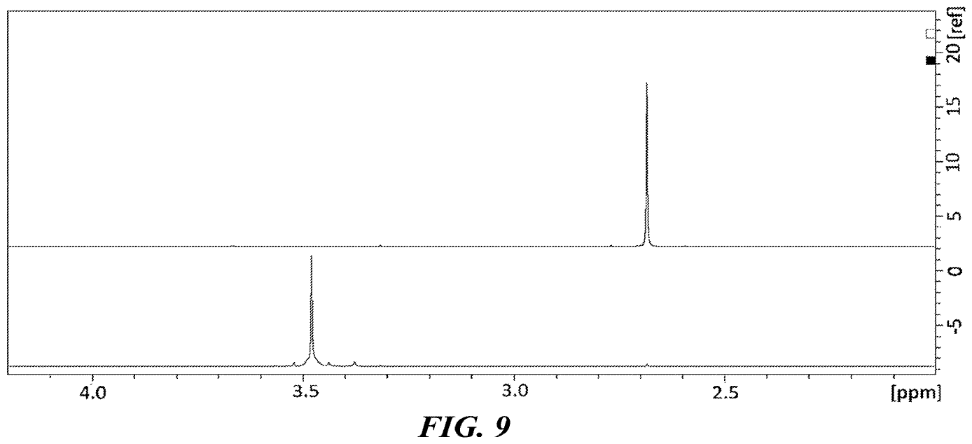

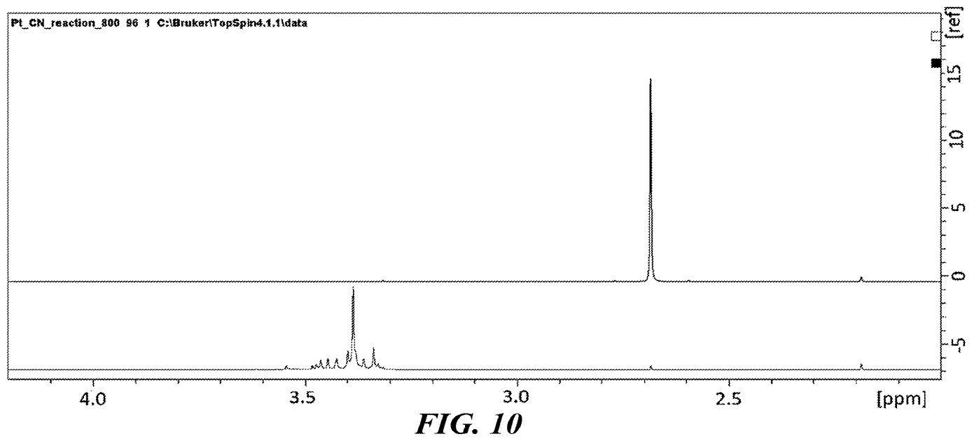

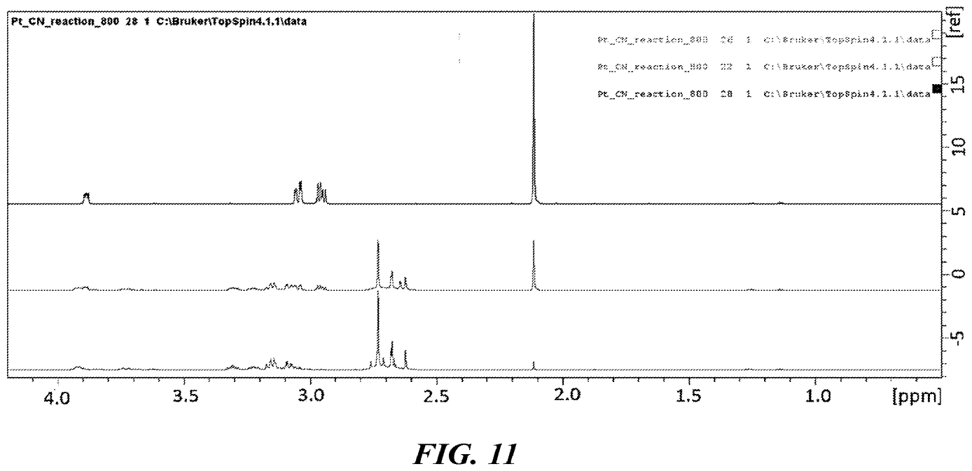

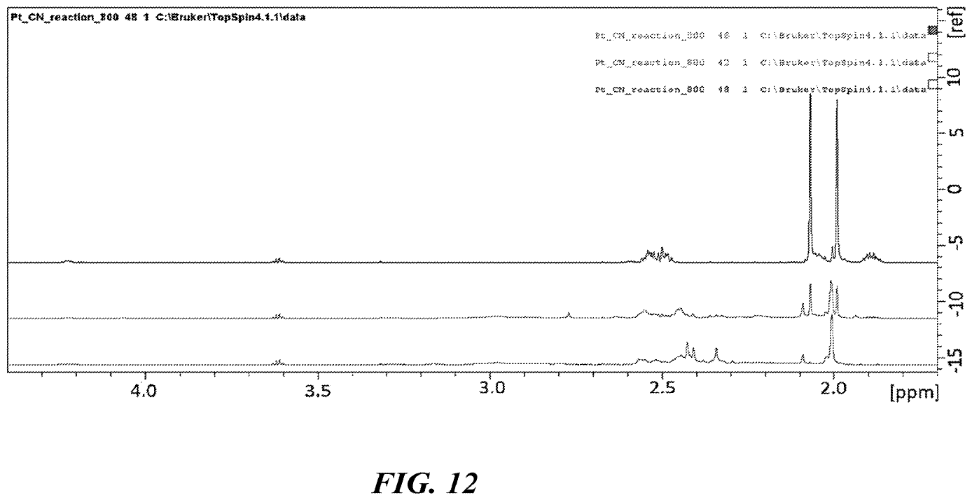

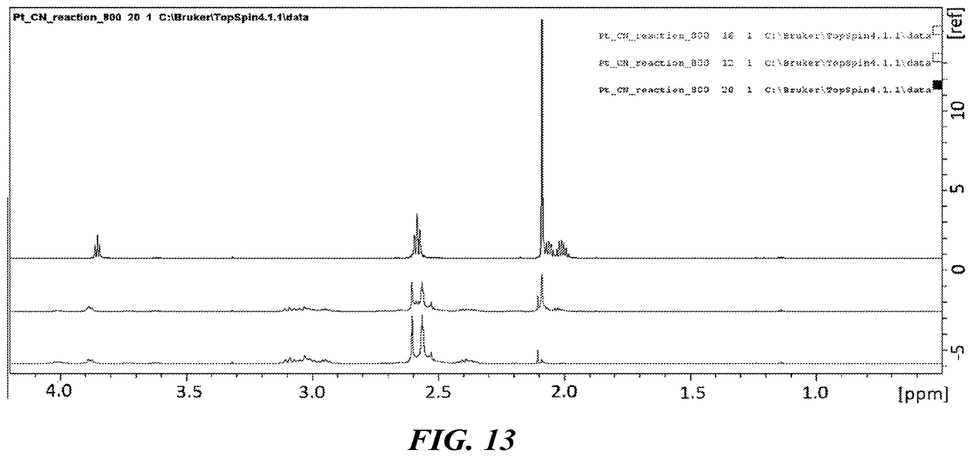

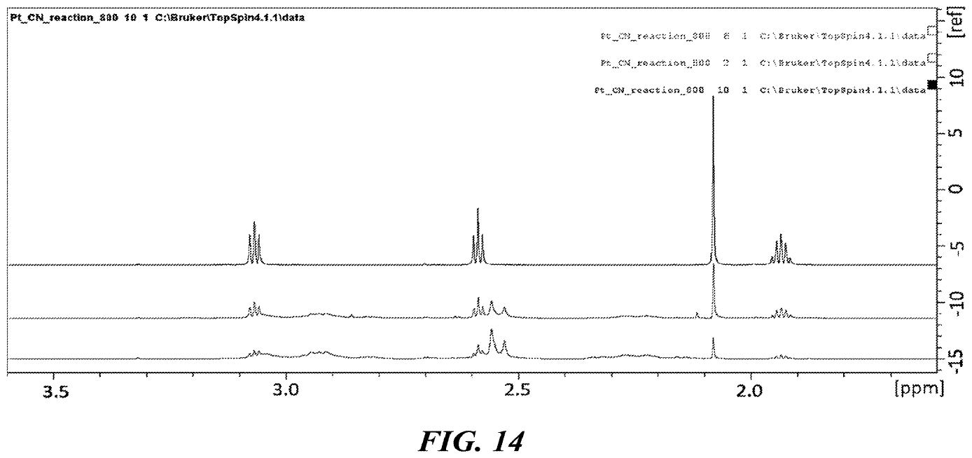

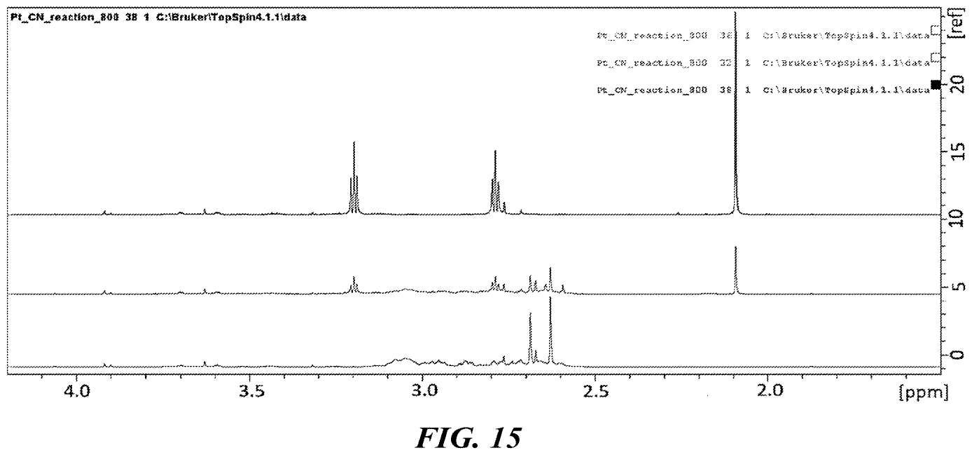

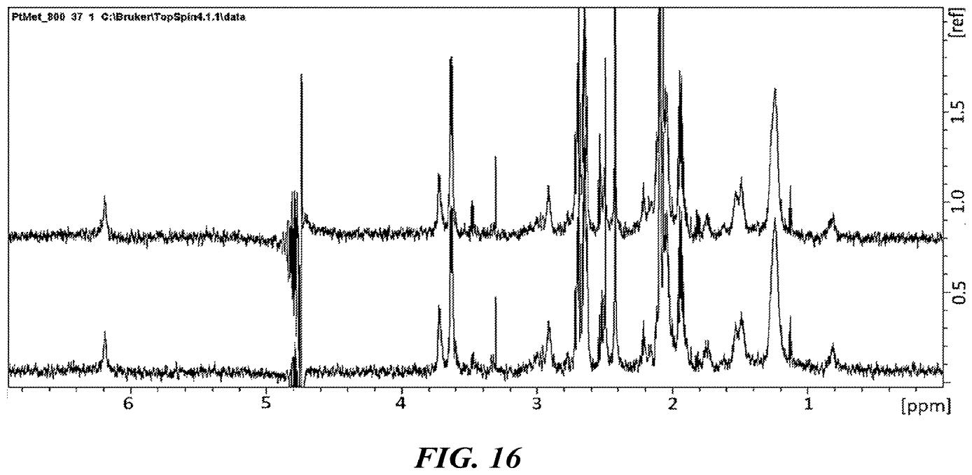

The release of amino sulfide or DMSO ligands can be readily observed as they have distinctive sets of chemical shifts and line shapes. All the reaction materials in compounds 6-10 showed results consistent with the direct displacement of ligands by cyanide (FIGS. 9-15). Similarly, further investigation of the reduced cyanide scavenging of compound 9 under alkaline ISE conditions showed low conversion to product by proton NMR (FIG. 16).

The exchange of proteins with Pt(II) metallodrugs remains an area of active investigation. Several studies have examined the interactions of amino acid side chains with platinum drugs. Components of biological matrices that ligate platinum, such as thiol-containing metabolites such as glutathione, may reduce platinum's capacity to scavenge cyanide.

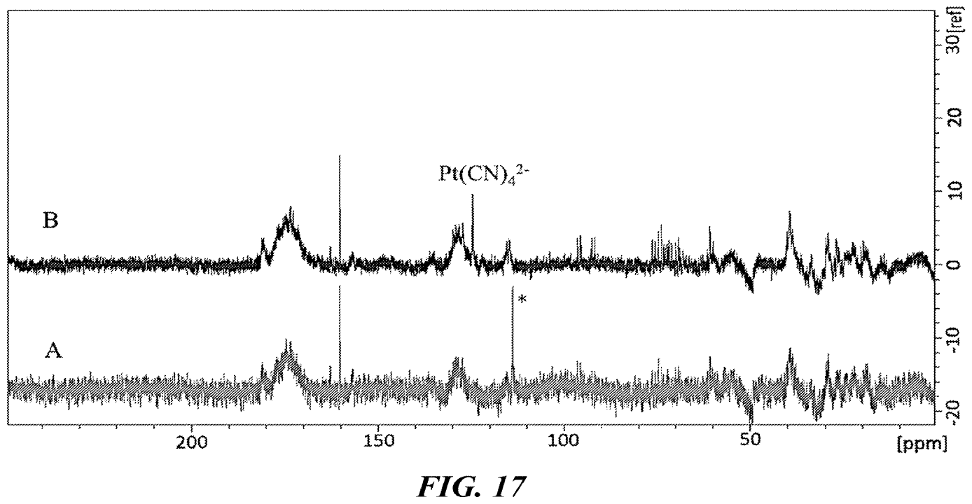

Reactions were executed in the presence of rabbit serum to assess the impact of biological matrices on the cyanide reactivity of compound 6 (FIG. 17). As a feasibility study, 100 μM of compound 6 was exposed to 400 μM [¹³C]KCN in 100% rabbit serum, and ¹²C NMR spectra were acquired (FIG. 17). Semi-quantification of ¹³C in serum revealed that Pt(CN)₄²⁻, with a signature chemical shift at 125 ppm, was the major reaction product within <1 hour of mixing (if not shorter, due to the required NMR data collection time). The implication is that the serum matrix does not significantly reduce the ability of compound 6 to scavenge cyanide as compared to serum-free conditions.

### Example 6

### Zebrafish Cyanide Rescue Studies

Zebrafish were used to assess the sulfur-containing Pt compounds 3-11 and prioritize candidate agents for further in vivo testing (e.g., in rodent models of cyanide toxicity). Briefly, zebrafish larvae were plated into 96-well plates at 6 days post fertilization in HEPEs buffered Tubingen E3 medium (n=5 per well). Compounds 3-11 were reconstituted in purified water and larva was dosed with potassium cyanide (50 μM) and each platinum complex (1-250 μM) in each well (until 100% rescue was obtained; see Table 5). This cyanide concentration represented the lethal dose (LD100) for all embryos in each well and survival was documented after 4 hours of exposure post-treatment.

TABLE 5
Efficacy of countermeasures in cyanide challenged Zebrafish

| Complex | ${EC}_{100} \frac{μg Pt}{mL}$ |
|---|---|
| 1 | 2.9^{b} |
| 2 | 12.1^{b} |
| 3 | 3.3 |
| 4 | 11.7 |
| 5 | 3.3 |
| 6 | 1.2 |
| 7 | 1.3 |
| 8 | 4.8 |
| 9 | 4.6 |
| 10 | 2.4 |
| 11 | 1.9 |

Zebrafish data represents EC₁₀₀ of each compound formulation with aqueous conditions.
^{b} = platinum compound was pre-dissolved in DMSO.

Each result is the concentration of platinum necessary for n=5 (100%) survival in the presence of 100 μM KCN which resulted in death after 1 hour in the control groups. Reported survival rate was 4 hours after cyanide exposure.

Toxicity in zebrafish is represented as the lethal dose in half the population (LD₅₀) which is due to the presence of the platinum compounds hereof Toxicity is represented as a percentage heart rate of the control group; lower heart rates indicate increased cardiotoxicity (n=12 per group).

When compiled on a per mole Pt dose formulated in DMSO, cisplatin (compound 1) was more potent than hexachloroplatinate (compound 2). The same trend was observed for the isolated DMSO compounds 3 and 4, which were equipotent with the formulations made in DMSO. These results are consistent with the active Pt agent formulations comprising one DMSO ligand. This analysis further demonstrates that the Pt(II) compounds hereof exhibit greater efficacy than Pt(IV) complexes. Also included for comparison is the isolated Pt(II)-DMSO compound 5, which showed efficacy similar to compound 3. In addition to the sulfur-directing effect for cyanide substation to Pt, the compounds 3-5 all exhibited improved aqueous solubilities consistent with contributing to the overall cyanide rescue properties. When tested using the same zebrafish model, the amino-sulfide Pt(II) compounds 6-11 revealed similar trends, with compounds 6, 7, 10, and 11 exhibiting potencies comparable to cisplatin-DMSO compound 3.

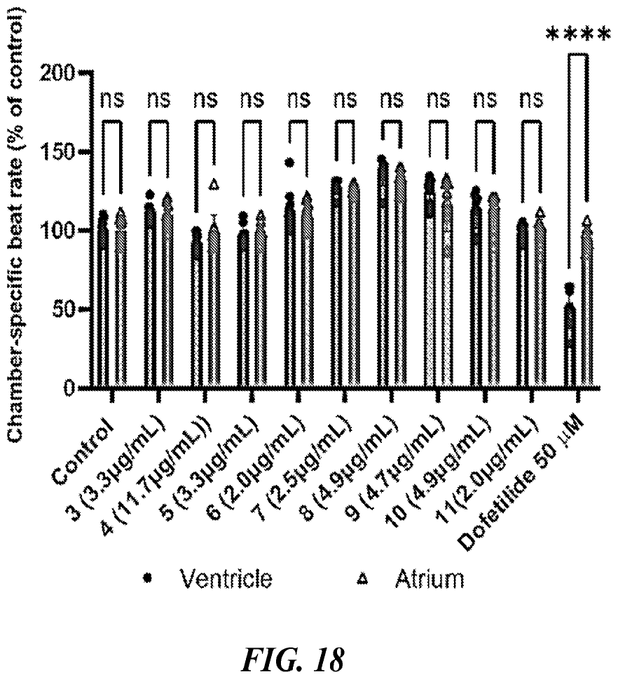

To assess cardiotoxicity, heart rate and atrioventricular (AV) conduction were used to screen each compound without cyanide in a validated physiological analysis (FIG. 18). Burns et al., High-throughput assay for small molecules that modulate zebrafish embryonic heart rate, Nature Chemical Biology 1: 263-264 (2005); Milan et al., Drugs that induce repolarization abnormalities cause bradycardia in zebrafish, Circulation 107: 1355-1358 (2003). Analysis was performed using a multiple comparisons test between means of ventricle and atrium.

Briefly, TubigenAB zebrafish embryos (bred in house) were incubated for 2 hours with each compound at indicated dose (target concentrations/doses were chosen from the zebrafish cyanide rescue efficacy study described above) or a vehicle alone (used as a control). The heart rate was normalized to the control group. Additionally, dofetilide was used as a positive control to demonstrate AV 2:1 block. Heart rate was measured in 15 second intervals by video. Atrial and ventricular heart rates were calculated from the average pixel density over time in the region of interest. Fast-Fourier Transform was performed to determine heart rate and AV concordance was estimated.

The differences between the two means were not significant for all doses administered; there was no evidence of bradycardia or AV block observed using the EC₁₀₀ doses determined for compounds 1-11.

### Example 7

### Mouse Inhalation Studies

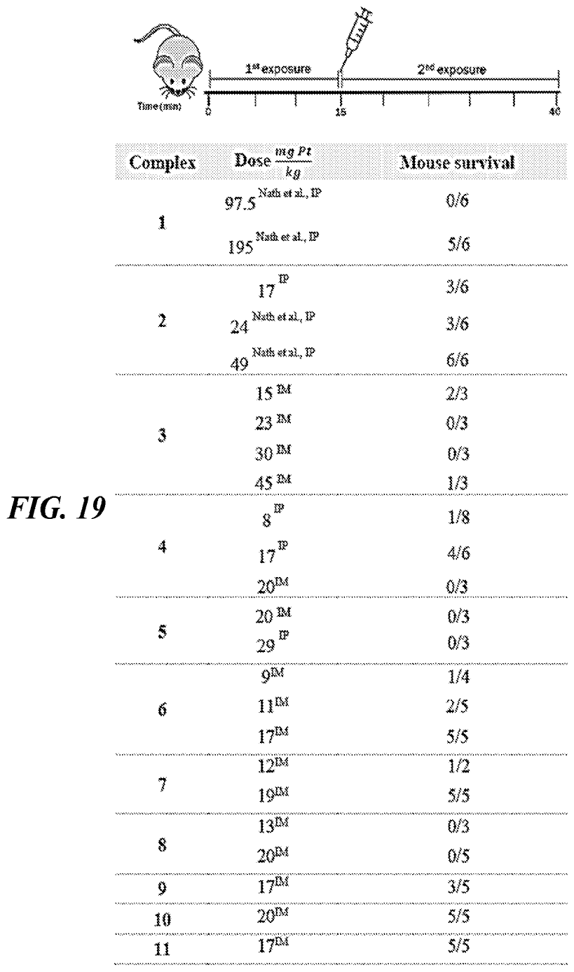

While the in vivo zebrafish testing indicated the potential for these Pt(II) compounds to serve as cyanide scavengers, in certain embodiments, it would be beneficial for availability through intramuscular injection (IM). The lethal inhalation model in mice offers a stringent test for efficacy using either intraperitoneal (IP) or IM administration. The basic scheme for this established model is presented in FIG. 19. This model has also been used to test the efficacy of conventional platinum-based cyanide countermeasures. Thompson & Marrs, Hydroxocobalamin in cyanide poisoning, Clinical Toxicology 50: 875-885 (2012); Summa et al. (2006), supra.

Briefly, mice were placed in a gastight chamber and exposed to a lethal concentration of cyanide gas (LD₁₀₀ after 40 minutes). After 15 minutes the mouse was removed, injected with antidote IM, and placed back into the cyanide chamber for an additional 25 minutes. A phosphate buffer was used to control the pH in each formulation of the different sulfur-containing Pt compounds. This model represents a real-life scenario of people being exposed to cyanide gas in a difficult-to-access enclosed space such as a factory or subway station, with 15 minutes required for emergency medical personnel to arrive at a disaster scene and 25 minutes required to simultaneously treat and evacuate the victims.

Despite the potency of compound 3 in the zebrafish model of Example 6, the mouse model revealed that the material was not reproducibly effective when administered by IM injections (FIG. 19). This result is consistent with the previous studies with the cisplatin formulated in DMSO.

When compound 4 was delivered IP, the efficacy was consistent with that observed for compound 2. Nath et al. (2017), supra; Morningstar et al., Intramuscular administration of hexachloroplatinate reverses cyanide-induced metabolic derangements and counteracts severe cyanide poisoning, FASEB Bioadvances 1: 81-92 (2019). However, similar to compound 3, no efficacy was observed with the DMSO compounds 4 and 5 when dosed in the cyanide treated mice up to 20 mg/kg by IM administration. Previously, compound 2 was found to be active by IM injection in a lethal cyanide rabbit model at 10.4 mg/kg Pt (equivalent to 41.6 mg/kg in mice). However, the potential for dose limiting muscle toxicity was also observed at 69 mg/kg Pt dose in mice, indicating a potentially narrow therapeutic window.

Platinum amine-sulfide containing compounds (compounds 6-11) showed a contrasting profile to the DMOS compounds when tested by IM administration. For compounds 6 and 7, the efficacy in the mouse model showed robust rescue at 17 mg/kg and 19 mg/kg, respectively (FIG. 19). Similarly, compounds 9-11 (analogs of compounds 6 and 7) showed similar potencies using IM administration. These results indicate a significant enhancement in the antidote activities for the five amine-sulfide Pt compounds, relative to the DMSO Pt compounds 2, 3, and 4.

In contrast, compound 8 appeared to have no cyanide counteractivity in the mouse model using the same IM dose range. One explanation for this loss in activity is the observed heterogeneity of the Pt species in solution, which can decrease bioavailability.

Compound 9 also appears to be a less efficient agent than compounds 9-11. The results are consistent with the hypothesis that sulfur ligands on platinum can enhance the efficacy for platinum as a cyanide countermeasure. However, it is likely the physiochemical properties of the Pt ligands impart critical attributes to enable bioavailability to the IM dose form.

Further, stability of an efficacious dose of compound 6 was verified by aging the formulation of compound 6 for 7 days with subsequent use in the above-described lethal inhalation mouse model. Briefly, solutions of compound 6 were tested in the mouse inhalation model after standing at ambient temperature for up to 7 days. The same dose was used in each of 4 studies, each with 3 mice, where the dose was administered at days 0, 1, 3, and 7. In all cases, survival was observed, supporting compound 6 could be an effective cyanide countermeasure candidate with a useful shelf-life (Table 6).

TABLE 6
Efficacy of compound 6 in aged samples.

| Sample age (days) | Survival |
|---|---|
| 0 | 3/3 |
| 1 | 3/3 |
| 3 | 3/3 |
| 7 | 3/3 |

In sum, efficacy improvements in Pt(II) doses relative to cisplatin-DMSO compounds were observed. The efficacious dose administered for, for example compound 6, in the mouse model scales to a dose of 51 mg/m², which is within the range of a single human dose of cisplatin used for oncology.

### Example 8

### Pharmacokinetics & Pharmacodynamics in Non-Lethal Rabbit Cyanide Exposure

The real time monitoring of the oxygenated/deoxygenated hemoglobin by diffuse optical spectroscopy allowed for a direct readout of cyanide effects in vivo. Brenner et al., Cyanide antidotes in development and new methods to monitor cyanide toxicity, In Toxicology of Cyanides & Cyanogens 309-316, John Wiley & Sons, Ltd. (2015). A platform for conducting these studies in a rabbit model for cyanide toxicity enabled a direct comparison of lead countermeasure candidates.

The rabbits were ventilated with 100% O₂ supply throughout the experiment. The sublethal amount of cyanide was infused for 55 minutes (0.167 mg/min) and the antidotes were injected intramuscularly at the completion of cyanide infusion. The change in in vivo tissue oxygenation status was monitored non-invasively with continuous wave near infrared spectroscopy (CWNIRS) for 90 minutes post antidote injection.

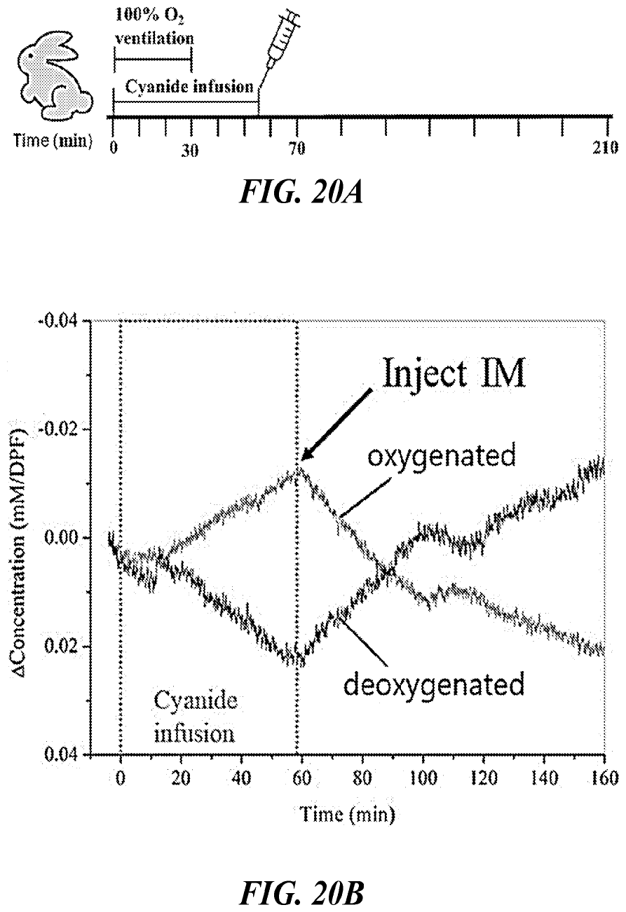

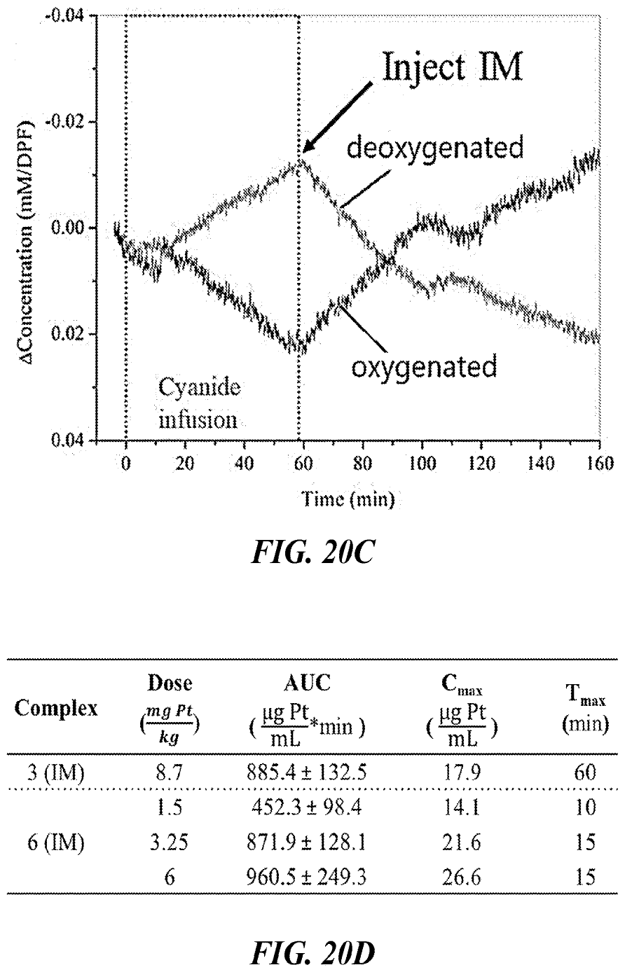

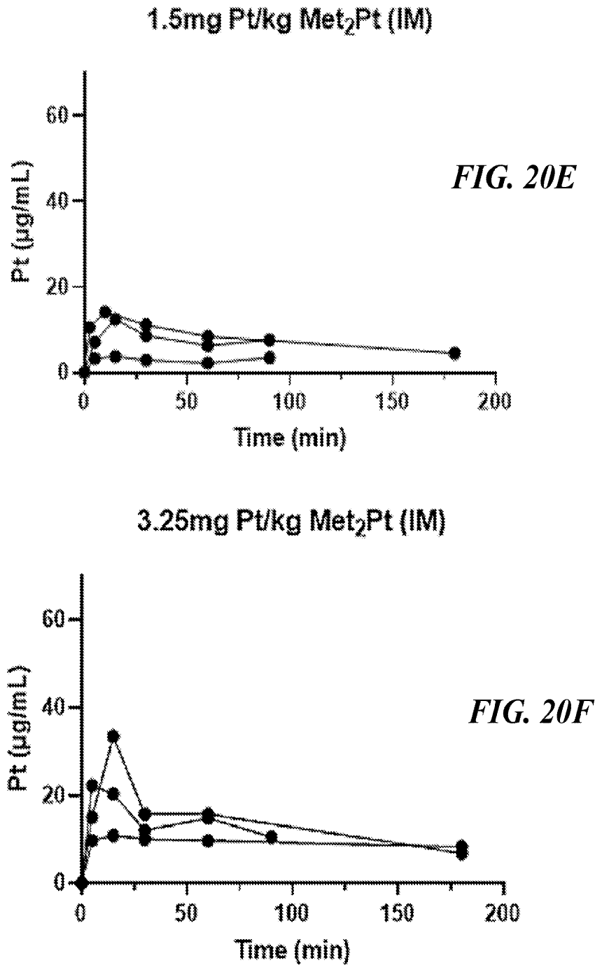

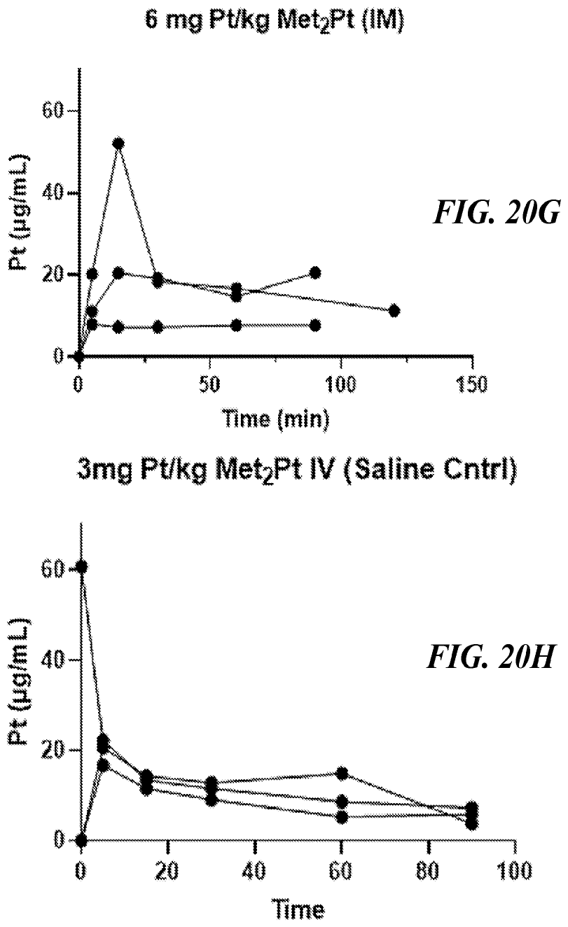

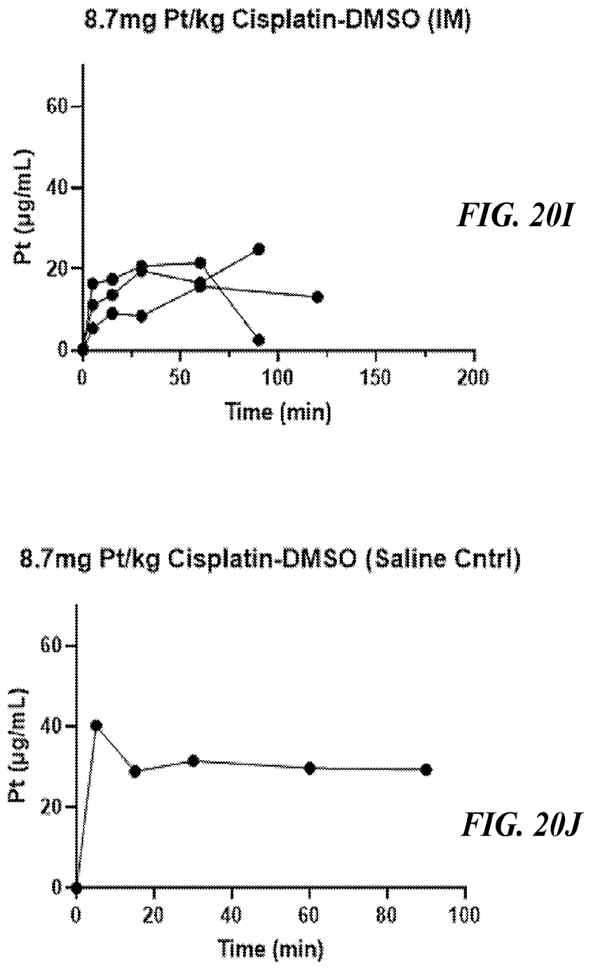

A comparison of the time-dependent changes of hemoglobin states was performed for compounds 3 and 6 (FIGS. 20A-20D). Upon IM delivery of compound 3, the rate of change of the oxygenated/deoxygenated hemoglobin ratio in blood was reestablished over the next 2 hours, representing a significant response in the non-lethal rabbit model (FIG. 20B). For compound 6, IM delivery resulted in a rapid change of the oxygenated/deoxygenated hemoglobin ratio over the next 15-20 minutes (FIG. 20C). These data are consistent with the apparent reactivity of compound 6 with cyanide using the in vitro measurements.

The significant response to compound 3 in this non-lethal cyanide toxicity rabbit model was not anticipated given the failure of IM administration to show meaningful efficacy in the mouse inhalation model. Analysis of the total platinum content in blood over the time course of the experiment was used to compare IM doses of compounds 3 and 6 as indicated by the area under the curve (AUC) (FIG. 20D). Dose normalization to total platinum injected shows that Cₘₐₓ levels were comparable at the higher doses.

Blood plasma concentrations of total platinum content were also collected by ICP-MS of rabbits dosed with compounds 3 or 6 in the non-lethal cyanide model (FIG. 20D). Blood samples were collected for up to 180 minutes and data was presented as μg/mL versus time as total platinum concentration. ICP-MS sample infusion was performed using an Arridus peristaltic pump as described herein and analysis of each sample was performed using a collection time of 90 seconds each. Platinum 195 peak intensities were converted to micrograms per milliliter as described above.

What was most distinct was the Tₘₐₓ for 8.7 mg of compound 3 versus 6 mg of compound 6 in total Pt dose. Direct comparison between Tₘₐₓ suggests that the rate of absorption for compound 6 contributed to achieving efficacy in both the mouse and rabbit models of cyanide toxicity.

### Example 9

### Site of Injection Toxicity in Mouse Model

A prior study with PtCl₆ formulated in DMSO and PBS using a 200 mg/kg IM dose in mice revealed significant levels of chemically-induced tissue damage. Morningstar et al. (2019), supra. To contrast with these previous results observed with hexachloroplatinate in DMSO, a comparative muscle toxicity study was performed to compare both platinum compounds 4 and 6, and assess if toxicity may be a general limitation of the novel Pt(II) compounds hereof Briefly, CD-1 mice were acquired from Envigo (Indianapolis, IN) at age 3-4 weeks with weight ranges of 18-20 grams. In two separate studies, each complex was administered at 42 mg/kg and 50 mg/kg to male (n=2 for each dose) and female (n=2 for each dose) via IM injection into the gastrocnemius muscle. The injection samples were 50 μL solution of compound 4 or compound 6 containing Ca²⁺ and Mg²⁺-free PBS. Studies were performed at the Purdue Translational Pharmacology and Clinical Veterinary Pathology Laboratories which with full IACUC approval. The injection sites were monitored for significant inflammation or bruising. Two cohorts of animals were humanely euthanized following the PHS Policy on the Human Care and Use of Animals at one day and 5 days after dosing. After euthanasia, the gastrocnemius muscle was surgically removed and fixed for necropsy and histopathological analysis.

In contrast to prior studies with HCP/DMSO, compound 4 at the reduced levels revealed no important pathological findings beyond the site of needle injection.

For IM injections of compound 6 at 2.5-fold or 3-fold in excess of the EC₁₀₀ in the mouse model, inducement of some acute myonecrosis in about 60% of the sections analyzed was observed, which appeared as hypereosinophilic or swollen microfibers. The effects were localized and indicated a loss of cross striation and sarcoplasmic fragmentation at day 1 post-injection with improvement in these sections at day 5. There were remaining signs of localized inflammatory and edematous changes at the injection site in approximately 40% of the sections. The compilation of information did not indicate any significant tissue toxicity beyond the injection site induced by compound 6.

### Example 10

### Formulation of Compounds 6, 9, 12, and 13

Platinum forms strong bonds with four cyanide equivalents, which enhances the potential to ameliorate the toxic effects of cyanide poisoning. Early investigations demonstrated an important DMSO solvent effect that modulates the efficacy of cyanide toxicity in a zebrafish model. Nath et al. (2017), supra. Follow up efforts establish efficacy of an IM administered Pt(IV)-DMSO complex in a lethal porcine model showing 100% rescue at 35.6 μmol/kg. Morningstar et al. (2019), supra. As described in the above Examples, it has also been determined that Pt(II)-thioether compounds derived from metabolites of cisplatin can be approximately 10-25-fold more efficacious against cyanide in lethal zebrafish exposure models than cisplatin formulated in DMSO. Behymer et al. (2022), supra. In addition, these new Pt(II)-thioether compounds (e.g., compounds 6-11) were bioavailable via IM administration providing efficacy in the mouse lethal cyanide inhalation model. Accordingly, further research into the properties of Pt(II) compounds, their characteristics, and properties was warranted.

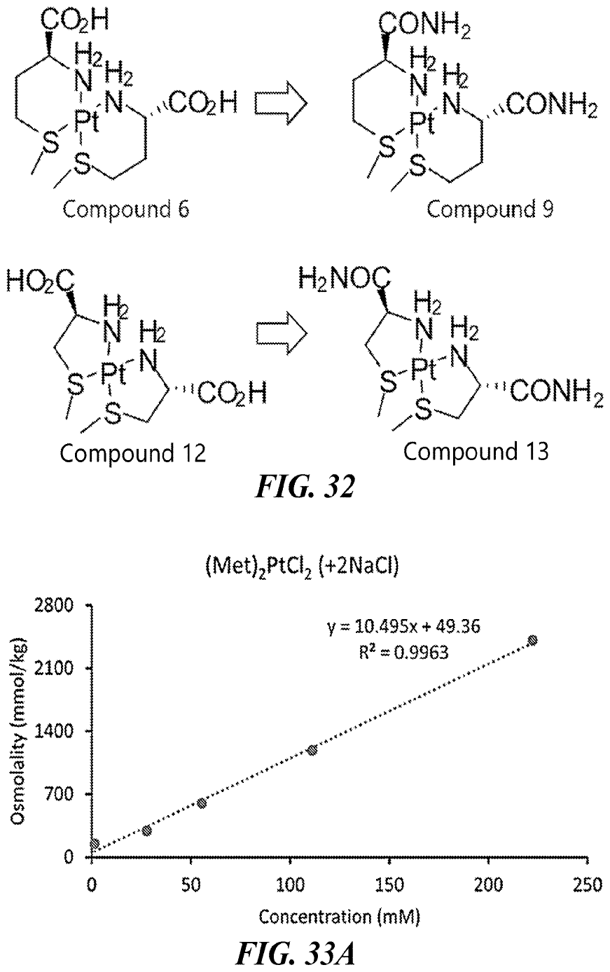

Additional compounds were prepared to serve as improved cyanide scavenging agents, including Pt(II) compounds comprising new ligands. In addition to the identification of new ligands for the Pt(II) compounds, a shelf-stable formulation would be beneficial for use in the development of a practical, safe, and effective cyanide countermeasure for field deployment. FIG. 32 shows representative structures of four bidentate Pt(II) compounds (compound 6, compound 9, compound 12, and compound 13) drawn in the cis configuration (solely for illustrative purposes). Compound 6 is a six-membered bidentate complex (or pharmaceutical salt) and, in certain embodiments, comprises bis-(L-methionine (S,N)platinum(II) dichloride. Compound 6 can be, for example, the bidentate, closed-ring configuration of compound 1 described above. Compound 9 is a six-membered bidentate complex (or pharmaceutical salt) comprising a methionine and carboxamide and, in certain embodiments, comprises bis-L-methionine amide (S,N)platinum(II)dichloride). Compound 9 can be, for example, the bidentate, closed-ring configuration of compound 2 described above. Compound 12 is a five-membered bidentate complex (or pharmaceutical salt) comprising bis-(S-methylcysteine)-(S,N)platinum(II) dichloride. Compound 13 is a five-membered bidentate complex (or pharmaceutical salt) comprising bis-(S-methylcysteine amide)-(S,N)platinum(II) dichloride. Each of compounds 6, 9, 12, and 13 can be modified with functional groups including, without limitation, one or more alkyl groups, hydroxyl groups, amino groups, carbonyl groups, carboxyl groups, and the like.

Compound 6 (+2NaCl): To generate compound 6 (+2NaCl), a mixture of Na₂[PtCl₄]·nH₂O (2.08 g, 5.20 mmol), methionine (1.59 g, 2.05 mol eq), and Milli-Q water (10.4 mL) were sonicated until all solids were dissolved, and stirred in the dark at ambient temperature overnight. Thin-layer chromatography (silica-gel 60 F254-coated) (MilliporeSigma, Burlington, MA), developed by 1% sodium chloride in water, stained by UV254, iodine and ninhydrin) was used to ensure the completion of the reaction. The mixture was centrifuged to remove platinum-black precipitates. The clear supernatant was lyophilized overnight to isolate compound 6, a pale-yellow solid (3.66 g).

Compound 6 (no NaCl): A mixture of K₂PtCl₄ (208 mg, 0.50 mmol) and methionine (75 mg, 0.50 mmol) and Milli-Q water (2.0 mL) was sonicated. The mixture quickly became a dark red-brown colored homogenous solution, and then began to generate precipitates and to change color to light brown, which was fading upon time. The mixture was sonicated to dissolve all chunks of methionine, which immediately generated precipitates, and stirring was continued at ambient temperature in the dark overnight.

The solids were isolated form the reaction mixture and washed with Milli-Q water (0.5 mL×3) to remove KCl, then with isopropyl alcohol (0.5 mL×2-times) followed by diethyl ether (0.5 mL×2) and dried in vacuo to afford mono(methionine)platinum dichloride (MetPtCl₂, 133 mg). A mixture of MetPtCl₂ (1121 mg), methionine (411 mg, 1.02 eq.), and Milli-Q water (2.7 mL) was sonicated to make it homogenous solution, kept at ambient temperature in dark overnight, and lyophilized to isolate compound 6 (1526 mg) NaCl-free batch. Platinum content for this product was approximately 38% w/w Pt (Table 7). Absorption data uses the diluent as a reference blank. Data shown is the platinum analysis acquired by UV-Vis and KCN after 24 hours. The results are averages of n=3 replicates with <0.5% RSD showing good reproducibility for the UV-Vis method.

TABLE 7
Platinum content for compound 6 (no NaCl).

| | Absorbance | Pt(CN)₄²⁻ | Pt(CN)₄²⁻ Stock | Pt % |
|---|---|---|---|---|
| Source | (255 nm) | Cuvette (mM) | Calc. (mM) | w/w |
| Na₂PtCl₄ | 0.669 | 0.0698 | 9.7374 | 37.98 |
| precipitation | | | | |
| Na₂PtCl₄ | 0.669 | 0.0698 | 9.7338 | 37.96 |
| precipitation | | | | |
| Na₂PtCl₄ | 0.674 | 0.0703 | 9.8139 | 38.27 |
| precipitation | | | | |

Analysis of percent platinum content for each replicate resulted in an average ± standard deviation of 38.1 ± 0.18

Compound 9 (no NaCl): A mixture of K₂PtCl₄ (415 mg, 1.0 mmol), methionine carboxamide hydrochloride (185 mg, 1.0 mmol), and Milli-Q water (1.8 mL) was sonicated until all solids dissolved. The mixture was kept at the ambient temperature for 5 days in the dark. White solid precipitates were generated, which was isolated by centrifuging the mixture. The solid was resuspended with ice-chilled Milli-Q water (1.0 mL), centrifuged, and supernatant was carefully removed. This water-washing procedure was repeated twice more to remove KCl. The solid was rinsed with isopropyl alcohol (1.0 mL×2) followed by diethyl ether (1.0 mL×2), dried to afford mono(methionine-carboxamide)platinum dichloride (MetNH₂)PtCl₂, 305 mg). A mixture of (MetNH₂)PtCl₂ (305 mg), methionine carboxamide hydrochloride (139 mg, 1.02 eq.), and MilliQ water (1.5 mL) was sonicated to make it homogenous solution, kept at ambient temperature in dark overnight, and lyophilized to isolate compound 2′ (452 mg) as a NaCl-free batch. Platinum content for this product was approximately 33% w/w Pt (Table 8).

The data shown in Table 8 is the platinum content analysis acquired by UV-Vis and potassium cyanide, and are the averages of 3 replicates with 32% and 33% %2/2 Pt for Batch A and batch B, respectively. These results support synthesis of compound 2′ (no NaCl) may reliably produce a solid material with consistent platinum content (absorption at 255 nm was recorded after background subtraction using the diluent as a reference).

TABLE 8
Platinum content for two batches of compound 9 (no NaCl).

| | | | Pt(CN)₄²⁻ | Pt(CN)₄²⁻ | |
|---|---|---|---|---|---|
| Preparation | | | Cuvette | Stock Calc. | Pt % |
| # | Source | Absorbance | (mM) | (mM) | w/w |
| 1^{†} | PtCl₂ | 0.799 | 0.083 | 8.31 | 32.41 |
| | PtCl₂ | 0.776 | 0.081 | 8.08 | 31.49 |
| | PtCl₂ | 0.791 | 0.082 | 8.23 | 32.10 |
| 2^{††} | PtCl₂ | 0.822 | 0.086 | 8.56 | 33.38 |
| | PtCl₂ | 0.811 | 0.084 | 8.44 | 32.92 |
| | PtCl₂ | 0.813 | 0.085 | 8.47 | 33.01 |

^{†}Analysis of percent platinum content for each replicate resulted in an average ± standard deviation of 32.0 ± 0.47.
^{††}Analysis of percent platinum content for each replicate resulted in an average ± standard deviation of 33.1 ± 0.24.

Compound 12 (+2NaCl): Compound 3′ was prepared in a similar manner for producing compound 6 (+2NaCl) described above, from Na₂PtCl₄·H₂O (1.20 g, 3.00 mmol), S-methylcysteine (831 mg, 2.05 mol eq) and Milli-Q water (6.0 mL), which afforded 2.04 g.

Compound 13 (+2NaCl): A solution of N-tert-butoxycarbonyl-S-methylcysteine (2.49 g) in anhydrous tetrahydrofuran (26 mL) chilled by ice bath, and then carbonyldiimidazole (3.78 g) was added stepwise. The mixture was stirred in an ice-bath for 4 h, before addition of 28% ammonia solution (6.5 mL) dropwise, followed by stirring an additional 3 h, and storage at 4° C. overnight. The mixture was briefly concentrated by rotary evaporation and extracted with ethyl acetate (100 mL). The extract was washed with 1M HCl (25 mL×2) to remove imidazole. The aqueous phase was re-extracted with ethyl acetate (20 mL×1) and the combined extracts were concentrated by rotary evaporation to generate a solid crude residue that was washed with hexanes/ethyl acetate=2/1 mixture, filtrated, washed with hexanes/ethyl acetate=2/1 mixture, and dried in open-air to afford N-tert-butoxycarbonyl-S-methylcysteine carboxamide (Boc-SMeCysNH₂, 2.49 g). The Boc-SMeCysNH₂ (2.10 g) was dissolved in 1,4-dioxane (9.0 mL) with warming and sonication, before addition of 4 M hydrogen chloride in 1,4-dioxane (9.0 mL, 4.0 eq.). The mixture was kept at the ambient temperature overnight. At this time, the reaction was not complete and more 4 M hydrogen chloride in 1,4-dioxane (4.5 mL, 2.0 eq.) was added. After 4 hours, addition of hexanes (23 mL) promoted precipitation of the product which settled to the bottom of the vessel. The supernatant was removed by decantation and the solid was rinsed with diethyl ether (10 mL×3), with careful decantation each time, and dried in vacuo to afford S-methylcysteine carboxamide hydrochloride (SMeCysNH₂·HCl, 1.53 g). Compound 4′ (+2NaCl) was prepared in an analogous manner for producing compound 6 (+2NaCl) described above, from Na₂PtCl₄·H₂O (401 mg, 1.00 mmol), SMeCysNH₂·HCl (345 mg, 2.05 mol eq) and Milli-Q water (5.0 mL), afforded 631 mg.

### Example 11

### Optimization Studies: Stability & Speciation

Formulation stability of four cyanide scavenging Pt(II) compounds was evaluated. Ligands were selected to enhance the reactivity of the metal center of the Pt(II) compounds by including a methyl thioether group. Each compound was prepared in aqueous media to select for high solubilities and potential hydrolytic stability. During the original discovery efforts for these bidentate (S, N)-ligand Pt(II) compounds, formation of several pH dependent mixed isomers were observed, consistent with prior structural assignments. Summa et al (2006), supra; Norman et al. (1992), supra. However, different isomers of bidentate chelates can alter cyanide binding kinetics. Appleton et al. (1988), supra. As related to formulation stability, the effects of formulation pH can affect in vivo efficacy which warranted further evaluation.

The existing risk of Pt(II)-based therapies to induce adverse side effects can create a dose limiting toxicity, especially when additional mitigation strategies are not utilized. For instance, the prevalence of acute kidney injury (AKI) caused by the chemotherapeutic agent cisplatin alone is high, occurring in about 30-40% of patients that receive the agent. Volarevic et al., Molecular mechanism of cisplatin-induced nephrotoxicity, J Biomedical Sci 26: 25 (2019). Accordingly, it would be beneficial to be able to tune the reactivity of a cyanide scavenger to effectively inactivate the compound and thereby reduce or even prevent toxicity in the patient.

Compounds 6 and 9 were previously shown to be reactive with cyanide but demonstrated different efficacy in lethal zebrafish and mouse cyanide exposure models (see Examples above). In fact, the bis-(L-methionine amide (S,N)platinum(II) dichloride (compound 9) demonstrated a reduction in cyanide reaction at high pH. Behymer et al. (2022), supra. Despite the chemical similarity, compound 6 consistently had higher rates of cyanide scavenging kinetics in comparison to compound 9 across all assays at neutral pH. These observations motivated a more detailed investigation of this pH-dependence on reactivity and the potential for impact on in vivo efficacy.

Platinum complexes are reported to have greater stability with five-membered ring systems which may resist pH-dependent changes. Lawrance, Introduction to Coordination Chemistry, John Wiley & Sons (2013). The enhancement of conformational stability potentially slows the substitution reaction kinetics with cyanide. However, compound 12 demonstrated enhanced rates of cyanide reactivity and in vivo efficacy similar to compound 6 within the resolution of the experimental methods. (See Examples above and Behymer et al. (2022), supra.) Therefore, the potential role of a carboxamide ligand in the five-membered ring system motivated the preparation of compound 13 to serve as a complimentary test case for the effects of a carboxamide group.

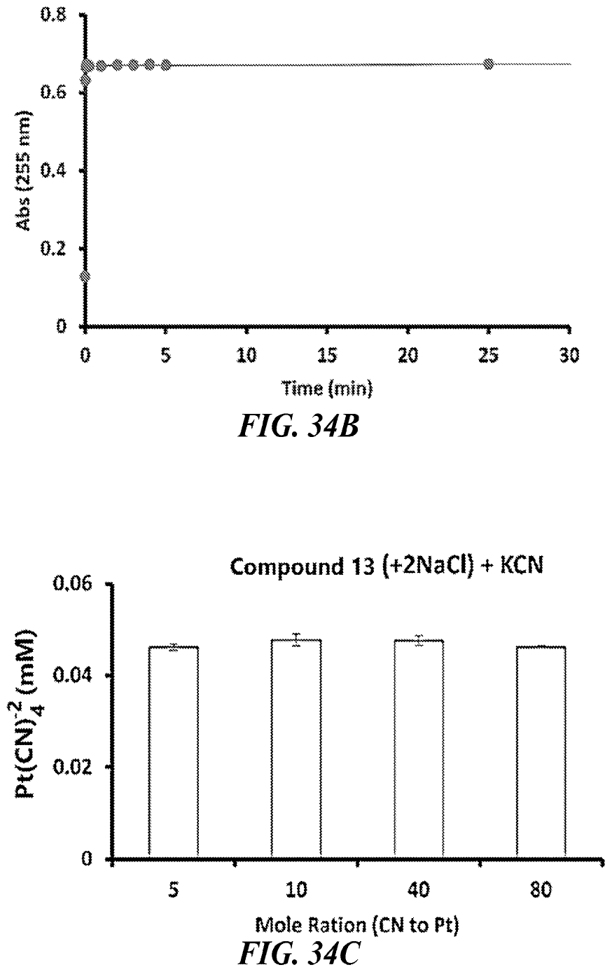

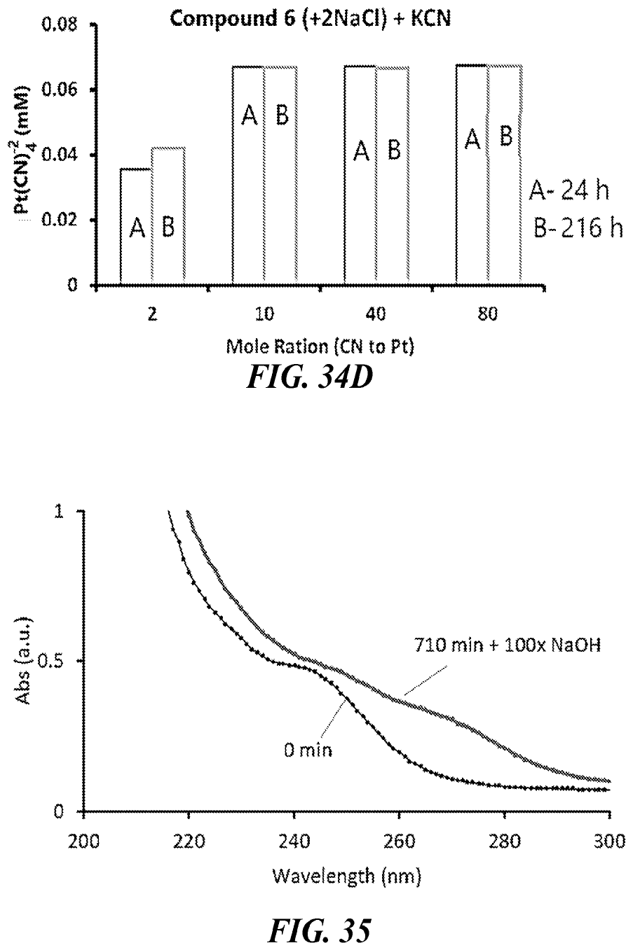

The data in FIGS. 34A-34D was used to obtain an apparent rate constant for Pt(CN)₄⁻² production from compound 13. Further, the results in both FIGS. 34C and 34D demonstrated the signal was stable after 5-10 minutes of cyanide addition.

The initial comparison in UV spectra for compound 13 suggested a pH-dependent change (FIG. 35), similar to those for compound 9. The observed rate constant was estimated as 0.6 min⁻¹ in presence of 40:1 KCN/Pt. Indeed, cyanide reaction rate with compound 13 was observed to be slower than compound 6 (FIG. 34B).

To assess compound stability, the cyanide scavenging of carboxylate complexes (compounds 6, 9, 12 and 13) were each maintained across all pH conditions (e.g., 4-7) at room temperature for up to 14 days and assessed using HPLC. For HPLC, an Agilent 1100 equipped with a DAD to scan wavelengths 200-300 nm, and Restek Ultra IBD 2.1×50 mm was used to detect and separate formulation mixtures of the platinum complexes. The column was operated in reverse phase mode with 5 μL injections at 0.3 mL/min. Baseline conditions were 65% purified water with 2 mM ammonium formate and 35% v/v acetonitrile. Elution phase was carried out by a pH 3.7 solution with 2 mM ammonium formate and 0.5% v/v formic acid, gradient to induce ion exchange. Peak identity of Pt(CN)₄ was quantified using 260 nm and confirmed using absorption spectra from 200-300 nm.

As noted above, stability samples were stored at room temperature for the duration of the 14-day study. HPLC sample preparation was performed by diluting a 10 mM platinum stock to 350 μM, reacting the platinum by adding KCN to a final concentration of 1.4 mM (1:4 Pt to KCN) for 10 minutes at ambient temperature. Reaction solutions were 12.5 mM sodium phosphate pH7.3 buffers. Sample preparations were staggered so each HPLC injection was made at 10 minutes. The 10-minute reaction step was repeated in triplicate for each time point.

For the HPLC speciation assay, an Agilent 1100 equipped with a UV detector (X=220 nm) and Agilent Zorbax Eclipse XD8 C18 column was used with 50 μL injections. Mobile Phase consisted of 88% aqueous:12% acetonitrile. The aqueous component consisted of 25 mM sodium phosphate pH 5.5 with 12.5 mM Heptanesulfonic Acid as an ion pairing agent. The method was performed using an isocratic run for 35 minutes at 1 mL/min.

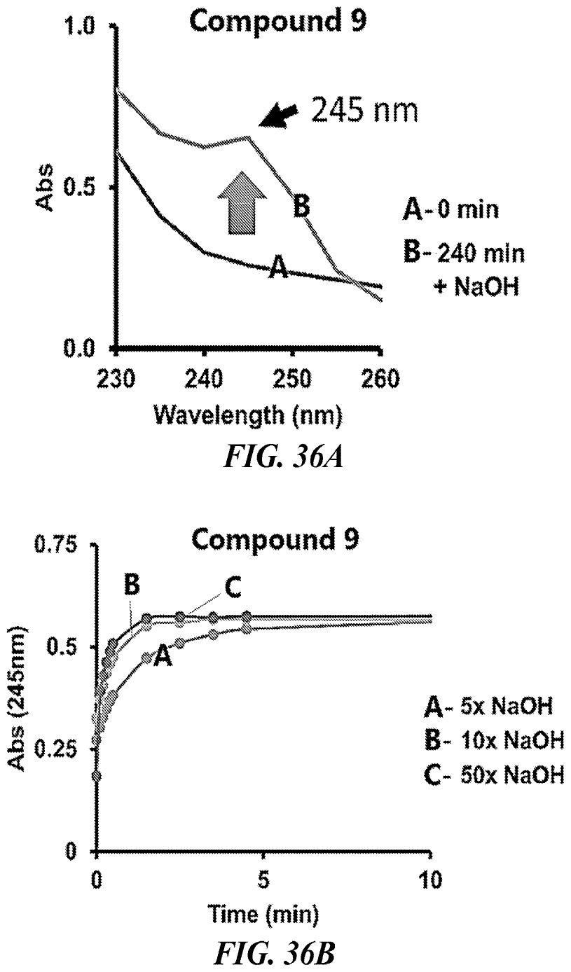

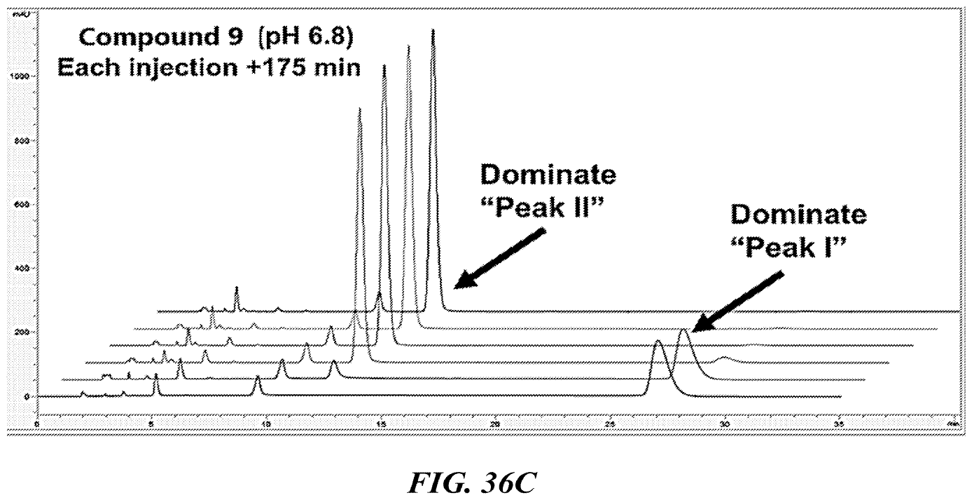

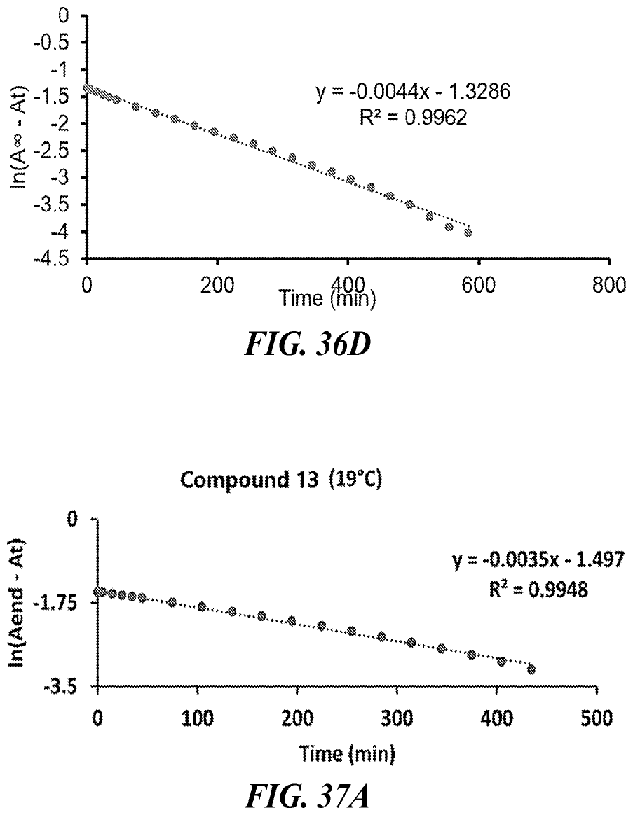

The cyanide scavenging of carboxylate complexes (compounds 6 and 12) was maintained across all pH conditions (e.g., 4-7) at room temperature for up to 14 days. In contrast, the carboxamide compounds (compounds 9 and 13) had reduced formation of Pt(CN)₄²⁻ in the formulations at pH>5 when stored for several days. The instability also resulted in reduced cyanide scavenging activity for the carboxamide compounds 9 and 13. For instance, compound 9 revealed spectral changes in presence of sodium hydroxide (FIG. 36A). In comparison, compounds 6 and 13 are carboxylate complexes, and did not demonstrate the same behavior by UV in presence of NaOH. A pH-dependent rate is demonstrated for compound 9 in FIG. 36B, which suggests the potential for a base-catalyzed process.

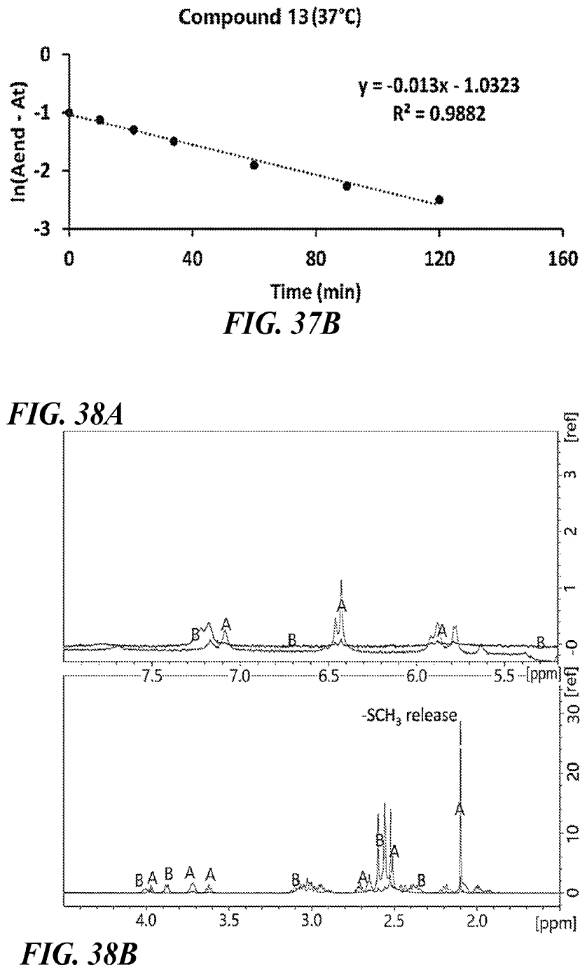

Evaluation by UV at 245 nm for the conversion rate of compound 9 at 18.8° C., pH 7.3 was found to have a half-life of 2.6 hours (FIG. 36D), while compound 13 had an apparent half-life of 3.3 hours and 9 minutes at 19° C. and 37° C., respectively (FIGS. 37A and 37B).

HPLC analyses of compound 9 in pH 6.8 phosphate buffer at room temperature shows gradual changes over 12 hours when injected repeatedly, likely due to a speciation event in FIG. 36C. Initially there was a dominate form, labeled as Peak I at about 33 minutes (Form 1) which disappeared after 4 hours giving rise to a new Form 2 labeled as Peak II (FIGS. 36A-36C).

The UV and HPLC observations were in general agreement with NMR. In FIG. 38A, the ¹H NMR signals at 7.25 and 7.18 ppm reduced and shifted upfield when day 0 (labeled B) and 3 (labeled A) were compared. In addition, new signals between 5.5 and 6 ppm emerged on day 3. FIG. 38B also demonstrates a change that occurred simultaneously. The singlet signal at 2.1 ppm is assigned as —SMe signal signifying that the functional group was unbound to platinum, where a significant increase in intensity was observed on day 3. These data suggest compound 9 lost interaction between thioether and platinum. The loss of thiomethyl binding to platinum is most visible in the signal at 2.1 ppm (consistent with the expected chemical shift for free methionine), and grows much more dominant at day 3, while the two larger signals between 2.6 ppm and 2.55 ppm (day 0, labeled B) for bound methyl groups shifted to 2.5 ppm and became less intense.

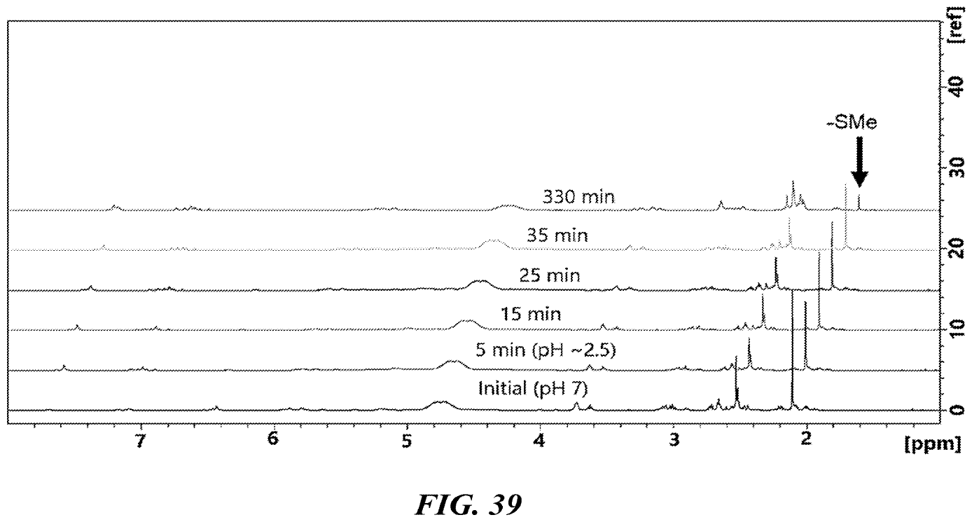

Analysis of the reversibility for compound 9 in pH 7 for 3 weeks was performed by ¹H NMR (FIG. 39). Compound 9 was incubated in pH 7 phosphate buffer for 3 weeks (labeled B in FIG. 39) and HCl was added to adjust the solution pH to approximately 2.5. The addition of HCl to reduce the pH to approximately 2.5 showed a gradual loss of free —SMe signal at 2.1 ppm, which supports reassociation of —SMe to with platinum.

The functional stability of the platinum complex formulations is described as their ability to retain reactivity with cyanide during storage in solution. Platinum-thioether compound 9, which yielded slower reaction rates with cyanide, particularly when stored for several days, was less efficacious in fish and mouse models. (See Examples above and Behymer et al. (2022), supra.)

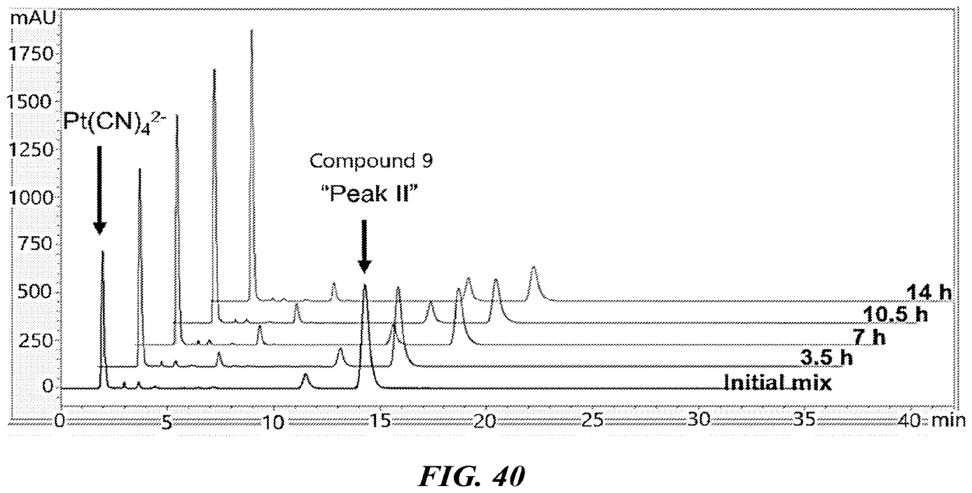

To further assess this, compound 9 (2 mM) was incubated in pH 6.8 phosphate buffer for 3 days at room temperature. Upon the addition of 4 mole equivalences of cyanide, Form I as shown in FIG. 40 immediately disappeared while a slower conversion of Form II occurred over several hours. Pseudo-first order conditions of compound 9 with cyanide at a 1:40 molar ratio in pH 7 phosphate shows a rate of 0.32 min⁻¹. Under similar conditions, when compound 9 was freshly prepared, the observed rate with cyanide was >15 min⁻¹. (See Examples above and Behymer et al. (2022), supra.) Thus, the rate of cyanide scavenging for compound 9 stored at a neutral pH is significantly slower than a freshly prepared solution.

Under a similar set of conditions, the reaction rate of compound 13 with cyanide was also diminished (FIG. 36D).

Accordingly, for compounds 9 and 13, the reactivity loss was observed when the pH of the solution approached neutrality, implicating a role for the carboxamide groups distinct from the carboxylate groups in compounds 6 and 12. To substantiate the apparent differences in reactivity at different pH values, the platinum complexes were stored in a range of buffers for up to 14 days. At different time points, samples of the formulations were reacted with 4 mole equivalences of KCN for 10 minutes and product (Pt(CN)₄²⁻) was quantified by HPLC (n=3 for each pH condition).

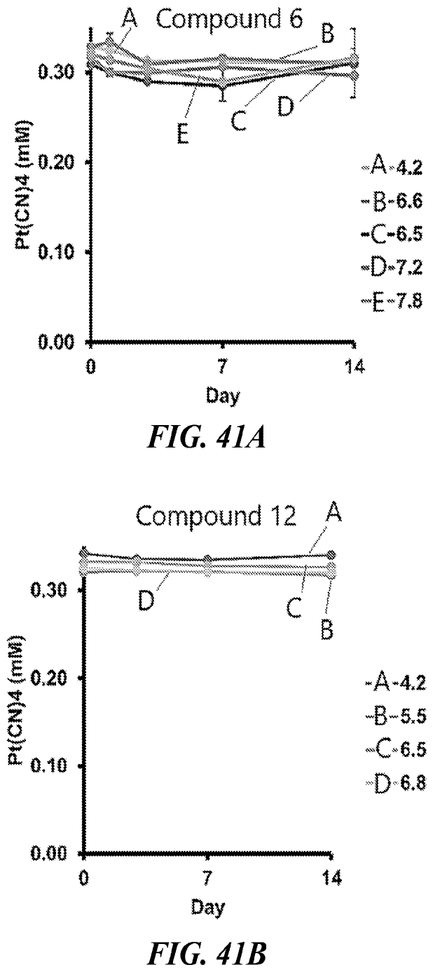

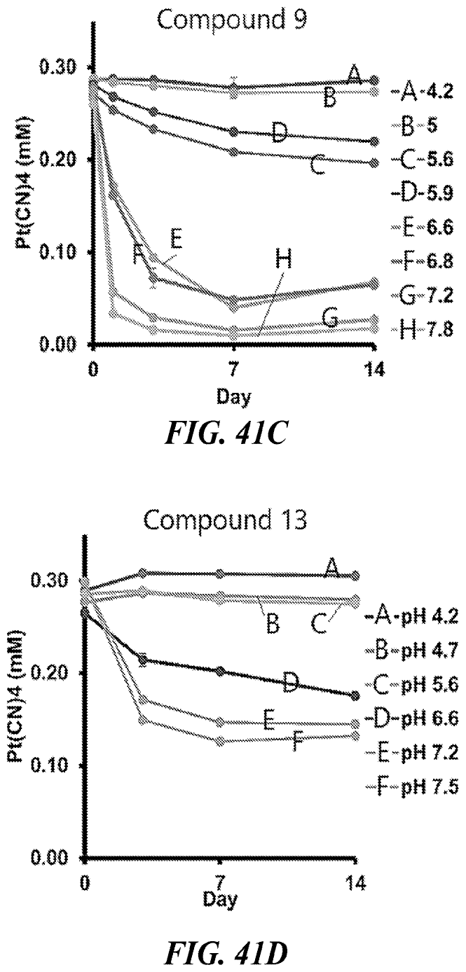

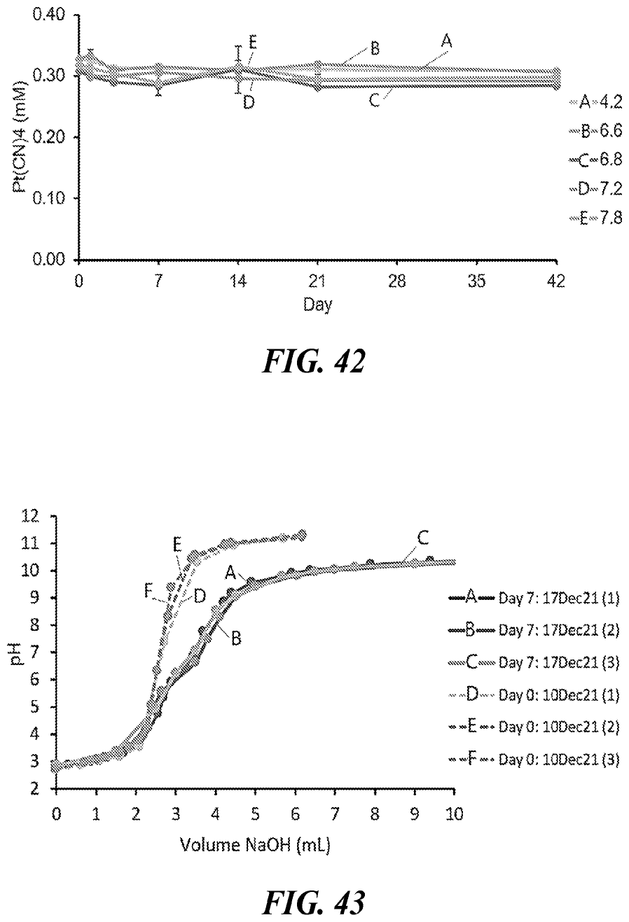

In all pH conditions, compounds 6 and 12 did not show significant changes in reactivity as evident by Pt(CN)₄²⁻ production (FIGS. 41A and 41B) over the 14-day period. Monitoring compound 6 for up to 42 days (FIG. 42) suggests a potential stability much greater than 14 days. As anticipated, the carboxamide complexes 9 and 14 generated reduced amounts of Pt(CN)₄²⁻ over the 14 days at pH values≥5 (FIGS. 41C and 41D).

The observed reduction in cyanide-dependent conversion to Pt(CN)₄²⁻ for compounds 9 and 13 upon storage in pH ranges 5-8 in FIGS. 41C AND 41D are consistent with the results observed in FIGS. 36A-36C, 38A, 38B, and 40. The pH dependent changes in forms of compounds 9 and 13 were revealed by monitoring cyanide reactions, where biphasic kinetics were observed leading to significantly slower production of Pt(CN)₄²⁻ from the second species formed. The data are consistent with the modulation of cyanide scavenging rates of compounds 9 and 13 being the result of a pH-induced isomerization of the complexes to a second form.

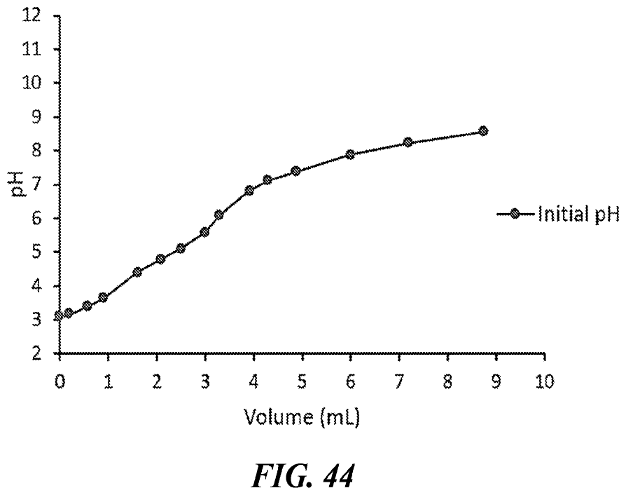

Upon reconstitution of the solid preparations for compounds 6 and 9 in water, an acidic solution was formed. Initial acid-base titration of compound 6 (FIG. 43) reveals an apparent pKa at 2.9. At the equivalence point, approximately two moles of NaOH per Pt(II) is consistent with a carboxylate neutralization. However, when the acidic solutions of compound 6 were stored for an additional 7 days at room temperature before titration with NaOH, a very different profile was observed (FIG. 44). The extended range of NaOH needed to produce incremental increases in pH was indicative of a complex process. The titration of compound 9 was also complex and did not show obvious equivalence points (FIG. 44). Furthermore, leaving compound 9 in water for several days did result in a slow acidification of the pH over time after each titration step, which could be due to the complex undergoing a transition to a new form.

The observed pH dependence differences in stabilities between carboxylates (compounds 6 and 12) and carboxamide ligands (compounds 9 and 13) support a mechanism of intramolecular isomerization, possibly via amine deprotonation as a critical step to facilitate ring closure. Pt-ligand bond angles in a 5-membered ring should have greater stability than 6-membered structures based on the Pt—S/N bond lengths being shorter. Lawrance (2013), supra. As indicated As indicated by UV and HPLC, the results indicate the rate of a new species formation is slower for compound 13 than compound 9. In the prevent investigators' previous study, compound 12 showed slower cyanide scavenging rates than compound 6 reflecting higher conformational stability resulting in slower substitution kinetics with cyanide. A reduced cyanide scavenging rate was also observed under the same conditions for compound 13. These observations support that the S,N-chelate size appears to influence isomerization and cyanide scavenging rates, with 5-member ring structures being more stable than 6-member structures bound to Pt(II).

### Example 12

### Efficacy of Formulations In Vivo

The formulation pH conditions for intramuscular administration were screened to identify optimal conditions to maintain cyanide scavenging for each Pt(II) complex. Compounds 6, 9, 12, and 13 were first tested in the lethal cyanide challenged zebrafish survival assay to confirm stability for in vivo scavenging activity over several days. Nath et al. (2017), supra. Each of the formulations were prepared in buffers from pH 4.3-7.6, and the details for each formulation are summarized in Table 9. Several buffer conditions were prepared for each compound (6, 9, 12, and 13) at approximately 10× the platinum concentration to ensure stable pH during shipment. Each sample efficacy was tested after 5 days for compounds 6 and 9, and after 4 days for compounds 12 and 13 to confirm activity.

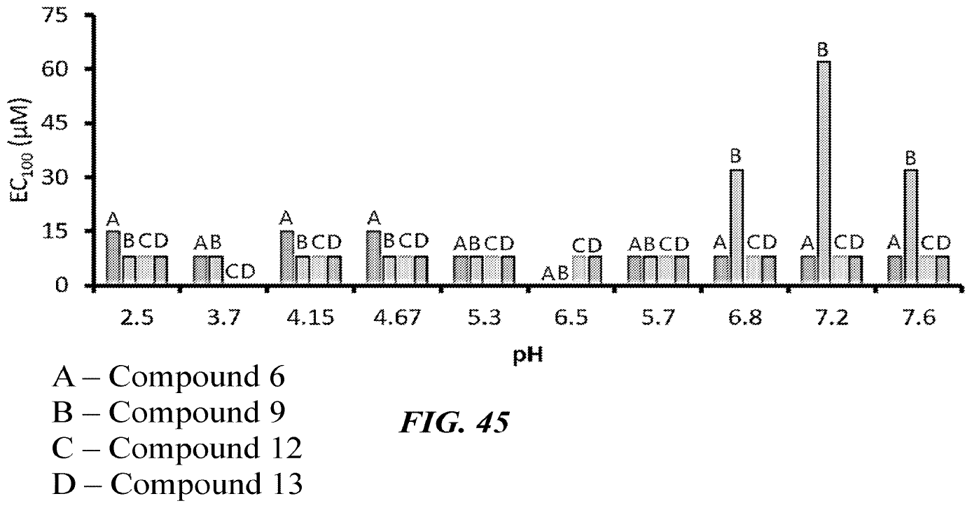

The results in FIG. 45 demonstrate that compounds 6, 12, and 13 remained efficacious (EC₁₀₀≤15 μM) across all pH values. Accordingly, all four compounds can operate in optimal pH ranges to react with a 1:4 stoichiometric formation of Pt(II):CN. Compound 9 had decreased efficacy at pH 6.8 and higher, suggesting that the speciation has a significant effect on in vivo rescue.

The zebrafish efficacy results were consistent with the in vitro findings for compound 9, as slower cyanide scavenging kinetics occurred when compound 9 was stored near neutral pH values. Together, these data suggest a lower pH should be considered for formulations stored for several days.

TABLE 9
Zebrafish formulation details.

| Buffer | pH of Formulation* |
|---|---|
| Acetate | 3.77 |
| Acetate | 4.2 |
| Citrate | 4.8 |
| Citrate | 6.0 |
| MES | 5.5 |
| Phosphate | 7.0 |
| HEPES | 7.00 |
| Tris | 7.20 |
| Bis-Tris | 6.5 |
| Water | ~2.5 |

The formulations of compounds 6, 9, 12, and 13 were also tested for efficacy in a previously described lethal cyanide inhalation mouse model via intramuscular injection. (See Examples above and Behymer et al. (2022), supra, and Chan et al., The combination of Cobinamide and Sulfanegen is highly effective in mouse models of cyanide poisoning, Clin Toxicology (Phila) 49(5): DOI: 10.3109/15563650.2011.584879 (2011).)

A primary motivation was to identify a suitable formulation of compounds 6, 9, 12, and 13 that retained efficacy by IM injection. Prior studies were conducted with compounds 1-3 and prepared as +2NaCl forms (α) (described above in Example 7). To assess the impact of osmolality on IM administration an alternative procedure to reduce NaCl (β) was investigated.

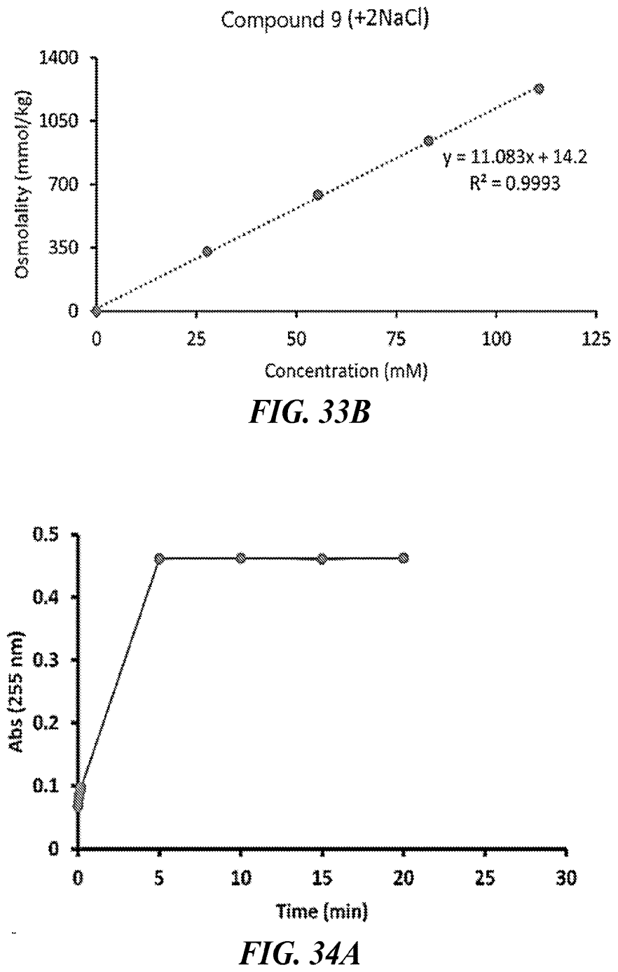

Briefly, the osmolality of each sample was recorded using a vapor pressure using a VAPRO® Vapor Pressure Osmometer Model 5600 with 10 μL sample volume. An example calibration and analysis for preparations of compounds 6 and 9 are shown in FIGS. 33A and 33B. A linear decrease as a function of concentration was observed, and osmolality decreased linearly with the concentration with a correlation coefficient>0.99.

Compounds prepared as either +2NaCl form (α) or no NaCl form (β) were compared by testing in the mouse lethal cyanide inhalation model using IM administrations. Previously, compound 6α was found to be 100% efficacious at 87 μmoles/kg. (Example 7; Behymer et al. (2022), supra). In addition, the efficacy of compound 9α was only efficacious for 3 out of the 5 mice at 87 μmole/kg. Id.

In Table 10, compound 6β also shows 100% survival when dosed as low as 72 μmole/kg in the lethal mouse model. A fresh preparation of compound 9β at pH 6.5 was able to provide survival in all 4 mice that were treated at 103 μmole/kg and pH 4.2 shows complete rescue at 87 μmole/kg. These results suggest lower pH provides a stable formulation.

It was hypothesized that with the 5-membered ring in compound 13a might reduce the rate of speciation due to optimized ring strain, improving the stability of the formulation. The results for compound 13a revealed total rescue at 103 μmole/kg, as indicated in Table 10. The results from the lethal cyanide treated mouse model demonstrate that low pH can improve the efficacy of intramuscularly delivered platinum compounds 9 and 13 with the carboxamide ligand by reducing speciation. Furthermore, preparations with reduced NaCl content and reduced osmotic pressure also retain efficacy.

TABLE 10
Mouse efficacy for platinum compounds 6, 9, 12, and 13.

| Compound | $\frac{\begin{matrix}Dose \\ µ mole Pt\end{matrix}}{kg}$ | $\frac{\begin{matrix}Dose \\ mg Pt\end{matrix}}{kg}$ | pH | Survival | Osmolality (mmol/kg) | Reference |
|---|---|---|---|---|---|---|
| 6 β | 51 | 10 | 6.5 | 3/4* | 153 | |
| 6 β | 72 | 14 | 6.5 | 4/4 | 153 | |
| 6 α | 87 | 17 | 6.5 | 4/4 | 565** | Behymer et al. |
| | | | | | | (2022), supra. |
| 9 β* | 103 | 20 | 6 | 4/4 | 346 | |
| 9 β | 103 | 20 | 6.5 | 1/3 | 611 | |
| 9 β | 56 | 11 | 4.2 | 3/4 | 210 | |
| 9 β | 87 | 17 | 4.2 | 4/4 | 230 | |
| 9 β | 103 | 20 | 4.2 | 4/4 | 618 | |
| 12 α | 97 | 19 | 6.5 | 5/5 | 608** | Behymer et al. |
| | | | | | | (2022), supra. |
| 13 α | 103 | 20 | 4.2 | 4/4 | 625 | |

*Compound 9 was reconstituted and immediately injected intramuscularly.
** Osmolality was estimated using the osmolality concentration curve for compound 6 herein and assuming 150 mmol/kg as the average phosphate buffer contribution. Compounds prepared as +2NaCl form (α) or no NaCl form (β).

At least in part, the findings reveal that compounds 6 and 12 maintain similar kinetic activity towards cyanide across the pH ranges studied. However, compounds 9 and 13 have reduced cyanide scavenging kinetics when formulated above pH 5 after three days. Compounds 9 and 13 were stable and maintained reactivity at pH ranges≤5. Optimal formulation pH conditions were determined for all four compounds and led to the rapid stoichiometric formation of Pt(CN)₄²⁻ exceeding the binding of any known scavenger approved or in development.

### Example 13

### Nephrotoxicity of Platinum Formulations

A rat model was used to evaluate changes in blood chemistry and complete blood counts in a dose-dependent manner to assess tolerability of a single intraperitoneal (IP) injection of compounds 6, 9, 12, and 13. Garrett & Korstanje, Using genetic and species diversity to tackle kidney disease, Trends in Genetics 36: 499-509 (2020); Kohl et al., Evaluation of urinary biomarkers for early detection of acute kidney injury in a rat nephropathy model, J Pharmacological & Toxicological Methods 105: 106901 (2020).

Briefly, Sprague-Dawley rats (Envigo and Inotiv) with weight ranges of 225-250 grams were used for this study performed at the Purdue Translational Pharmacology and Clinical Veterinary Pathology Laboratories. Compounds 6 and 12 were formulated in phosphate buffer. The final pH of the formulation at the time of injection was 6.5-7.0 using sodium hydroxide for final pH adjustment. Compounds 9 and 13 were prepared in sodium acetate buffer (Ca2⁺- and Mg2⁺-free) with final pH adjustments using sodium hydroxide to a target pH of 4.2. Final adjustments were made using MilliQ water to reduce osmolality of the solution.

Platinum and vehicle were administered by intraperitoneal (IP) injection in ≤5.28 ml/kg. Doses were scaled allometrically using body surface area differences between mice and rats and designed to reach five-times the efficacious dose in the lethal cyanide mouse model but using TP administration (Table 11).

TABLE 11
Rat toxicity formulation details for compounds
6, 9, 12, and 13.

| | | | Mouse | | |
|---|---|---|---|---|---|
| | | | Equi- | | |
| Com- pound | $Dose \frac{μmole Pt}{kg}$ | $Dose ( \frac{mg Pt}{kg} )$ | valent Dose | pH | Osmolality (mmol/kg) |
| 6 α | 44 | 8.5 | 1x | 6.5 | 1300 |
| 6 α | 109 | 21.3 | 2.5x | 6.5 | 1060 |
| 6 α | 145 | 28.3 | 3.3x | 6.5 | 1100 |
| 6 α | 218 | 42.5 | 5x | 6.5 | 1250 |
| 6 α | 218 | 42.5 | 5x | 6.5 | 487 |
| 9 α | 218 | 42.5 | 5x | 4.3 | 600 |
| 9 α | 218 | 42.5 | 5x | 6.5 | 607 |
| 12 α | 223 | 43.4 | 5x | 6.6 | |
| 13 α | 218 | 42.5 | 5x | 4.3 | 625 |

Compounds prepared as +2NaCl form (α) or no NaCl form (β).

Blood samples were drawn 1- and 5-days post-injection and processed for Comprehensive Metabolite Panel and Complete Blood Count Panel. At the end of the study, the animals were euthanized following the PHS Policy on the Human Care and Use of Animals, Guide for the Use and Care of Laboratory Animals. All methods were carried out in accordance with the regulations and guidelines of the Animal Welfare Act and the American Association for Accreditation of Laboratory Animal Care. The IACUC committee at Purdue University approved all experimental protocols (1405001069).

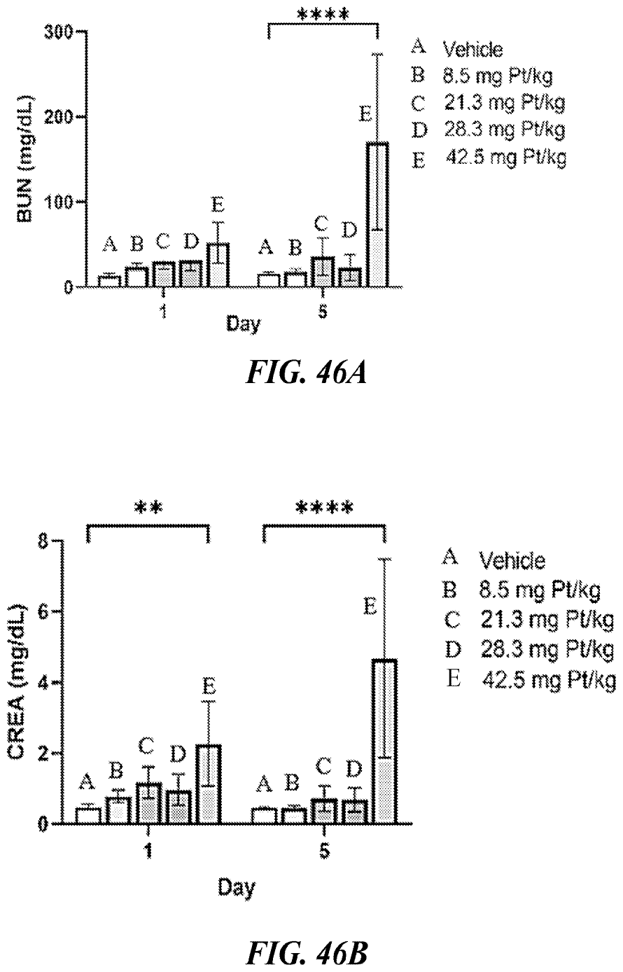

Animals treated with compound 6 up to five-times (218 μmole/kg) the efficacious dose exhibited a significant increase in both blood urea nitrogen (BUN), creatinine (CREA), and phosphate (PHOS), indicating acute kidney injury (AKI) (FIGS. 46A and 46B). Briefly, each animal received compound 6α in pH 6.5 phosphate buffer with an injection volume of ≤2.12 ml/kg. Each group 44-218 μmole/kg (8.5-28.3 mg Pt/kg) had n=6 rats, vehicle, and 42.5 mg P/kg containing n=12 rats.

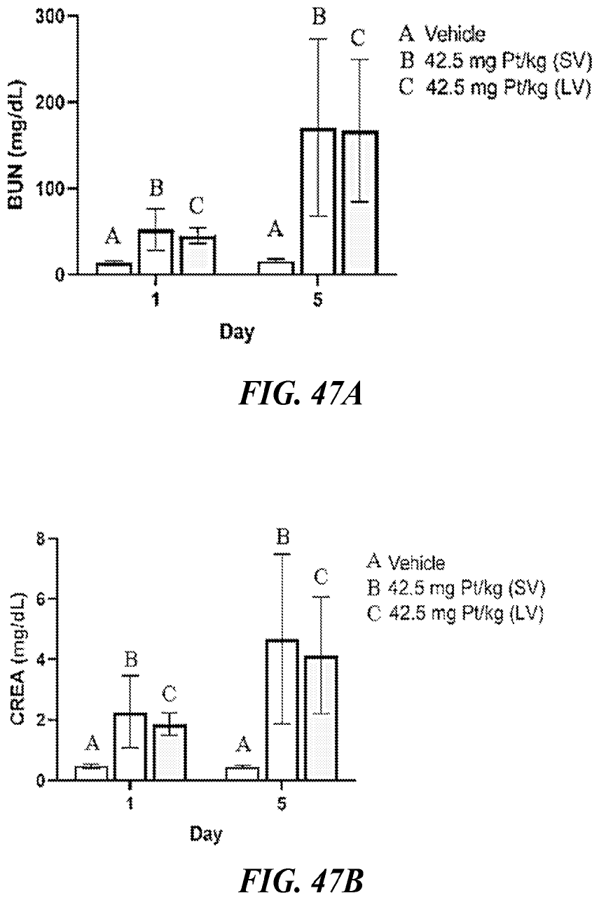

In addition, the compound 6p form with reduced NaCl in the formulation was used to assess the potential impact of osmotic pressure. Compound 6 was administered in a small volume (SV) 2.2 ml/kg to a first cohort (n=11), and a large volume (LV) 5.28 ml/kg to a second cohort (n=6). Analysis was carried out using an ordinary two-way ANOVA with a Ŝidák multiple comparisons comparing the cell means regardless of rows and columns. No significant difference was observed in the AKI markers between animals treated with compound 6α or compound 6β at the highest doses, thus reducing a role of osmotic pressure (FIGS. 47A and 47B).

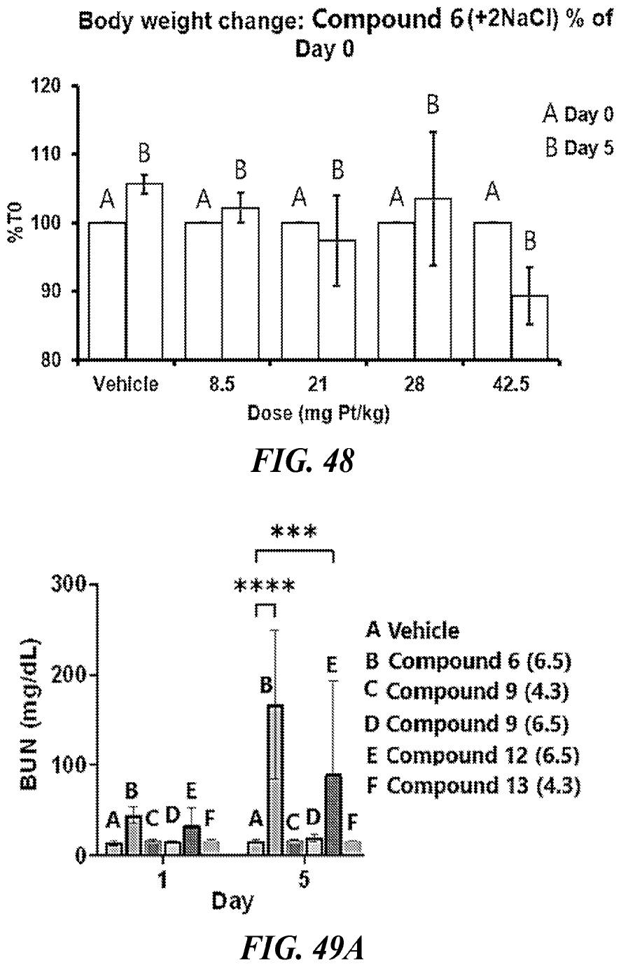

Additionally, to assess body weight change and, thus, toxic stress, animals received compound 6 in pH 6.5 phosphate buffer with an injection volume of ≤2.2 ml/kg. Each group 8.5-28.3 mg Pt/kg had n=6 rats, a vehicle, and the group of 42.5 mg P/kg rats contained n=12 rats. The animals lost approximately 15% body weight when dosed with the highest amounts of compound 6α or compound 6β after 5 days, consistent with some level of toxic stress caused by a Pt(II) species (FIG. 48). Compound 12 also demonstrated significant increases in BUN, CREA, and PHOS levels after 5 days with the five-times (223 μmole/kg) efficacious dose. Although the average marker levels appeared to be lower than compound 6 at similar doses, these results were not significantly different.

As shown in FIGS. 49A-49C, the carboxamide containing ligand compounds 9 and 13 exhibited distinctly different impact on the blood chemistry and complete blood counts. Compound 9 was tested in both pH 4.3 and 6.5 formulations at the five-times (218 μmole/kg) the mouse efficacious dose. No significant differences in AKI markers were observed with either formulation of compound 9 (9α and 9β) when compared to the control group.

Rats treated with compound 13 at pH 4.3 and 218 μmole/kg had BUN, CREA and PHOS levels similar to the control group, and significantly lower than those dosed with compound 12. Thus, both compounds 9 and 13 have a significantly reduced risk of inducing AKI at five-times the efficacious dose when contrasted to their carboxylate counterparts, compounds 6 and 12.

The data supports that the free carboxylates of each amino acid yield fast cyanide scavenging Pt(II) compounds 6 and 12, but can also increase AKI at higher doses that can lower the therapeutic index. Alternatively, carboxamides on the bidentate amino acid ligands of the compound (e.g., compounds 9 and 13) have reduced scavenging kinetics when formulated at pH>5; however, an optimized pH formulation of the carboxamide compounds not only maintained cyanide reactivity, but also resulted in a significant reduction in the risks of platinum-induced toxicity (i.e., AKI).

While the study was not designed with the proper power to assess significant gender differences, females did appear to be less prone to increases in the AKI markers from the Pt(II) compounds and 12 than males (FIGS. 50A and 50B). There is substantial data from animal model studies that highlight gender differences associated with cisplatin-induced toxicities. Marcu, Gender and sex-related differences in normal tissue effects induced by platinum compounds, Pharmaceuticals 15: 255 (2022). Finally, complete blood count values did not have any meaningful differences between complexes and controls, and thus the results were not shown.

Cyanide exposure is difficult to detect in patients and can lead to concerns depending upon the safety of therapeutic intervention. As such, evaluating the risks of toxicities associated with platinum complexes (e.g., AKI) is critical. Sprague-Dawley rats are well-recognized as a sensitive model for detecting platinum-induced nephrotoxicity. Perše & Veĉerić, Cisplatin-induced rodent model of kidney injury: characteristics and challenges, Biomedical Research Int'l 2018: 1462802 (2018). At doses five times the efficacious dose in mice, compounds 6 and 12 resulted in symptoms for AKI after five days consistent with observations in cisplatin-treated Sprague-Dawley rats. Brenner et al., Comparison of cobinamide to hydroxocobalamin in reversing cyanide physiologic effects in rabbits using diffuse optical spectroscopy monitoring, J Biomedical Optics 15(1): 017001 (2010). Interestingly, carboxamides in compounds 9 and 13 significantly alleviate the observed AKI in the rat model. While a correlation between Pt reactivity and toxicity has been discussed previously, this study shows that platinum complexes can slowly convert to less toxic forms to create agents with improved safety.

Ligands such as methionine with a thioether functional group have been used to ameliorate the nephrotoxic effects of cisplatin in rats. Jones et al. (1989), supra; Jones et al. (1991), supra; Basinger et al (1990), supra. Thioethers and other strongly binding ligands to platinum have been proposed to function as antioxidants. Stankovic et al (2020), supra. For instance, sulfhydryl supplied by glutathione or anion sulfur ions (e.g., thiolate and WR-2721) have been used to mitigate cisplatin-induced nephrotoxicity; however, platinum anticancer agents' efficacy is compromised in these cases. Jones et al. (1991), supra. The most promising antioxidants such as dithiocarbamates and WR-2721 show decreased renal platinum levels. Jones et al., Relative effectiveness of some compounds for the control of cisplatin-induced nephrotoxicity, Toxicology 68: 227-247 (1991); Volarevic et al (2019), supra. For the acute cyanide scavenging setting, using rapid-acting platinum rescue agents offers benefits as a single bolus IM dose. These comparisons of compounds 6, 9, 12, and 13 afford insights into the potential balance needed to achieve the safety and efficacy of S,N-chelated Pt(II) complexes with a therapeutic index of 5.

### Example 14

### Pharmacokinetics of Intramuscular Formulation

A proof of concept study was performed to assess the similarities and potential differences in the pharmacokinetic parameters between compounds 6 and 9 in rats. The data obtained will be used to estimate the doses needed upon allometric scaling to achieve efficacy.

The study design involved two groups of 5 rats (2 male and 3 female), each treated with 45 μmole/kg of either compounds 6 or 9. Each compound was administered IM to mimic the efficacy model and product concept. Plasma levels of total Pt in animals were monitored for 48 hours (FIG. 51), and 250 μL of blood was sampled at each time interval and replaced with phosphate buffered saline using Culex™ stress free autosampler units. Samples were collected at 0, 5 minutes, 10 minutes, 20 minutes, 30 minutes, 45 minutes, 2 hours, 4 hours, 8 hours, 12 hours, 24 hours, and 48 hours post administration. At the time of sample collections, blood samples were immediately centrifuged, and the resultant plasma was stored in separate vials for each time point at −80° C.

These data were evaluated for differences in absorption rates, elimination rates, and overall exposure by AUC values (Table 12). Both complexes appeared to reach maximal concentrations within 7-9 minutes after IM administration, which is consistent with a highly diffusive agent that is capable of rapid absorption. However, significant differences between the dose normalized maximal concentration

$$( \frac{{C}_{max}}{dose} )$$

in plasma for compound 6 was about 2.2 times greater than that for compound 9.

TABLE 12
Pharmacokinetic parameters acquired in
rat PK studies for compounds 6 and 9.

| Parameter | Compound 6 | Compound 9 | p-value |
|---|---|---|---|
| Cₘₐₓ/Dose | 16805 ± 2196 | 7685 ± 3245 | >0.005 |
| Tₘₐₓ (hr) | 0.150 ± 0.109 | 0.117 ± 0.0456 | 0.547 |
| MRT (hr) | 59.2 ± 30.9 | 39.4 ± 10.5 | 0.213 |
| Elim. T_{1/2} (hr) | 43.9 ± 20.9 | 27.8 ± 8.48 | 0.148 |
| AUC₀₋₄₈ (g*hr/L) | 0.203 ± 0.0329 | 0.271 ± 0.104 | 0.203 |
| AUC_{0-∞} (g*hr/L) | 0.361 ± 0.0977 | 0.386 ± 0.128 | 0.736 |
| AUC_{0-∞}/Dose (g*hr/L) | 169415 ± 45866 | 151988 ± 50456 | 0.583 |
| CL (mL/h/kg) | 6.21 ± 1.47 | 7.28 ± 2.70 | 0.463 |

P-values reported were obtained using the two-tail distribution and values were determined using n = 5, 3 females and 2 males. Data shown is an average ± standard deviation.

The FIG. 51 inset highlights a biphasic disposition profile observed for compound 6, which suggests a dominant distribution phase in the first two hours. In contrast, compound 9 appeared to have a steady disposition phase, where the distribution phenomena were not kinetically different from the elimination rate. Despite these possible differences, the AUC and clearance rates revealed a lack of statistically significant differences between the two compounds. The extrapolated clearance rates and the terminal elimination half-lives for compounds 6 and 9 were also not statistically different. These elimination rates appeared faster than those reported for other platinum drug complexes in rats. Wang et al., Pharmacokinetics and tissue distribution of novel traditional Chinese medicine-platinum anticancer agents in rats, J Inorganic Biochemistry 101: 909-917 (2007). Together, these results highlight that compounds 6 and 9 have utility as cyanide scavenger agents with rapid absorption by IM administration, but also indicate different disposition outcomes despite the relatively small structural differences.

### Example 15

### Blood-Brain Barrier (BBB) Permeability Study

Cyanide can be a potent neurotoxin as well, thus distribution of compounds 6 or 9 into the brain may help to reveal the potential to mitigate neurotoxicity. To evaluate the potential for brain distribution, the permeability of compounds 6 and 9 was assessed across an in vitro blood brain barrier (BBB) triculture model as shown in Table 13 and described in Lubin & Knipp, Design of experiment based optimization of an in vitro direct contact triculture blood brain barrier model for permeability screening, Pharmacy & Pharmacology Int'l J 9: DOI: 10.15406/ppij.2021.09.00340 (2021).

Briefly, permeability rates were tested in the apical (A; blood facing) to basolateral (B; neuronal side) direction and in the basolateral to apical (B to A) direction to determine the relative brain parenchymal exposure and efflux ratio (P_{app,B→A}/P_{app,A→B}) for compound 6 (Met₂PtCl₂ (+2NaCl)) and compound 9 (Met(NH₂)₂PtCl₂ (+2NaCl)). For each permeability coefficient determination, 100 μM of each platinum complex was dissolved in Ca²⁺ and Mn²⁺ containing Hanks' Balanced Salt Solution (HBSS). The osmolality of the sample solutions were between 240-260 mmol/kg. The efflux ratio for compounds 6 and 9 were 1.10 and 0.98, respectively, supporting that the permeation rates are controlled by passive diffusion.

Permeation rates were determined by loading complex on the apical side for A-B permeability and basolateral for B-A permeability. The receiver chamber had 100 μL sample aliquots taken at 0, 15, 30, 45, 60, 120, and 180 minutes and analyzed by HPLC. Analysis was carried out at 220 nm using an Agilent Zorbax Eclipse XD8 C18 column.

TABLE 13
Apparent permeability (Pₐₚₚ) of compounds 6 and 9 across
a direct contact BBB triculture model. Results are
shown in the average ± deviation of 3 replicates.

| | A-B Permeability | B-A Permeability |
|---|---|---|
| | (×10⁻⁵ cm/s) | (×10⁻⁵ cm/s) |
| Compound 6 | 1.95 ± 0.27 | 2.15 ± 1.28 |
| Compound 9 | 1.84 ± 0.19 | 1.81 ± 0.04 |

The permeability coefficients compare favorability with rates determined for established markers having higher in vivo brain distribution and permeation across the BBB. Kulczar et al., Development of a direct contact astrocyte-human cerebral microvessel endothelial cells blood-brain barrier coculture model, J Pharmacy & Pharmacology 69: 1684-1696 (2017). The BBB permeation rates support that both complexes can scavenge cyanide in the brain parenchyma.

### Example 16

### Formulation: Tunable Ligands

The down-selection process for Pt(II) compounds/agents and their formulations involves testing the established in vivo high-throughput model of cyanide poisoning. Among the agents identified in Table 1 above, using the thiosulfate ligand appears detrimental to the agents' utility despite being recommended for offsetting cisplatin toxicity. Curiously, thiosulfate is also a known scavenger of cyanide.

The agents of Table 1 are options for use as a ligand in connection with one or more compounds hereof None of the actives in Table 1 have presented lethality to the zebrafish when tested up to 250 μM (5× higher than cyanide) and, in addition, they exhibited increased potency as compared to HCP-AKN and cisplatin-AKN. The compounds in Table 1 (except for the thiosulfate ligand) represent candidates for resynthesis and retesting before advancing to renal toxicity testing in porcine kidney cells and zebrafish studies.

The process for formulating Pt(II) agents starts from either PtCl₂ or Na₂PtCl₄H₂O. Each offers different advantages regarding the final product's elemental composition and purity. The initial reaction conditions requires solubilization of the Pt in water by reaction with the bidentate ligands (such as those or similar bidentate ligands described herein). Water solubility criteria targets 200 mM or 50 mg/mL to provide ample concentrations to meet the requirements of autoinjectors in the field use for IM administration. ligand and Pt ratios are carefully selected to enable isolation of the mono-addition product or the bis-addition products. The preparation of mixed ligand materials such as MetPt(taurine)₂ (Table 1) was accomplished by isolation of an intermediate mono-addition product (MetPtCl₂) before reacting with a second orthogonal ligand (taurine). Additional methods are known for isolating materials that vary in salts, depending upon the starting materials and isolations.

Ligand design principles has been guided in the past by use of methyl sulfide linked through either two or three carbons to a primary amine group. Synthesis of the present ligands aligns with this effort. For instance, a dimer of methionine bridged through the S-methyl groups was prepared (identified as (bridged-Met₂)Pt in Table 1). The new ligand variants include, without limitation, carboxamide variants of the carboxylates, which change the molecule's titratable groups; alterations of the electron donating or withdrawing characteristics at the S-Me group; and the flexibility of the carbon chain bridging the sulfur and nitrogen atoms. Combinations of different ligands on the Pt(II) center is pursued to incorporate agents such as taurine (reported to have renal sparring activity).

Elemental compositions are determined by a combination of ICP-MS and NMR. A solution of known weight per volume is prepared. This is analyzed by ICP-MS to determine platinum content and by NMR of various nuclei (H, ¹³C, ³⁵Cl, ²³Na) for ligand and salt content. All quantitation is performed by comparison to known standard samples. The reproducibility of the batch productions will be a significant factor in these studies.

Methods have been developed to assess the cyanide reactivity and stoichiometry of each Pt(II) complex. The reaction of platinum complexes with cyanide are initiated and samples are analyzed by UV-vis spectroscopy, HPLC, and NMR. By measuring absorbance at 255 nm over time, real-time kinetics can be measured by UV. HPLC confirms the expected product, PtCN₄2−, and provides a second method to observe real-time kinetics when ligands interfere at 255 nm. NMR is used to observe reactions using 13C-KCN. The extent of reactions is analyzed by quantifying the product formed and comparing the result to the amount of starting platinum complex utilized. Measuring the extent of reaction over a period of days or weeks gives a measure of “functional stability” for each formulated platinum complex. Stability testing of the aged formulations follow the same methodologies. These will be stored ambient samples or accelerated by storage at 37° C. for 3 days. The samples are expected to maintain >85% capacity to capture cyanide in solution.

### Example 17

### Zebrafish Cyanide Protection

This assay is now well established and used for initial evaluation of cyanide protection of each agent or formulation. The primary metric to determine is EC₁₀₀ for each candidate. The cyanide concentrations used in this screen are 50 μM. A cutoff for the performance of the Pt(II) agents will be set at 4 the cyanide concentration (i.e., 12.5 μM) based upon the optimal conversion to Pt(CN)₄²⁻.

In parallel, the LD₁₀₀ of each candidate is determined in non-cyanide-treated zebrafish. The cutoff LD₁₀₀ is 250 μM or 5× the cyanide dose. Briefly, zebrafish larvae (6 d.p.f.) are loaded in 96-well plates. An estimated 480 larvae are per 96-well plate for each of a vehicle, positive and negative controls, and a 10-point dose-response curve per drug.

Compounds are screened using a 10-point dose-response analysis (0.4-500 μM).

In control animals, KCN is added at a dose of 50 μM which induces 100% death within 1 hour. Following the addition of cyanide, the plates are sealed with adhesive PCR plate foil and incubated at 28° C.

The lowest effective dose to rescue is 100% of larvae; in such case, (EC₁₀₀) will be reported 4 hours post-treatment. For assessment of compound toxicity, larvae are treated for 24 hours with compounds and viability is assessed by observing heart rate and touch response. The dose that causes 100% lethality will be reported (LD₁₀₀).

All new active candidate Pt(II) agents are re-tested as pre-formulated materials with established pH, buffer, and ionic strengths on days 1 and 7. The results of any changes in the EC₁₀₀ are correlated with the stability studies of cyanide reactivity to ensure that the cutoff criterion in FIG. 53 are satisfied.

While using sulfide ligands enhances the rate of cyanide addition, it can also accelerate sulfhydryl addition, other biomatrix components, and even water. This unpredictable speciation is a challenge even with the in vitro analytical methods. Thus, using the in vivo zebrafish model to track the efficacy of cyanide rescue can assess the developability of a formulated active ingredient.

### Example 18

### Nephrotoxic Potential Studies of Pt(II) Agents

Drug-induced nephrotoxicity is an important consideration in developing platinum-based therapeutics, and de-risking novel platinum entities early in the development pipeline is critical. Potential adverse events include glomerular injury (leading to proteinuria and hypoalbuminemia due to improper filtration of large molecules), tubular injury (displaying hypophosphatemia and hypokalemia due to lack of reabsorption in the tubules), crystal nephropathy (characterized by crystalline deposits in the urine), and kidney inflammation (which reduces renal blood flow and glomerular filtration).

Nephrotoxicity is the most common adverse event associated with platinum drugs, especially cisplatin. Cisplatin is known to damage the kidney and cause tubular injury and inflammation. Because of their similarity to cisplatin, assessing this toxicity is very important for developing platinum compounds as cyanide antidotes. Following the 3R principle, well-established in vitro models are utilized to assess the nephrotoxic potential of the compounds before animal studies and a zebrafish model is utilized to act as a bridge between in vitro and in vivo mammalian models. Successful completion of these studies allows for the assessment of renal toxicities posed by the present compounds while minimizing the number of rats used in definitive toxicity studies.

Zebrafish have conserved renal physiology as compared to mammals and are an established model for assessing renal toxicity. Zebrafish larvae as young as four days post-fertilization possess a functional pronephros structure that runs laterally along the length of the larva. Zebrafish (larvae and adults) also exhibit similar cisplatin-induced toxicities including neurotoxicity, ototoxicity and renal damage.

LLC-PK1 cells (derived from porcine proximal tubules) can differentiate the toxicity of cisplatin and oxaliplatin in a trans-well permeability assay. These responses to cisplatin occur at drug concentrations within the range of nephrotoxic blood levels in the rat model. Permeability of the monolayer is assessed using fluorescent dextran. Cisplatin (30 μM) but not oxaliplatin (30 μM) disrupts the barrier in these cells causing fluorescent dextran to leak into the outer chamber. The impact of our candidate Pt(II) agents on barrier integrity is assessed by measuring the fluorescent signal in the outer chamber. Any concentrations disrupting the LLC-PK1 barriers are interpreted in the context of drug levels found in blood in pharmacokinetic evaluations. It is predicted that the toxic doses in rats will mirror concentrations disrupting polarized LLC-PK1 layers, as previously shown with cisplatin.

Briefly, LLC-PK1 cells are grown to confluence on transwell membranes. Prior to the start of the assay barrier integrity is determined by measuring transepithelial electrical resistance with an Epithelial Voltohmmeter (WPI) and subsequently treated with compounds at 3 doses. Cisplatin (30 μM) will be used as positive control and oxalaplatin as a negative control (30 μM). Barrier integrity is assessed using fluorescent dextrans. Fluorescent signal in the media in the outer chamber is measured on a spectrophotometer. In addition, a cell viability assessment is conducted using CellTiter-Glo® Assay.

Secondly, cytotoxicity in this cell line is evaluated. It is expected that concentrations required to kill cells will be far higher than those required to disrupt the barrier. The compounds are ranked from least to most toxic based on these in vitro assays.

The toxicity of platinum compounds has features, such effects on blood flow and activation of the immune system. Zebrafish models faithfully capture these features and as a lower vertebrate organism is aligned with NIH's 3R rule. The zebrafish model is used for assessing nephrotoxicity and evaluating the effect of the present platinum compounds on renal function in vivo.

Previously, an assay was developed to measure glomerular filtration in zebrafish directly. Control compounds (such as cisplatin, carboplatin and oxaliplatin) are used in each assay. The effects of the candidate Pt(II) cyanide antidotes on glomerular filtration are measured using fluorescent dextran. It is expected that compounds known to increase BUN and CREA in the rat will lower glomerular filtration in zebrafish. The ranking, from least to most toxic, in this zebrafish assay will be compared to rankings from LLC-PK1 cell assays.

Zebrafish larvae (5 d.p.f) are exposed to compounds for 4-24 hours. Cisplatin (30 μM) is used as positive control and oxalaplatin as a negative control (30 μM). Subsequently, they are anesthetized and micro-injected with 10-kDa FITC-dextran in the heart. Z-stack images are acquired for each animal at 1, 6, and 24 hours post-injection. To quantitative dextran clearance, maximum intensity projection of image stacks is created and the fluorescent signal in the same region of the dorsal aorta and cardinal vein caudal for each animal will be analyzed in ImageJ to quantify fluorescent intensity over time. Animal numbers: 25 larvae/group×5 groups (vehicle, positive control, and platinum complex at 3 doses)=125 larvae/assay.

Rats are an accepted model for testing for renal toxicity. For each study, animals (6 males/6 females Sprague-Dawley) are treated with 1× and 5× the EC₁₀₀ (allometrically scaled) observed in the mouse cyanide inhalation model via IP administration. Blood chemistry is assessed on day 1 and day 7 after treatment. The increases in both BUN and CREA are interpreted as a lowering of glomerular filtration. In isolation, each of these biomarkers can be nonspecific. A lack of hypoalbuminemia, hypophosphatemia, or hypokalemia supports glomerular damage and loss of tubular resorption, which can result from tubular cell death, may not occur. Similar to cisplatin, these observations would be in agreement with potential inflammatory effects. Inflammation, caused by direct and secondary effects of platinum agents, can reduce glomerular filtration (and thus higher serum BUN and creatinine) independent of hypoalbuminemia, hypophosphatemia, and hypokalemia.

### Example 19

### Efficacy Studies in Mouse Model of Cyanide Inhalation

Lead Pt(II) candidates and/or their new formulations advance to murine studies based on the criteria outline in FIG. 53.

A stringent model was developed to simulate a real-life cyanide exposure scenario. Mice are exposed to HCN gas, injected intramuscularly with antidote(s), and then re-exposed to HCN. This model assumes about 15 minutes will be required for emergency medical personnel to arrive at a disaster scene and another 25 minutes to treat and evacuate the cyanide-exposed persons from the contaminated area. A custom-made sealed chamber is used in a chemical fume hood to minimize the risk of exposing laboratory personnel to cyanide, but allows for visual monitoring of the animals. The chamber is pre-heated to 30° C., and a mouse is placed into the chamber. Liquid isoflurane is injected to achieve a concentration of 2%.

IACUC requires the chamber to be warm for the mice; the elevated temperature additionally helps vaporize the isoflurane and cyanide gas. The mice become anesthetized within 2 minutes, at which time KCN is injected into a beaker containing HCl and a magnetic stir bar. The apparatus sits on top of a magnetic stir plate, so the KCN is mixed quickly with the acid to generate HCN. A circulating fan within the chamber assures rapid equilibration so that the HCN concentration reaches a steady state within 5 minutes, remaining constant for up to 1 hour. Accurate control of the gas concentration over a wide range of concentrations can be achieved.

These studies have two animal groups: vehicle control and Pt(II candidate treated. The outcome is survival and the criteria for down-selection is ≥90% survival in an assay with 100% lethality.

Animal Numbers: Sample size was determined using a Chi-square test, setting alpha at 0.05 and power at 0.9. 100% lethality in untreated mice is expected. Aiming for at least 90% survival in treated animals, a sample size of 11 was calculated for each group. A corresponding number of control saline-treated mice are used for each group, yielding 24 mice [2 groups×6 animals/group×2 sexes]. Comparisons are between treated and untreated animals of the same group and gender. Survival curves are generated and analyzed using a log-rank test. Clinical evaluation of the mice is evaluated dichotomously as either normal or abnormal and using a standard t-test.

### Example 20

### Evaluation of the Metabolite Glyoxylate with Pt(II)-Based Scavenger Agents

The metabolite glyoxylate is evaluated with platinum (II)-based scavenger agents. Glyoxylate or other agents that mitigate the effects of cyanide do not eliminate cyanide from the body. At the same time, using metals as scavengers for cyanide carries liabilities of toxicities (i.e., renal for Pt(II)). Therefore, a chelating agent is combined with a metabolic modulator to achieve unparalleled countermeasure efficacy with enhanced therapeutic windows to ≥10 (FIG. 54).

The combination of a Pt(II) cyanide scavenger and glyoxylate rescues cyanide-poisoned zebrafish and mice (FIG. 54). In zebrafish and mice, the combination of glyoxylate with a Pt complex, at doses in which each single agent is ineffective, protected animals from lethal cyanide exposure. At the listed doses of PCP or glyoxylate alone, 0% survival was observed.

In zebrafish, glyoxylate has an EC₁₀₀=32 μM and doses≤15 μM were ineffective, while PCP has an EC₁₀₀=62 μM and doses≤32 μM were ineffective. In combination studies, 15 μM of PCP plus 8 μM glyoxylate rescued 100% of zebrafish. Concordant findings were observed in mice (FIG. 55). Glyoxylate (60 mg/kg; n=6) or PCP (31.5 mg/kg; n=6) administered alone resulted in 16% survival, while the combination of the two agents resulted in 66% (n=6).

A separate study evaluated the chelation affinity of glyoxylate for cyanide using ¹³CNMR to monitor cyanohydrin formation (FIG. 56). In addition, several keto metabolites were included for comparisons. The pyruvate equilibrium constant with cyanide was established as Kₐ=11 mM-1 or K_{D}=90 M. A competition experiment between pyruvate and glyoxylate (or any other keto metabolite) was used to directly evaluate the dissociation constant for cyanide (FIG. 56). These results set the stage for selective Pt(II) chelation with cyanide (>104 higher affinity) when in the presence of glyoxylate ensuring the metabolite will be available for metabolic rescue.

These studies rely initially on the published Pt(II) complex bearing two methionine ligands (Met₂Pt) being in hand. Preliminary data indicate that Met₂Pt, when dosed at 5×EC₁₀₀ in the rodent model exhibits, elevated CREA and BUN in rats (not shown). A reduction in the Pt(II) effective dose by a factor of 2× widened the therapeutic window to 5.

Preliminary NMR data (H, ¹³C, ¹⁹⁵Pt) data indicate that a sample of 3:1 glyoxylate/Met2Pt remained unchanged over 3 days (not shown). These preliminary data show feasibility of co-formulation of glyoxylate with a reactive Pt(II) complex.

Combinational agent testing in the zebrafish cyanide assay is conducted initially in the presence of a sub-effective dose of 8 μM glyoxylate. This level of glyoxylate represents ⅕ of the total cyanide. A Pt(II) complex, in theory, must present at at least 12.5 μM to consume all of the cyanide under these conditions. However, the minimal amount of cyanide scavenging required to sustain the zebrafish is not known at this time. Therefore, a testing approach will strive to reduce the overall burden of metal administered by finding an optimal ratio with glyoxylate.

Platinum complexes are tested at doses of 0.4-12.5 μM. Platinum compounds that exhibit increases in potency as a change in EC₁₀₀≥2 and/or a 2-fold improvement in nicotinamide adenine dinucleotide (NADH)/NAD when combined with glyoxylate advance. Fixation of cyanide on cytochrome c oxidase prevents its reoxidation by oxygen and thereby causes a backup of electrons on the electron transport chain. Complex I is subsequently stuck in a reduced state and therefore cannot reduce NADH regenerate the NAD+ that is required for the tricarboxylic acid (TCA) cycle. As NADH/NAD+ ratio increases, negative feedback inhibition on the TCA cycle ensues, which forces the cell to shift from aerobic to anaerobic metabolism.

The NADH/NAD+ ratio is measured in lysates from cyanide-treated zebrafish larvae (6 d.p.f.) using an enzymatic assay (Abcam ab65348) and compared to cyanide-treated animals with lead candidates and vehicle-treated (no cyanide). In addition, the lactate:pyruvate ratio is also assessed in zebrafish lysates as it is in near equilibrium with the NADH:NAD+ ratio as a second confirmation.

Finally, the cystine/cysteine ratio is measured in zebrafish lysates as a surrogate marker of extracellular redox stress that improves in cyanide-poisoned swine treated with glyoxylate. These 4 metabolites are assessed using targeted mass spectrometry methods in positive ion mode on a Sciex 4000 QTRAP triple-quadrupole mass spectrometer (cystine, cysteine), and negative ion mode on an Agilent 6490 QQQ triple-quadrupole mass spectrometer (pyruvate, lactate). Combinations that exhibit a 2-fold improvement in NADH:NAD+ and pyruvate/lactate, in addition to restoring cystine/cysteine ratio to baseline, are advanced to mouse studies.

For the mouse studies, there are three groups: vehicle control, platinum complex (EC₅₀), and combination of agents. For each group, a corresponding number of control saline-treated mice are required, yielding 36 mice [3 groups×6 animals/group×two sexes]. The target concentration for the glyoxylate is an ineffective dose of 40 mg/kg (⅓ EC₁₀₀) and the Pt(II) complex is <EC₅₀. The current dose of glyoxylate used in the large animal swine model corresponds to 3 mole equivalents relative to the cyanide dose.

These doses are adjusted as the information from the zebrafish efficacy and the nephrotoxicity studies emerge. For instance, if the glyoxylate:Pt(II) ratio in a combination is refined in the zebrafish kidney function assay to reduce or eliminate signs renal dysfunction, then the efficacy of that ratio in cyanide toxicity model is retested before advancing to the mouse model.

### Example 21

### Efficacy & Safety Studies of Pt(II)-Based Agents Alone and in Combination with the Metabolite Glyoxylate in Porcine Models for Cyanide Intoxication

The FDA requires efficacy studies in at least two mammalian animal models to obtain FDA approval via the animal rule. The swine model enables the collection of unique information not captured in the mouse or zebrafish models, including continuous hemodynamic data, clinical laboratory values, and serial blood sampling for metabolite profiling and PK studies. The EC₈₀ of the identified lead candidate(s) is determined and the EC₈₀ in fixed ratio combinations with glyoxylate. This large animal model provides a robust test for translatable cyanide countermeasures delivered as a single intramuscular injection.

Acute cyanide poisoning leads to hypoventilation and apnea, and patients presenting with respiratory failure have an increased mortality risk. Therefore, the primary models are spontaneously breathing animals with a well-established, non-ventilated, intravenous infusion, swine model. Since the pKₐ of HCN is 9.2, cyanide exists almost exclusively as HCN at physiological pH, infusing a cyanide salt generates HCN, the form of cyanide absorbed from the lungs or stomach. A cyanide infusion model yields the same end product as an inhalation or ingestion model, but has the advantage of knowing the exact amount of cyanide the animal receives. Furthermore, the swine model mimics the physiological effects of cyanide poisoning observed in humans (apnea and cardiovascular collapse). Additionally, swine also make a good model for evaluating pharmacokinetics and efficacy due to their large size and the fact their circulatory system is similar to humans.

Assessment of the metabolic effects of glyoxylate as compared to a cyanide scavenger in swine was performed. Preliminary data established that glyoxylate exhibits evidence for redox metabolism effects that reverses cyanide exposure's impact in the porcine model (83% survival at 10 mg/kg IM, n=6; FIG. 57).

Next, the circulating ratio of lactate:pyruvate was measured as it is in near equilibrium with intracellular NADH:NAD+ ratio. At the end of the cyanide infusion, plasma lactate:pyruvate decreased by 36% as compared to baseline levels in swine (FIG. 58A, gray box; p=0.01).

The elevated lactate levels suggests a defect in mitochondrial oxidative phosphorylation during cyanide exposure. Following administration of glyoxylate, the lactate:pyruvate ratio rapidly increased from 0.66±0.14 to 1.99±0.39 at 7.5 minutes post-injection (FIG. 58A; p=0.006), indicating rapid production of lactate or consumption of pyruvate in response to glyoxylate. Subsequently, the lactate:pyruvate ratio declined to slightly below baseline by 45 minutes post-injection (0.82±0.29). To assess the specificity of these findings, the lactate:pyruvate ratios of glyoxylate were compared to that of a known cyanide-chelating agent (HCP formulated with DMSO).

It was demonstrated that HCP (20 mg/kg IM) restores lactate:pyruvate to baseline levels at 7.5 minutes post-treatment, however glyoxylate exerted effects on lactate:pyruvate were distinct from HCP (FIG. 58A). The burst in lactate:pyruvate ratio observed in glyoxylate-treated swine, between t=0 to t=15 minutes, did not occur in HCP-treated animals (1.99±0.39 versus 1.15±0.28, p=0.0418). The effect of glyoxylate on both pyruvate consumption and lactate production, in conjunction with the lactate:pyruvate ratios, suggests that glyoxylate administration in cyanide-poisoned animals alters metabolic pathways in a manner that is distinct from a known cyanide scavenger. These findings also implicate that glyoxylate administration transiently shifts the balance of intracellular NADH/NAD+.

The plasma ratio of Cys:CySS is an established biomarker for extracellular redox balance. During cyanide infusion, the extracellular redox environment shifted to an oxidized state as evidenced by decreased plasma Cys:CySS. At the end of the cyanide infusion, Cys:CySS decreased by 27% (p=0.04) as compared to baseline (FIG. 58B). Following IM injection of glyoxylate, the ratio of Cys:CySS increased from 0.73±0.27 to a peak ratio of 2.11±0.78 at 60 minutes post-injection (FIG. 58B; p=0.01).

These results indicate that the extracellular redox environment shifted to a reduced state in glyoxylate-treated animals. In contrast to glyoxylate, HCP did not improve the cyanide-induced shift to an oxidized state (FIG. 58B). Following HCP-administration, Cys:CySS continued to remain below baseline until the end of the study (0.56±0.09 versus 1.00). The overall results suggest that cyanide chelation alone is not sufficient to ameliorate cyanide-induced oxidative stress in the extracellular compartment.

These data demonstrate beneficial biological effects, independent of scavenging, that are unique to glyoxylate as compared to a cyanide chelator. However, as a single agent, glyoxylate efficacy may be less than optimal. The current IM dosing levels required for glyoxylate are larger than desired volumes for an autoinjector. In addition, the molar equivalence of glyoxylate required to reach EC₈₀ in the swine model is 3× greater than the total cyanide infused. At this time, the risks of a large amount of oxalate formation from the glyoxylate is not clear. However, large amounts of crystalline oxalate is observed in the urine of the swine. Accordingly, it would beneficial to provide a combination therapy of glyoxylate and the platinum complex candidates hereof.

The preliminary results indicate that Pt-sulfide bidentate ligand complexes exhibit utility as potential cyanide countermeasures in the same animal models. The prior results with DMSO-formulated HCP also supports that potential efficacy in the large animal swine model. In order to further leverage these observations, additional lead Pt(II) complex candidates (Example 16) is advanced through the evaluation process to testing in the swine model. The workflow and target performance criteria are outlined in FIG. 59.

The target dose of Pt is justified as being a quarter of the total dose of cyanide in the swine model. This represents an optimal scenario for capture of all infused cyanide as Pt(CN)₄₂. The product concept for the field-ready countermeasure is the use of an autoinjector with ready filled formulation. Current autoinjectors are on the order of 3 mL, but it is anticipated the availability of a newer technology for volumes up to 5 mL. The time to animal recovery of breathing is arrived at from empirical testing with the swine model with other cyanide scavenger agents. In the same way, the peak blood level concentrations of the active scavengers are anticipated as a critical criterion for reproducible performance (FIG. 60).

Candidate agents emerging from Examples 16-19 are heavily qualified in the mouse model before being tested in the swine. Scale up synthesis and full batch characterizations of the candidate formulations are completed 1 month before a planned animal study as described in Example 20. Attempts to minimize ionic strength and pH effects of the formulations are ongoing.

The dose of a lead platinum complex that rescues 80% of animals in a model that has >80% lethality in control treated animals is established by starting with the effective dose determined in the mouse studies and scaling to pig using allometric dose scaling. The platinum complex is administered intramuscularly in two animals per dose, up to three doses (6 pigs) to determine the optimal dose. Once the optimal dose is determined, a full efficacy study using male and female swine (20 pigs) is performed. Swine are exposed to cyanide via intravenous infusion of potassium cyanide until apnea (cessation of breathing) occurs, at five minutes post apnea treatment with either platinum complex or control occurs, and the cyanide infusion is stopped. Following treatment, animals are monitored for 90 minutes. Outcomes include survival, return of respiration, normalization of cardiovascular parameters, and normalization of blood gas (pH, arterial blood oxygenation) and lactate.

A dose of the lead platinum complex is established in combination with 3.5 mg/kg glyoxylate (⅓ EC₁₀₀) that rescues 80% of animals in a model that has >80% lethality in control treated animals. Administering the effective dose of the platinum complex determined in the previously described study in 2 pigs and adjust the dose for up to three doses (6 pigs) to determine the EC₈₀. When the optimal dose of the combination therapy is determined, a full efficacy study is performed using male and female swine (20 pigs). Swine are exposed to cyanide via intravenous infusion of potassium cyanide until apnea (cessation of breathing) occurs, at five minutes post apnea, treatment with either platinum complex or control is administered, and the cyanide infusion is stopped. Following treatment, the animals are monitored for 90 minutes. Outcomes include survival, return of respiration, normalization of cardiovascular parameters, and normalization of blood gas (pH, arterial blood oxygenation) and lactate.

Yorkshire cross swine, 50 kg in weight, are used for these studies. Following the study, they are anesthetized with intramuscular ketamine (10 mg/kg), intubated, and sedation maintained with 1-2% inhaled isoflurane while breathing room air spontaneously. They receive both full cardiovascular and respiratory monitoring. Potassium cyanide is infused intravenously at 0.17 mg/kg until apnea (<6 breaths per minute). At predefined times after apnea, the cyanide infusion is stopped and saline or the lead candidate is administered intramuscularly. Arterial blood gases, blood chemistries, and blood lactate are monitored every 20 minutes. Serial blood samples are collected for metabolite profiling and PK studies. In surviving animals, muscle biopsies are obtained to evaluate muscle integrity at the injection site.

Pharmacokinetics and toxicity of the compounds hereof are assessed in non-cyanide exposed swine since cyanide exposure alters cardiovascular function resulting in altered hemodynamics, and likely drug distribution. The pharmacokinetic of the platinum complexes are independently assessed using the dose used in the efficacy study described above, the dose administered via intramuscular injection. The pharmacokinetics of the combination therapy is then assessed, platinum complex and glyoxylate administered intramuscularly at the dose used in the previously described efficacy study. Drug concentration and blood chemistry is measured (including hepatic and renal function) at baseline, and at 5, 15, and 30 minutes, at 1, 2, 4, 8 hours, and at 1, 3, and 7-days post injection. For each study, baseline blood samples are obtained. We plan to have five animals in each group, two males and three females to yield a total of ten pigs for these studies. Plasma derived from the blood samples is divided for two arms of bioanalyses (FIG. 59); the first processed for platinum metal analysis using ICP-MS, and the second arm of bioanalysis being metabolic profiling using an established HPLC-MS platform.

A log-rank test is used to compare survival in control and treatment arms; to account for potential effects of sex, survival differences is confirmed using a sex-stratified log-rank test. Sample sizes of 10 animals in control and treatment arms yield 90% power (alpha 0.05) to distinguish between 80% and 20% survival for treatment and control arms, respectively. Metabolite data is Bonferroni corrected. For the pharmacokinetic studies, the mean values of the blood concentration curve are plotted for each group. For PK analyses, the data is analyzed initially using non-compartmental approaches, which provides estimates of the clearance, half-life, volume of distribution, mean residence time, maximal drug concentration, and time of maximum concentration. Following this analysis, the data is evaluated by compartmental modeling (one- and multi-compartment models), and the model that best fits the data is selected.

1. A compound having a structure of Formula (I): 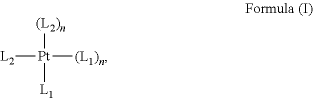
or a pharmaceutically acceptable salt, N-oxide, solvate, tautomer, or stereoisomer thereof, wherein:
Pt is platinum(II);
each L₁ and L₂ is a ligand, wherein each L₁ forms a bidentate ligand and each L₂ forms a bidentate ligand, each bidentate ligand comprising an N, an S, or both an N and an S coordinated to the platinum(II), wherein at least one of the ligands is a leaving group and at least two of the ligands that are directly bonded to the platinum(II) each independently comprise a thioether comprising an alkyl, a carboxyamide, an amine, an amino sulfide, a carboxylate, a carboxyester, a carbonyl, or a combination of any of the foregoing; and
each n is independently −1-5.

2. A compound having a structure of Formula (II): 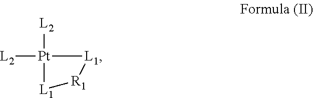
or a pharmaceutically acceptable salt, N-oxide, solvate, tautomer, or stereoisomer thereof, wherein:
Pt is platinum(II);
each L₁ and L₂ is a ligand, wherein each L₁ forms a bidentate ligand and each L₂ forms a bidentate ligand, each a bidentate ligand comprising an N, an S, or both an N and an S coordinated to the platinum(II), wherein at least one of the ligands is a leaving group and at least two of the ligands each independently comprise a thioether comprising an alkyl, a carboxyamide, a carboxyester, an amine, an amino sulfide, a carboxylate, a carbonyl, or a combination of any of the foregoing;
R₁ is selected from a group consisting of C₁₋₃ alkyl, C₁₋₃ haloalkyl, C₂₋₄ alkenyl, C₂₋₄ alkynyl, and C₆₋₁₀ aryl, or absent; and
each n is independently −1-5.

3. A compound having a structure of Formula (III): 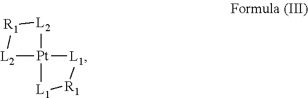
or a pharmaceutically acceptable salt, N-oxide, solvate, tautomer, or stereoisomer thereof, wherein:
Pt is platinum(II);
each L₁ and L₂ is a ligand, wherein each L₁ forms a bidentate ligand and each L₂ forms a bidentate ligand, each a bidentate ligand comprising an N, an S, or both an N and an S coordinated to the platinum(II), wherein at least one of the ligands is a leaving group and at least two of the ligands that are directly bonded to the platinum(II) comprise a thioether comprising an alkyl, a carboxyamide, an amine, an amino sulfide, a carboxylate, a carbonyl, or a combination of any of the foregoing;
each R₁ is selected from a group consisting of C₁₋₃ alkyl, C₁₋₃ haloalkyl, C₂₋₄ alkenyl, C₂₋₄ alkynyl, and C₆₋₁₀ aryl, or absent; and
each n is independently −1-5.

4. The compound of any one of claims 1-3, wherein each L₁ and L₂ is a leaving group.

5. The compound of claim 2 or 3, wherein each R₁ is absent.

6. The compound of claim 1, comprising a cis configuration.

7. The compound of claim 1 comprising a trans configuration.

8. The compound of any one of claims 1-3, 6 and 7, wherein a first ligand of a bidentate ligand comprises a sulfide and a second ligand of the bidentate ligand comprises an amide.

9. The compound of any one of claims 1-3, 6 and 7, wherein at least one thioester ligand comprises an amino sulfide.

10. The compound of any one of claims 1-3, 6 and 7, wherein the thioester ligands each comprise an amino sulfide.

11. The compound of any one of claims 1-3, 6 and 7, wherein the bidentate ligands independently comprise 5- or 6-membered bidentate ligands.

12. The compound of any one of claims 1-3, 6 and 7, wherein the compound has the following structure: 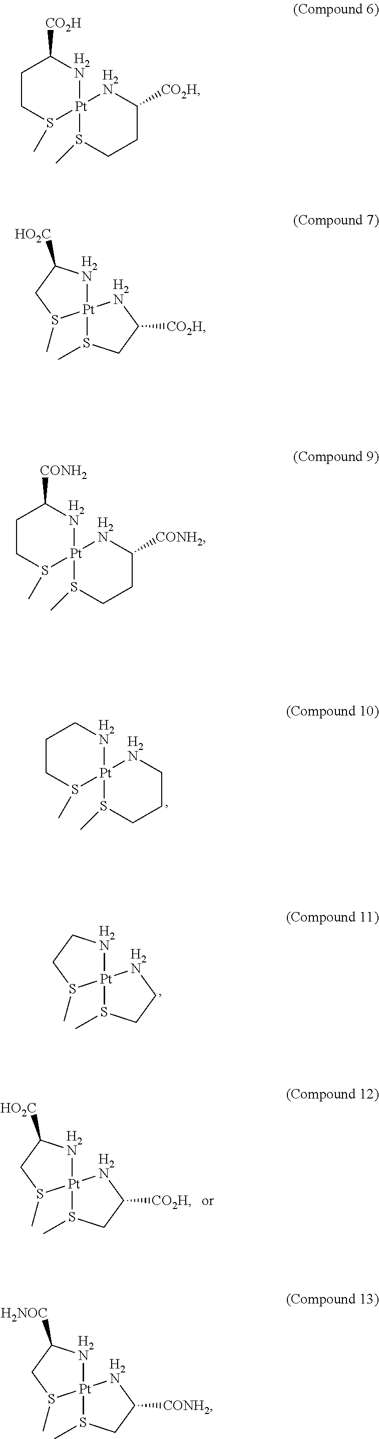
or is a pharmaceutically acceptable salt, N-oxide, solvate, tautomer, or stereoisomer of any of the foregoing structures.

13. The compound of claim 1 comprising the following structure or a pharmaceutically acceptable salt, N-oxide, solvate, tautomer, or stereoisomer thereof.

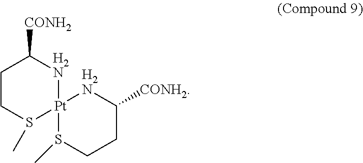

14. The compound of claim 1 comprising the following structure or a pharmaceutically acceptable salt, N-oxide, solvate, tautomer, or stereoisomer thereof

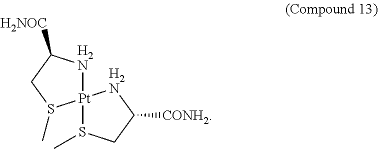

15. The compound of any one of claims 1-3, wherein at least one of the ligands comprises a methyl thioether group.

16. The compound of any one of claims 1-3, wherein at least one of thioether ligands comprises methionine or S-methylcysteine optionally comprising one or more amidated carboxylates.

17. The compound of any one of claims 1-3, 5 and 6, wherein the bidentate ligands independently comprise 5- or 6-membered bidentate ligands, and at least one of the bidentate ligands comprises a carboxylate or carboxamide substituent.

18. The compound of claim 2 or 3, wherein R₁ is a C₁₋₃ alkyl.

19. The compound of any one of claims 1-3 comprising HCP-AKN, cisplatin-AKN, (SallylCys)₂Pt, (SMePenicillamine)₂P, (cilastatin)₂Pt, or (bridged-Met₂)Pt, MetPt(taurine)₂.

20. A pharmaceutical composition comprising:
a compound of any one of claims 1-19 or a pharmaceutically acceptable salt, N-oxide, solvate, tautomer, or stereoisomer of a compound of any one of claims 1-19; and
a pharmaceutically acceptable carrier and/or diluent.

21. The pharmaceutical composition of claim 20, further comprising a pharmaceutically acceptable excipient.

22. The pharmaceutical composition of claim 20, wherein the composition is suitable for intramuscular injection.

23. The use of a compound of any one of claims 1-19, a pharmaceutical salt, N-oxide, solvate, tautomer, or stereoisomer of a compound of any one of claims 1-19, or a pharmaceutical composition of any one of claims 20-22 in the manufacture of a medicament for the treatment of a disease or condition in a subject.

24. The use of claim 23, wherein the disease or condition is cyanide poisoning or cyanide exposure.

25. The use of claim 23, wherein the medicament is formulated for intramuscular administration.

26. The use of claim 23, wherein the medicament is formulated in a single-bolus dosage.

27. The use of claim 23, wherein the medicament is formulated at about or above a pH of 5.

28. The use of claim 23, wherein the medicament is stored at about or below a pH of 5.

29. A method of treating cyanide poisoning or cyanide exposure in a subject comprising administering to the subject a first therapy comprising a therapeutically effective amount of:
a compound of any one of claims 1-19;
a pharmaceutically acceptable salt, N-oxide, solvate, tautomer, or stereoisomer of a compound of any one of claims 1-19; or
a pharmaceutical composition of any one of claims 20-22.

30. The method of claim 30, wherein administering comprises intramuscular injection.

31. The method of claim 30 or 31, wherein administering the therapeutically effective amount of the first therapy comprises administering a single dose.

32. The method of claim 30, further comprising administering to the subject a second therapy, the second therapy comprising administering to the subject:
a therapeutically effective amount of one or more of glyoxylate, hydroxocobalamin, methemoglobin, riboflavin, methotrexate, 4-dimethylaminophenol (4-DMAP), dicobalt edetate, glucose, activated charcoal, and cobalt disodium ethylenediaminetetraacetic acid (EDTA);
an isotonic fluid intravenously; and/or
oxygen therapy.

33. The method of claim 32, wherein the first and second therapies are administered sequentially and/or alternatively relative to each other.

34. The method of claim 32, wherein the first and second therapies are administered concurrently.

35. The method of claim 32, wherein the second therapy is glyoxylate and the therapeutically effective amount of the first therapy is about 3.0-5.5 mg/kg (by weight of the subject).

36. The method of claim 35, wherein the therapeutically effective amount of the first therapy is about 3.5 mg/kg (by weight of the subject).

37. The method of claim 30, wherein the therapeutically effective amount is about 3.0-5.5 mg/kg (by weight of the subject).

38. The method of claim 30, wherein at least one ligand of the compound, pharmaceutically acceptable salt, N-oxide, solvate, tautomer, or stereoisomer of the compound, or pharmaceutical composition comprises an amino acid ligand substituted with one or more carboxamides.

39. The method of claim 30, wherein administering comprises intramuscular injection of the compound, pharmaceutically acceptable salt, N-oxide, solvate, tautomer, or stereoisomer of the compound, or pharmaceutical composition into the subject and wherein the compound or pharmaceutical composition reaches maximum concentration in the subject at about or between 7-9 minutes post administration (such as at or between 7 minutes to about 9 minutes, at or between about 7 minutes to 9 minutes, or at or between 7 minutes to 9 minutes).

40. The method of claim 38, wherein, after administered intramuscularly to the subject, the compound, pharmaceutically acceptable salt, N-oxide, solvate, tautomer, or stereoisomer of the compound, or pharmaceutical composition undergoes pH-induced isomerization resulting in a reduced cyanide scavenging rate as compared to a cyanide scavenging rate of the compound, pharmaceutically acceptable salt, N-oxide, solvate, tautomer, or stereoisomer of the compound, or pharmaceutical composition within 1 hour of administration to the subject.

41. A combination therapy for treating cyanide poisoning or cyanide exposure in a subject comprising administering to the subject:
a therapeutically effective amount of cyanide chelating agent; and
a therapeutically effective amount of an agent for ameliorating cyanide-induced oxidative stress in the subject.

42. The combination therapy of claim 41, wherein the cyanide chelating agent comprises a platinum(II) thioether comprising bidentate ligands.

43. The combination therapy of claim 41 or 42, wherein the cyanide chelating agent comprises:
a compound of any one of claims 1-19;
a pharmaceutically acceptable salt, N-oxide, solvate, tautomer, or stereoisomer of a compound of any one of claims 1-19;
a compound or pharmaceutically acceptable salt, N-oxide, solvate, tautomer, or stereoisomer having the following structure: 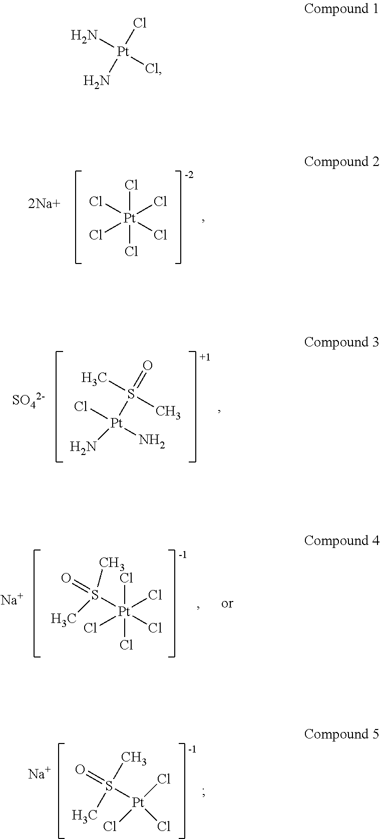
a pharmaceutical composition of any one of claims 20-22.

44. The combination therapy of claim 42 or 43, wherein the agent for ameliorating cyanide-induced oxidative stress in the subject is glyoxylate or an analog or functional fragment thereof.

45. The combination therapy of claim 42 or 43, wherein the agent for ameliorating cyanide-induced oxidative stress in the subject comprises a ketone or aldehyde compound selected from the group consisting of glyoxylate, glyceraldehyde, acetaldehyde, dihydroxyacetone, pyruvate, methylglyoxal, and alpha-ketoglutarate.

46. A kit for treating cyanide poisoning or cyanide exposure comprising:
a drug injection device comprising one or more fluid chambers prefilled with a first formulation comprising:
a compound of any one of claims 1-19,
a pharmaceutically acceptable salt, N-oxide, solvate, tautomer, or stereoisomer of a compound of any one of claims 1-19,
a compound or pharmaceutically acceptable salt, N-oxide, solvate, tautomer, or stereoisomer having the following structure: 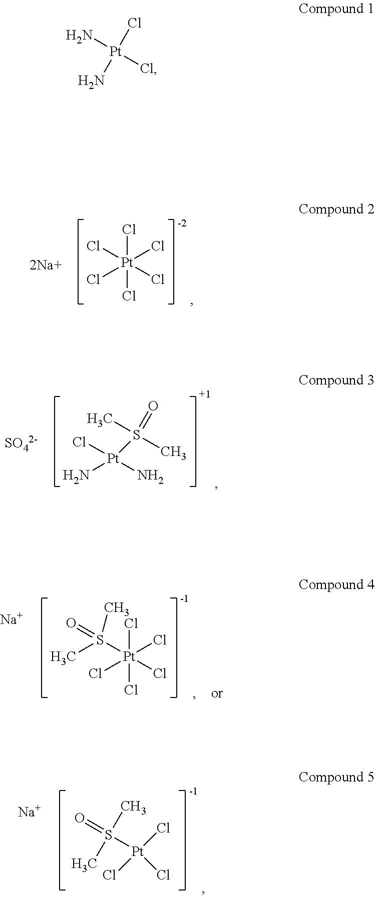
and
a pharmaceutically acceptable carrier and/or excipient.

47. The kit of claim 46, wherein the first formulation comprises a targeted effective dose of the compound or pharmaceutically acceptable salt, N-oxide, solvate, tautomer, or stereoisomer for intramuscular injection.

48. The kit of claim 46, wherein the prefilled fluid chamber is a syringe or a cartridge.

49. The kit of any one of claims 46-48, wherein the formulation has a pH value of 5 or less.

50. The kit of any one of claims 46-48, wherein the drug injection device is an autoinjector or a hand-held injector.

51. The kit of any one of claims 46-50, further comprising one or more fluid chambers prefilled with a second formulation comprising a therapeutically effective amount of an agent for ameliorating cyanide-induced oxidative stress in the subject and a pharmaceutically acceptable carrier and/or excipient.

52. The kit of claim 51, wherein the agent for ameliorating cyanide-induced oxidative stress in the subject is glyoxylate or an analog or functional fragment thereof.
